SEQUENCE LISTING (204 sequences)

SEQ ID NO: 1 - protein, 124 aa, synthetic construct
QITLKESGGGLVQPGGSLRLSCAASGFTFYSYVMTWVRQAPGKGLEWVSTISDDGRSTYYAESVEGRFTISRDNFKNAVHLQMSSLRAEDTAVYYCVKGLAAVGQGNYFGMDVWGPGTTVTVSS

SEQ ID NO: 2 - protein, 124 aa, synthetic construct
QITLKESGGGVVLPGRSLRLSCAASGFTFNNYAMYWVRQAPGKGLEYVAFISYDGMKTDYADAVQGRFSISRDNSRETLWLQMNSLRADDTAVYFCARDYLLIPRTASNAFDLWGQGTTVTVSS

SEQ ID NO: 3 - protein, 124 aa, synthetic construct
QVQLVQSGGGVVQPGRSLRLSCAASGFTFSNYAMYWVRQAPGKGLEYMAFISYDGTKKDYADSVKGRFSISRDNSRETLWLQMNSLRAEDTAVYFCARDYLLIPRTASNAFDLWGQGTTVTVSS

SEQ ID NO: 4 - protein, 127 aa, synthetic construct
EVQLVESGAELKKPGESLKISCKGSGYSFSSHWIGWVRQMPGKGLEWMGIIHPGDSDTRYSPSFQGQVTISVDKSITTAYLQWSSLKASDTAIYYCAIQGYCRSGRCPEGGDWFDPWGQGTLVTVSS

SEQ ID NO: 5 - protein, 124 aa, synthetic construct
EVQLVESGGGVVQPGRSLRLSCAASGFTFSPYAMHWVRQAPGKGLQWVAFISYDGTKNDYADSVKGRFTISRDNSKNMVFLQMNSLRPEDTALYYCARDILLIPRTTSYAFDIWGQGTTVTVSS

SEQ ID NO: 6 - protein, 121 aa, synthetic construct
QVQLVQSGGGLVQPGRSLRLSCVGSGFSFDDHGMHWVRQVPGKGLQWVSGINWSSVLIGYVESVKGRFTISRDNAKNSLYLQMNSLKPEDTALYFCARGPYSTSQYFFDLWGQGTLVTVSS

SEQ ID NO: 7 - protein, 124 aa, synthetic construct
QVQLVQSGGGVVQPGRSLRLSCAASGFTFSPYAMHWVRQAPGKGLQWVAFISYDGTKNDYADSVKGRFTISRDNSKNMVFLQMNSLRPEDTALYYCARDILLIPRTTSYAFDIWGQGTTVTVSS

SEQ ID NO: 8 - protein, 126 aa, synthetic construct
EVQLVESGPEVKKPGASVKVSCKTSGYTFSTYGINWVRQAPGHGLESLGWISAADGNINYPPKFRGRVTMTRDTATSTVYMELRSLKPDDTAVYYCARDRATFGGPLAAGDALDMWGQGIMVTVSS

SEQ ID NO: 9 - protein, 124 aa, synthetic construct; identical to SEQ ID NO: 3

SEQ ID NO: 10 - protein, 124 aa, synthetic construct
QVTLKESGGGVVQPGRSLRLSCAASGFTFSNYAMYWVRQAPGKGLEYLAFISYDGTKKDYADSVKGRFSISRDNSKETLWLQMSSLRPEDTAVYFCARDYLLIPRTASNAFDIWGQGTTVTVSS

SEQ ID NO: 11 - protein, 123 aa, synthetic construct
QVQLVESGGGVVQPGRSLRLSCVASIFIFENYGMHWVRQAPGKGLEWVAVISHDGRDQYYADSVKGRFSISRDNSKSTVYLQMNSLRPEDTAVYYCAKGVYGGHSIWDLFGDWGQGTLVTVSS

SEQ ID NO: 12 - protein, 121 aa, synthetic construct
QVQLVQSGGGLVKPGGSLRLSCAASGFTFSDYYMTWIRQAPGKGLEWVAYISSGGSSTYDADSVKGRLTISRDNAKNSLYLQMNSLRAEDTAVHYCARVDMGPVSSPIDYWGQGTLVTVSS

SEQ ID NO: 13 - protein, 122 aa, synthetic construct
EVQLVESGGGLVKPGGSLRLSCAASGFTFGDHYMSWVRQAPGKGLEWLAYISTSATYRGYADSVKGRFTISRDNAKNSLYLQMDSLRAEDTAVYFCARDQDSSGYYDAFDIWGQGTTVTVSS

SEQ ID NO: 14 - protein, 121 aa, synthetic construct
QVQLVQSGAEVKKPGASVQVSCKATGYIFTNYYMHWVRLAPGQGLEWMGKINLNGGSTAYAQKFQGRVTMTRDTPTSTVNMELSSLTSEDTAIYYCARGSTSSSKYGLDVWGQGTTVTVSS

SEQ ID NO: 15 - protein, 120 aa, synthetic construct
QVQLVQSGTEVKKPGESLKISCDVSGYTFTTYWIGWVRQKPGKGLEWMGIIYPGDSDIRYSPSFQGQVSISADKSISTAYLQWSSLKASDTAMYYCARRDGSGSGHFDYWGQGTLVTVSS

SEQ ID NO: 16 - protein, 125 aa, synthetic construct
QVQLVQSGGGLVQPGGSLRLSCSAPGFSFSWYWMTWVRQAPGKGLEWVANIKQDGSEKHYVDSVEGRFTISRDNANNSLYLQMNSLRAEDTAVYYCARGVRTVYCGGACYPLGDWGQGTLVTVSS

SEQ ID NO: 17 - protein, 117 aa, synthetic construct
QVQLQQSGGGVVQPGRSLRLSCVGSGFTLRTYGMFWVRQAPGKGLEWLAFISYDGGIKYYADSVKGRFTISRDNSQNTLYLAINSLRTDDTGLYFCARDISGTYNAWGQGPRSPSPQ

SEQ ID NO: 18 - protein, 124 aa, synthetic construct
QVQLVQSGGGVVQPGRSLRLSCAASGFTFSNYAMYWVRQAPGKGLEYLAFISYDGTNKDYADSVKGRFSISRDNSKETLWLQMSSLRPEDTAVYFCARDYLLIPRTASNAFDIWGQGTTVTVSS

SEQ ID NO: 19 - protein, 120 aa, synthetic construct
QVQLVQSGGGLVKPGGSLRLSCAASGFSFINAWMNWVRQAPGKGLEWVGRIKSKTDGGTIEHAAPVRGRFTISRDDSKNTLYLQMTSLKIEDTAIYYCTTGSRGWWFDPWGQGTLVTVSS

SEQ ID NO: 20 - protein, 126 aa, synthetic construct
QVQLVQSGAEVKKPGASVKVSCKASGYTFTSYYMHWVRQAPGQGLEWMGIINPSGGGTTYAQKFQGRVTMTRDTSTNTVYMELISLRSDDTAVYYCARDGTPILPPSATTIGFDFWGQGTLVTVSS

SEQ ID NO: 21 - protein, 124 aa, synthetic construct
QVQLVESGGGVVQPGRSLRLSCAASGFTFSNYAMYWVRQAPGKGLEYMAFISYDGTKKDYADSVKGRFSISRDNSRETLWLQMNSLRAEDTAVYFCARDYLLIPRTASNAFDLWGQGTTVTVSS

SEQ ID NO: 22 - protein, 123 aa, synthetic construct
QVQLVQSGAEVKRPGASVKVSCKASGYTFISFAFHWVRQAPGQRLEWMGWINVANGNTKYSQNFQGRVTITRDTSAGTAYMELHSLRSEDTAVYFCATGDTGTHYHYYAMDVWGQGTTVTVSS

SEQ ID NO: 23 - protein, 121 aa, synthetic construct
QVTLKESGGGLVKPGGSLRLSCAASGFTFSSSTMTWVRQAPGKGLEWVSSISSSSSYIYYAASVKGRFTISRDNPKNSLYLQMNSLRAEDTALYYCARAFGGNSERGFDYWGQGTLVTVSS

SEQ ID NO: 24 - protein, 126 aa, synthetic construct
QVQLVQSGLEVKRPGASVKVSCKASGHTFTNYGINWIRQTPGQGLESLGWISASDGNINYARKFRGRVTMTTDTSTSTVYMELRSLRSDDTAVYYCARDRATFEGILAARDAFDIWGQGTMVTVSS

SEQ ID NO: 25 - protein, 126 aa, synthetic construct
QVQLVQSGPEVKKPGASVQVSCKAYGDTFMNLGFNWIRQAPGQGLESMGWISARDGNINYAPRFRGRVTMTTDTSTSTVSVELRNLRADDTAVYFCATDRATFGGILAARDAFHIWGQGTTVTVSS

SEQ ID NO: 26 - protein, 125 aa, synthetic construct
QVQLVQSGGGLIQPGGSLRLSCAASGFSVSSNYMTWVRQAPGKGLEWVSVIYAAGSTFYADSVKGRFTISRDNLTNTLSLQMNSLRADDTGVYFCARESVGDYNLWHYYYGMDVWGQGTTVTVSS

SEQ ID NO: 27 - protein, 121 aa, synthetic construct
EVQLVESGGGLVQPGGSLRLSCAASGFTFSSHWMSWVRQAPGKGPEWVANIKQDGSEKYYVDSVKGRFTISRDNVKNSLYLQMNTLRAEDTAIYYCVREIYYDDNSGFDYWGQGTLVTVSS

SEQ ID NO: 28 - protein, 124 aa, synthetic construct
QVQLVESGGGVVQPGRSLRLSCAASGFTFSNYAMYWVRQAPGKGLEYMAFISYDGTKKDYADSVKGRFSISRDNSRETLWLQMNSLRAEDTAVYFCARDYLLIPRTASNAFDLWGQGTMVTVSS

SEQ ID NO: 29 - protein, 124 aa, synthetic construct
EVQLVESGAEVKKPGASVKVSCKASGYTFTSYYIHWVRQAPGQGPEWMGIINASGGSTSYAQKFQGRVTMTRDTSTSTVYMELSSLRSEDTAVYYCARDGGGYYSSGRSWFDPWGQGTLVTVSS

SEQ ID NO: 30 - protein, 117 aa, synthetic construct
VQLQESGPGLVKPSQTLSLTCTVSGASIISSGDYYWSWIRQPPGKGLEWVGYIYYSGSTYYNPSLKSRVTISVDTSNNQFSLKLKSVTAADTAVYYCATLGPTVGYWGQGTLVTVSS

SEQ ID NO: 31 - protein, 119 aa, synthetic construct
QVQLVQSGAEVKKPGASVKVSCKASGYTFSSYGISWVRQAPGQGLEWMGWISLYNGNTNYAQNLQDRVTMTTGTSTSTAYLELRSLRSDDTAMYFCARVGYGINILDRWGQGTLVTVSS

SEQ ID NO: 32 - protein, 124 aa, synthetic construct
QVQLQESGGGLVQPGGSLRLSCAASGFTFSSYWMSWVRQAPGKGLEWVANIKQDGSEKYYVDSVKGRLTISRDNAKNSLYLQMNRLRAEDTAVYYCARDRGWLEPTPWDYFDYWGQGTLVTVSS

SEQ ID NO: 33 - protein, 122 aa, synthetic construct
QVQLVESGPGLVKPSQTLSLTCTVSGGSISSGGYYWSWIRQHPGKGLEWIGYIYYSGSTYYNPSLESRVTFSVDTSKDQFSLNLRSVTAADTAMYYCAPMTTVMSPWGFDPWGQGTLVTVSS

SEQ ID NO: 34 - protein, 119 aa, synthetic construct
QVQLVESGGGLVKPGGSLRLSCAASGFNFRSYGMNWVRQAPGKGLEWVSSISTSSSYKYYGDSVKGRFTISRDNAQQSVFLQMNSLRAEDTAIYFCARVDAEADALDCWGQGTLVTVSS

SEQ ID NO: 35 - protein, 119 aa, synthetic construct
QVQLQQWGPGLVKPSGTLSLTCGVSGDSITSNNWWTWVRQPPGKGLEWIGEIYHTGNTNYNPSLQGRVALSVDKSKNHFSLTLTSVTAADTAFYFCARSWELRRLYHDWGQGTLVTVSS

SEQ ID NO: 36 - protein, 122 aa, synthetic construct
QVQLQQSGPGLVKPSQTLSLTCAISGDSVSSNSAAWNWIRQSPSRGLEWLGRTYYRSKWYNDYAESVKSRITVTPDTSKNQFSLHLTSVTPEDTAVYYCGRDPPGDQTIDVWGQGTTVTVSS

SEQ ID NO: 37 - protein, 124 aa, synthetic construct
QVQLVQSGGGVVQPGRSLRLSCAASGFTFSNYAMYWVRQAPGKGLEYMAFISYDGTIKDYADSVKGRFSISRDNSRETVWLQMNSLRPEDTAVYFCARDYLLIPRTASNAFDLWGQGTTVTVSS

SEQ ID NO: 38 - protein, 124 aa, synthetic construct
QVQLVQSGGGVVQPGRSLRLSCVASGFTFSRYAMHWVRQAPGKGLEWVAVISYDGSKKEYVDSVKGRFAISRDNSKNMVYLQMHSLRAEDTAVYTCARDVILVPAAISDAFDIWGQGTMVTVSS

SEQ ID NO: 39 - protein, 120 aa, synthetic construct
EVQLVESGGGVVQPGRSLRLSCAASGFTFDSYGMHWVRQAPGKGLEWVAVIWYDGSDKYYADSVKGRFTISRDNSKNILYLQMNSLRAEDTAVYYCAKGPLQDGHYFDYWGQGTLVTVSS

SEQ ID NO: 40 - protein, 120 aa, synthetic construct
QVQLVQSGGEVKKPGASVKVSCKTSGYTFTNYGITWVRQAPGRGLEWMGWISVYTGYTNYAQKLQGRVTMTTDTSTRTAYMELRSLRSDDTALYYCARVQEGHSGRFDPWGQGTLVTSPQ

SEQ ID NO: 41 - protein, 116 aa, synthetic construct
QVQLVQSGAEVKKPGASVRVSCKASGYNFAHFDLHWVRQAPGQGPEWMGWIHAGTGDIKYSQKFQGRVIITRDTAASTAYMEVTSLRSEDMAVYYCASLPPQENLWGQGTTVTVSS

SEQ ID NO: 42 - protein, 122 aa, synthetic construct
EVQLVESGAEVKKPGESLKISCKGSGYSFTNYWVAWVRQVPGKGLEWMGTIYPDDSDTRYSPSFQGQVAISVDLSVNTAYVQWSSLKASDTAMYYCARPSPHNNAWKGFDIWGQGTTVTVSS

SEQ ID NO: 43 - protein, 120 aa, synthetic construct
QVQLVESGGGLAQPGGSLRLSCAASGFTFDNYAMAWVRQAPGKGLEWVSVISGSGTDTFYADSVNGRFTISRDNSKTTLYLQVSSLRAEDTALYYCARDATTPQGLFDYWGQGTLVTVSS

SEQ ID NO: 44 - protein, 120 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDSGVVVITWDYWGQGTLVTVSS

SEQ ID NO: 45 - protein, 120 aa, synthetic construct
QVQLVQSGAEVKKPGSSVKVSCKASGGTFSSYAISWVRQAPGQGLEWMGGIIPMVGSAKSAKKFQGRVTITADASTSTAYMELGSLTSDDTAVYYCAREEQWLIRAFDIWGQGTTVTVSS

SEQ ID NO: 46 - protein, 120 aa, synthetic construct; identical to SEQ ID NO: 45

SEQ ID NO: 47 - protein, 119 aa, synthetic construct
EVQLVESGGGLVQPGGSLRLSCAASGFTFSAYWMTWVRQAPGKGLEWVANIDKPGTKKYYVASIEGRFTISRDNAKNSLHLQMDYLRGEDTAVYYCVRDHRGDPDTSAWGQGTLVTVSS

SEQ ID NO: 48 - protein, 122 aa, synthetic construct
QVQLVQSGGGVVQAGRSLRLSCAASGFSFSNYGMHWVRQAPGKGLEWVAVIWYDGSKKYYAESVKGRFTISRDNSKSTVYLQMNSLRVEDTAVYYCARDGYRRLDYYGMDVWGQGTTVTVSS

SEQ ID NO: 49 - protein, 123 aa, synthetic construct
QITLKESGPGLVKPSQTLSLTCTVSGGSISSAEHYWSWIRQSPGKGLEWIGYIYYSGTTYYNPSLESRVTISLDTSKSQFSLKLSSLTSTDTAVYYCARASYCVGGSRPFDPWGQGTMVTVSS

SEQ ID NO: 50 - protein, 124 aa, synthetic construct
QVQLVESGGGLVQPGGSLRLSCAASGFTFTNYVMHWVRQAPGKGLEWVSGIGTVGDTYYLGSVKGRFTISRESAKKSLYLQMNSLRAGDTAVYYCARGGGGSDSLHWDTVIDDWGQGTLVTVSS

SEQ ID NO: 51 - protein, 117 aa, synthetic construct
QVTLKESGPGLVKPSQTLSLTCIVSGGSISGYFWSWIRQHPGKGLEWIGYVHYSGSTYYNPSLKSRVTISVDTSKNQFSLKLTSVTAADTAVYYCARASTSGGFDPWGQGTLVTVSQ

SEQ ID NO: 52 - protein, 119 aa, synthetic construct
QVQLVQSGAEVKKPGASVTVSCKASGYSFRSYHVHWVRQAPGQGLEWVAWIDADAADTIYAQKFQGRVTVTRDTSTSTVHMVLSGLKSDDTAVYYCARAPTIAFYFDHWGLGTLVTVSS

SEQ ID NO: 53 - protein, 115 aa, synthetic construct
QVQLVQSGGGLVQPGGSLRLSCAASGFTLSDYWMNWVRQAPGRGLEWVASIKADGSEKYYVDSVTGRFSISRDSGKNSLYLQMNSLTAEDTAIYYCVRGLSGIDWGXGTTVTVSS

SEQ ID NO: 54 - protein, 107 aa, synthetic construct
DIQVTQSPSSLSASVGDRVTITCRASRGISNYLAWFQQKPGKAPKSLIYAASSLQGGVPPRFSGGGSGTDFTLTISSLQPEDFATYYCQQYFGFPLTFGGGTKVEIK

SEQ ID NO: 55 - protein, 107 aa, synthetic construct
EIVMTQSPSSLSASVGDRVTITCRASQDITKSVVWYQQRLGKAPKLLVYGTSRLASGVPSRFSGSGYGTDYSLTISSLQPEDFATYYCQQYYSNPLTFGGGTKVEIK

SEQ ID NO: 56 - protein, 107 aa, synthetic construct
EIVLTQSPSSLSASVGDRVTITCRASQDITNSLVWYQQRPGKAPNLLLYGASRLASGVPSRFSGSGSGTDYSLTISSLQPEDFATYYCQQYYRNPLTFGGGTKVDIK

SEQ ID NO: 57 - protein, 112 aa, synthetic construct
EIVMTQSPLSLPVTPGEPASISCRSSQSLLYSNGYNYLDWYLQKPGKSPQLLIYLGSNRASGVPDRISGSGSGTDFTLKISRVEAEDVGVYYCMQALQISYTFGQGTKVDIK

SEQ ID NO: 58 - protein, 106 aa, synthetic construct
DIVMTQTPSSLSASVGDRVTITCRASQDITKSIVWYQQKAGKAPKVLLYASRLQSGVPSRFSGSGSGTDYTLTISSLQPEDFATYYCQQYYSNPLTFGGGTKLEIK

SEQ ID NO: 59 - protein, 113 aa, synthetic construct
DIRLTQSPDSLAVSLGERATINCKSRQSVLYSSINKNYLAWYQQKPGQPPKLLIYWASTRESGVPDRFSGSGSGTDFTLTISSLQAEDVAVYYCQQYYSLPYSFGQGTKLEIK

SEQ ID NO: 60 - protein, 106 aa, synthetic construct
EIVTTQSPSSLSASVGDRVTITCRASQDITKSIVWYQQKAGKAPKVLLYASRLQSGVPSRFSGSGSGTDYTLTISSLQPEDFATYYCQQYYSNPLTFGGGTKVDIK

SEQ ID NO: 61 - protein, 107 aa, synthetic construct
EIVMTQSPATLSVSPGDRATLSCRASRSVTSNLAWYQHKPGQAPRLLIYGASTRATGIPARFSGSGSGTEFTLTISSLQSEDLAVYFCQQYNDWPRTFGQGTKVDIK

SEQ ID NO: 62 - protein, 107 aa, synthetic construct
DIVLTQTPSSLSASVGDRVTITCRASQDITNSLVWYQQRPGKAPNLLLYGASRLASGVPSRFSGSGSGTDYSLTISSLQPEDFATYYCQQYYRNPLTFGGGTKVEIK

SEQ ID NO: 63 - protein, 107 aa, synthetic construct
EIVMTQSPSSLSASVGDRATITCRASQGITNSLAWYQQRPGKAPNLLLYGASRLARGVPSRFSGSGSGTDYSLTISSLQPEDYATYYCQQYYNNPLTFGGGTKVDIK

SEQ ID NO: 64 - protein, 107 aa, synthetic construct
DIQVTQSPSTLSASVGDRVTITCRASQSVDNWLAWYQQKPGKAPKVVIWRVSALESGVSPRFSGSGSGTEFTLTINSLQPDDFATYFCQQYKTYDLTFGGGTKVDIK

SEQ ID NO: 65 - protein, 108 aa, synthetic construct
DIVLTQSPGTLSLSPGERATLSCRASQSISSSYIAWYQQRPGQAPRLLIYGASTRAAGIPGRFSGSGSGTDFTLTISRLEPEDFAVYFCQQYGTSPKTFGQGTKLEIK

SEQ ID NO: 66 - protein, 112 aa, synthetic construct
DIVLTQSPLSLPVTLGQSASISCRSSQGLVHSDGNSYLNWFHQRPGQSPRRLIYKVSFRDSGVPDRFSGSGSGTNFTLKISRVEAEDVGVYYCMQGTHWPPAFGQGTKXDIK

SEQ ID NO: 67 - protein, 107 aa, synthetic construct
DIRLTQSPSSLSASVGDRVTITCRASQGITNSLAWYQQRPGKAPNLLLYGASRLARGVPSRFSGSGSGTDYSLTISSLQPEDYATYYCQQYYNNPLTFGGGTKVEIK

SEQ ID NO: 68 - protein, 112 aa, synthetic construct
DIVMTQTPDSLAVSLGERATINCKSSQSVLYSSNNKNYLAWYQQKPGHPPKVLIYWASTRESGVPDRFSGSGSGTDFTLTISSLQAEDVAVYYCQQYYTAATFGGGTKLEIK

SEQ ID NO: 69 - protein, 107 aa, synthetic construct
EIVLTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKLEIK

SEQ ID NO: 70 - protein, 107 aa, synthetic construct
EIVMTQSPSSLSASVGDRVTITCRASQDITNSLVWYQQRPGKAPNLLLYGASRLASGVPSRFSGSGSGTDYSLTISSLQPEDFATYYCQQYYRNPLTFGGGTKVDIK

SEQ ID NO: 71 - protein, 107 aa, synthetic construct
DIRLTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKLEIK

SEQ ID NO: 72 - protein, 107 aa, synthetic construct
DIRLTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTRLEIK

SEQ ID NO: 73 - protein, 107 aa, synthetic construct
EIVMTQSPATLSVSPGERATLSCRASRSVTINLAWYQQKPGQAPRLLIYGASTRATGIPARFSGSGSGTEFTLTISSLQSEDLAVYYCQQYNDWPRTFGQGTKVEIK

SEQ ID NO: 74 - protein, 107 aa, synthetic construct
EIVLTQSPATLSVSPGESATLSCRTSRSVTSNLAWYQQKLGQAPRLLIYGASIRATGIPARFSGSGSGTEFTLSISSLQSEDLGVYYCQQYNDWPRTFGQGTKLEIK

SEQ ID NO: 75 - protein, 107 aa, synthetic construct
ETTLTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKVEIK

SEQ ID NO: 76 - protein, 113 aa, synthetic construct
DIRLTQSPDSLAVSLGERATFNCKSSQSVLYSSTNKNYLAWYQQKPGLPPKLLIYWASTRASGVPDRFSGSGSGTDFTLTISSLQPEDVAVYYCHQYYTTPLTFGGGTKLEIK

SEQ ID NO: 77 - protein, 107 aa, synthetic construct
DIRLTQTPSSLSASVGDRVTITCRASQDITNSLVWYQQRPGKAPNLLLYGASRLASGVPSRFSGSGSGTDYSLTISSLQPEDFATYYCQQYYRNPLTFGGGTKVEIK

SEQ ID NO: 78 - protein, 107 aa, synthetic construct
DIQVTQSPSSLSASVGDRVTITCRASQDISNSLVWYQQKAGQAPKILLYGSSRLQSGVPSRFSGSGSGTDYTLTISGLLPEDFATYYCQQYYNNPLTFGGGTKVDIK

SEQ ID NO: 79 - protein, 107 aa, synthetic construct
EIVLTQSPSSLSASVGDRVTITCRASQDISNSLVWYQQKAGQAPKILLYGSSRLQSGVPSRFSGSGSGTDYTLTISGLLPEDFATYYCQQYYNNPLTFGGGTKLEIK

SEQ ID NO: 80 - protein, 107 aa, synthetic construct
ETAFTQSPATLSLSPGERVTLSCRASQSVGSSLAWYQHKPGQAPRLLIYDASKRATGIPARFSGSGSGTDFTLTISSLEPEDFAVYYCQQCSNWPLTFGGGTKVEIK

SEQ ID NO: 81 - protein, 107 aa, synthetic construct
DIQMTQSPSSLSASVGDRVTITCRASLSISSYVNWFQQKPGKAPKFLIYAASSLQTGVPSRFSGGGSGTDFTLTINSLQPEDFATYYCQQSYSTPYTFGQGTKVDIK

SEQ ID NO: 82 - protein, 113 aa, synthetic construct
DIVMTQSPDSLAVSLGERATINCKSSLSVLYSSNNRNYLAWYQQKLGQPPKLLFYWASTRESGVPDRFSGSGSGTDFTLTISNLQAEDVAVYYCQQYYTAPWTFGQGTKVEIK

SEQ ID NO: 83 - protein, 107 aa, synthetic construct
DIQLTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKLEIK

SEQ ID NO: 84 - protein, 107 aa, synthetic construct
DIRMTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKVEIK

SEQ ID NO: 85 - protein, 107 aa, synthetic construct
DIRVTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKVDIK

SEQ ID NO: 86 - protein, 107 aa, synthetic construct
DIRVTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKLEIK

SEQ ID NO: 87 - protein, 107 aa, synthetic construct
DIQVTQSPSSLSASVGDRVTITCQASQDISNYLNWYQQKPGKAPKPLIYDASNLETGVPSRFGGSGSGTDFTFTISSLQPEDIATYYCQQYDNLRLTFGGGTKVEIK

SEQ ID NO: 88 - protein, 113 aa, synthetic construct
EIVLTQSPLSLPVTLGQPASISCRSSQSLVHRDGNTYLTWFQQRPGQSPRRLVHKVSNRDSGVPDRFSGSGSGTYFTLRISRVEAEDVGVYFCMQGTHRPYYNFGQGTKLEIK

SEQ ID NO: 89 - protein, 107 aa, synthetic construct
EIRVTQSPSSLSASVGDRVTITCRASQDITKSLVWYQQRPGKAPNLLLYGASRLASGVPSRFSGSGSGTDYRLTISSLQPEDFATYYCQQYHRYPLTFGGGTKVDIK

SEQ ID NO: 90 - protein, 113 aa, synthetic construct
ETTLTQSPDSLAVSLGERATINCKSSQSVLYSFNSQNYLAWYQQKPGQPPKLLIYWASTRESGVPVRFSGSGSGTDFTLTISSLQAEDVAVYYCQQYVSTPLTFGQGTKVDIK

SEQ ID NO: 91 - protein, 107 aa, synthetic construct
EIVLTQSPSSLSASVGDRVTITCRASQGITNSLVWYQQKPGKAPKLLVYGTSRLESGVPSRFSGSGSGTDYTLTISSLQPEDFATYYCQQYYNYPLTFGGGTKVDIK

SEQ ID NO: 92 - protein, 113 aa, synthetic construct
DIVMTQSPDSLALSLGERATINCKSSQSVLYSSNNKNYLAWYQQKPGQPPKLLIYWASTRESGVPDRFSGSGAGTDFTLTISSLQAEDVAVYYCQQYYGFPFTFGPGTKVEIK

SEQ ID NO: 93 - protein, 107 aa, synthetic construct
DIQVTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKVDIK

SEQ ID NO: 94 - protein, 107 aa, synthetic construct
DIRMTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKVDIK

SEQ ID NO: 95 - protein, 113 aa, synthetic construct
EIVMTQSPDSLAVSLGERATINCKSSQSVLYSSNNKNYLSWYQQKPGQPPKLLIFWASTRESGVPDRFSGSGSETDFTLTISSLQAEDVAVYYCQQYFDTPLTFGGGTKVEIK

SEQ ID NO: 96 - protein, 107 aa, synthetic construct
ETTLTQSPATLSLSPGERATLSCRATQSIINYLAWYQQKHGQPPRLLIYDASHRATGIPARFSGSGSGTDFTLTISSLEPEDFAVYYCQQRGNWPPTFGGGTKVEIK

SEQ ID NO: 97 - protein, 107 aa, synthetic construct
DIQVTQSPSSLSPSVGDRVTITCQASQDISNYLNWYQQKPGKAPKLLIYDASNLETGVPSRFGGSGSGTDFTFTISSLQPEDIATYYCQQYDNLRLTFGGGTKVDIK

SEQ ID NO: 98 - protein, 107 aa, synthetic construct
DIVMTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKVDIK

SEQ ID NO: 99 - protein, 112 aa, synthetic construct
EIVMTQSPLFLPVTLGQPASISCRSSQSLVHSDGNTYLSWFQQRPGQSPRRLIYKVSNRDSGVPDRFGGSGSGTDFTLKISRVEAEDVGVYYCMQATHWPFTLGPGTKLEIK

SEQ ID NO: 100 - protein, 107 aa, synthetic construct
EIVMTQSPATLSLSPGERATLSCWASQNIMGYLAWYQQKPGQAPRLLIYDAFNRATGVPAKFSGSGSGTDFTLTISSLEPEDFAVYYCQQRSSWPLTFGGGTKVEIK

SEQ ID NO: 101 - protein, 108 aa, synthetic construct
EIVMTQSPGTLSLSPGERATLSCRASQSVHNTFLAWYQQRFGQAPRLLIYGASSRATGIPDRFSGSGSGTDFTLTINRLEAEDFAVYYCQQYSNPPFTFGGGTKVDIK

SEQ ID NO: 102 - protein, 107 aa, synthetic construct
DIVMTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKLEIK

SEQ ID NO: 103 - protein, 108 aa, synthetic construct
EIVLTQSPGTLSLSPGDRAALTCRTSQSISYRQLAWYQHKPGQPPRLLIHGASNRATGIPDRFSGSGSGTDFTLTISRLEPEDFAVFYCQQYSTSPPTFGGGTKVEIK

SEQ ID NO: 104 - protein, 112 aa, synthetic construct
EIVLTQSPVSLSVTLGQPASISCRSTHSLDYSDGNTYLNWFHQRPGHSPRRLIYTVSDRDSGVPDRFSGSGSGTDFTLKISRVEAEDVGVYYCMQGTHWPYTFGXGXKVEIK

SEQ ID NO: 105 - protein, 112 aa, synthetic construct
DIVLTQSPVSLSVTLGQPASISCRSTHSLDYSDGNTYLNWFHLRPGHSPRRLIYTVSDRDSGVPDRFSGSGSGTDFTLKISRVEAEDVGVYYCMQGTHWPYTFGQGTKVEIK

SEQ ID NO: 106 - protein, 112 aa, synthetic construct
EIVMTQSPLSLPVTPGEPASISCRSGQSLLHSNGYNYLDWYLQKPGQSPQLLIYLGSNRASGVPDRFSGSGSGTDFTLKISRVEADDVGVYYCMQALQTPYTFGQGTKVDIK

SEQ ID NO: 107 - protein, 127 aa, synthetic construct
QVTLKESGAAVKKPGSSVTVSCKASGGSFSTYAMTWVRQAPGQGLEWMGGITPVFGIVDYAQKFQGRVTITADASTSTAYMELTSLRSEDSAVYYCARIGYCTAGDCSIPRGAFDIWGQGTMVTVSS

SEQ ID NO: 108 - protein, 128 aa, synthetic construct
EVQLLESGGGVVQPGGSLRLSCAASGFSFSDYWMHWVRQVPGQGLEWVSRIETDGSSTTYVDSVKGRFTISRDNANNTLFLQMNSLRAEDTAVYYCARDEGPFYYDFSSGFSDGMDVWGQGTTVTVSS

SEQ ID NO: 109 - protein, 121 aa, synthetic construct
QVQLVQSGAEVKKPGASVKVSCKASGYSFISYYMHWVRQAPGQGLEWMGIINPSGGSTSYAQNFQGRVTMTSDTSTSTVYMELSSLRSEDTAVYYCARDRLRKDAYGMDVWGQGTTVTVSS

SEQ ID NO: 110 - protein, 127 aa, synthetic construct
QVQLVESGAEVKEPGASVKVSCKASGYTFTSFTIHWVRQAPGQSLEWMGWINAGNGYTKYSQRFQDRLTITSDTSANTAYMDLRSLGSEDTALYYCARGRGDRRIVVLQPFEALDFWGQGTTVTVSS

SEQ ID NO: 111 - protein, 124 aa, synthetic construct
QVQLVQSGAEVRKPGSSVKVSCKASGGTFHSYTINWVRQAPGQGLQVLGGILPVFGTTNYAQKFQDRVIITADASTSTAYMELSSLRSEDTAVYYCAKLAGQPIVGSDYYYDSWGQGTLVTVSS

SEQ ID NO: 112 - protein, 108 aa, synthetic construct
DIXMTQSPGTLSLSPGERAALSCRASQSVSSTYLAWYQQKPGQAPRLLIYGTSSRATGIPDRFSGSGSGTDFTLTISRLEPEDFAVYYCQQYGSSALTFGGGTKVDIK

SEQ ID NO: 113 - protein, 107 aa, synthetic construct
DIQVTQSPSSLSASVGDRVTFTCRASQSVDTYLNWYQQKPGKAPKLLIYTASSLQLGVPSRFSGRGAGTEFTLTISSLQPEDFATYYCQQTYNTPHTFGQGTKVEIK

SEQ ID NO: 114 - protein, 107 aa, synthetic construct
DIQLTQSPSSLSASVGDRVTITCRASQSISTYLNWYQQKPGRAPNLLIYDASSVQSGVPSRFSGSGSGTDFTLTISSLQPDDFVTYYCQQSYSSPRTFGQGTKVDIK

SEQ ID NO: 115 - protein, 113 aa, synthetic construct; identical to SEQ ID NO: 90

SEQ ID NO: 116 - protein, 107 aa, synthetic construct
ETMMTQTPASLSVSPGERAILSCWASQSVGRNLAWYQHKPGQAPRLLVYAASARATGVPARFSGGGSGTDFTLTISSLQSEDFAVYYCQQYDKWPGTFGQGTKVDIK

SEQ ID NO: 117 - protein, 125 aa, synthetic construct
QVQLVQSGGGVVQPGRSLRLSCAVSGFTFSPYAMHWVRQAPGKGLEWVAYISSYEGTTNDYADSVKGRFTISRDNSKNTLYLQMNSLRPEDTALYYCARDLFLISRSSSYAFDLWGQGTLVTVSS

SEQ ID NO: 118 - protein, 125 aa, synthetic construct; identical to SEQ ID NO: 117

SEQ ID NO: 119 - protein, 125 aa, synthetic construct
QVQLVQSGGGVVQPGRSLRLSCAVSGFTFSPYAMHWVRQTPGKGLEWVAYISSYEGTTNDYADSVKGRFTISRDNSKNTLYLQMNSLRPEDTALYYRARDLFLISRSSSYAFDLWGQGTMVTVSS

SEQ ID NO: 120 - protein, 125 aa, synthetic construct; identical to SEQ ID NO: 119

SEQ ID NO: 121 - protein, 125 aa, synthetic construct
EVQLVESGGGVVQPGRSLRLSCAVSGFTFSPYAMHWVRQAPGKGLEWVAYISSYEGTTNDYADSVKGRFTISGDNSKNTLYLQMNSLRPEDTALYYCARDLFLISRSSSYAFDLWGQGTTVTVSS

SEQ ID NO: 122 - protein, 125 aa, synthetic construct
QVQLVESGGGVVQPGRSLRLSCAVSGFTFSPYAMHWVRQAPGKGLEWVAYISSYEGTTNDYADSVKGRFTISRDNSKNTLYLQMNSLRPEDTALYYCARDLFLISRSSSYAFDLWGQGTMVTVSS

SEQ ID NO: 123 - protein, 124 aa, synthetic construct; identical to SEQ ID NO: 32

SEQ ID NO: 124 - protein, 124 aa, synthetic construct; identical to SEQ ID NO: 37

SEQ ID NO: 125 - protein, 120 aa, synthetic construct; identical to SEQ ID NO: 44

SEQ ID NO: 126 - protein, 107 aa, synthetic construct
EIVMTQSPSSLSASVGDRVTITCRASQGIRNDLGWYQQKPGKAPKRLIYAASSLQSGVPSRFSGSGSGTEFTLTISSLQPEDFATYYCLQHNSYPYTFGQGTRLEIK

SEQ ID NO: 127 - protein, 107 aa, synthetic construct; identical to SEQ ID NO: 126

SEQ ID NO: 128 - protein, 107 aa, synthetic construct; identical to SEQ ID NO: 126

SEQ ID NO: 129 - protein, 107 aa, synthetic construct; identical to SEQ ID NO: 126

SEQ ID NO: 130 - protein, 107 aa, synthetic construct; identical to SEQ ID NO: 78

SEQ ID NO: 131 - protein, 107 aa, synthetic construct; identical to SEQ ID NO: 79

SEQ ID NO: 132 - protein, 107 aa, synthetic construct; identical to SEQ ID NO: 78

SEQ ID NO: 133 - protein, 107 aa, synthetic construct; identical to SEQ ID NO: 79

SEQ ID NO: 134 - protein, 107 aa, synthetic construct; identical to SEQ ID NO: 100

SEQ ID NO: 135 - protein, 107 aa, synthetic construct
EIVMTQSPSSLSASVGDRVTITCQASQDISNYLNWYQQKPGKAPKLLIYDASNLETGVPSRFGGSGSGTDFTFTISSLQPEDIATYYCQQYDNLRLTFGGGTKVDIK

SEQ ID NO: 136 - protein, 125 aa, synthetic construct
EVQLLESGAEVKKPGSSVKVSCQASGGTFSTYAVSWVRQAPGQGLEWMGGIIPIFGTANYAQKFQGRVTITADKSTNTAYMEMSRLRSEDTAVYYCARVAGRGTVVTPWNHFDYWGQGTLVTVSS

SEQ ID NO: 137 - protein, 125 aa, synthetic construct
QVQLVQSGAEVKKPGSSVKVSCQASGGTFSTYAVSWVRQAPGQGLEWMGGIIPIFGTANYAQKFQGRVTITADKSTNTAYMEMSRLRSEDTAVYYCARVAGRGTVVTPWNHFDYWGQGTLVTVSS

SEQ ID NO: 138 - protein, 128 aa, synthetic construct
QVQLVESGAEVRKPGSSVKVSCKASGGTFSSYAISWVRQAPGQGLEWMGGIIPIFGTPNYAQRFQDRVTITADASTSTAYMELSSLRSDDTAVYYCAREGYCLGSTCHLSGGGAFDIWGLGTTVTVSS

SEQ ID NO: 139 - protein, 122 aa, synthetic construct
QVTLKESGAEVKKPGSSVKVSCKASEGTIRTYAVTWLRQAPGQRLEWMGGIIPIFGKVNHAERFQGRVAITADELTGTVYMELSSLTSQDTAMYFCARDLMLANSPPAFDLWGQGTLVTVSS

SEQ ID NO: 140 - protein, 128 aa, synthetic construct
EVQLVESGAEVKKPGASVRASCKTSGYPFTSYYIHWVRQAPGQGLEWMGMINPSGGSTRYAQKFQGRVTMTSDTSTSTVYMEMSSLRSVDTAVYYCARSSPTGYSTNRGRGYSGIDVWGQGTTVTVSS

SEQ ID NO: 141 - protein, 106 aa, synthetic construct
DIQLTQSPATLSVSPGERATLSCRASQSVSGNLAWYQHKPGQAPRLLIYGASTRATGIPARFSGSGSGTEFTLTISCLQSEDFAVYYCQQYNNWVTFGGGTKVEIK

SEQ ID NO: 142 - skipped

SEQ ID NO: 143 - protein, 108 aa, synthetic construct
DIVMTQSPSTLSASVGDRVTITCRASQSINSWSAWYQQKPGKAPKPLIYQASSLESGVPSRFSGSGSGTEFTLTISSLQPDDFATYYCQQYNSYSSYTFGQGTKVDIK

SEQ ID NO: 144 - protein, 108 aa, synthetic construct
DIVMTQTPGTLSLSPGERATLSCRASQSVSGNYLAWYQQKPGQAPRLLIYGASSRATGIPDRFSDSGSGTDFTLTISRLEPEDFAVYYCQQYGSSPLTFGGGTKVDIK

SEQ ID NO: 145 - protein, 107 aa, synthetic construct
DIQMTQSPSSLSASVGDRVTITCKASQDVTTAVAWYQQKPGKAPKLLIYWASTRHTGVPSRFSGSGSGTDFTLTISSLQPEDFATYYCQQHYSTPLTFGQGTKVEIK

SEQ ID NO: 146 - protein, 107 aa, synthetic construct
DIVMTQSPATLSVSPGEGATLSCRASQSVGSNLAWYQHKPGQPPRLLIYGASARATSIPAKFSGSGSGTDFTLTISSLQSEDFAVYYCQQYNNWPLTFGGGTKVDIK

SEQ ID NO: 147-152 - skipped (6 entries)

SEQ ID NO: 153 - protein, 117 aa, synthetic construct
QVQLVQSGGGLIQPGESLRLSCAVSGFTVNNNYISWVRQAPGKALEWVSVIYHGARAYYADSVKGRFTISIDNSKNTLYLQMNSLRAEDTAVYYCARDRGSGDMDAWGQGTTVTVSS

SEQ ID NO: 154-200 - skipped (47 entries)

SEQ ID NO: 201 - DNA, 2715 bp, Human herpesvirus 1
  source 1..2715: mol_type = genomic DNA
atgcgccagggcgcccccgcgcgggggtgccggtggttcgtcgtatgggcgctcttggggttgacgctgggggtcctggtggcgtcggcggctccgagttcccccggcacgcctggggtcgcggccgcgacccaggcggcgaacgggggccctgccactccggcgccgcccgcccttggcgccgccccaacgggggacccgaaaccgaagaagaacaaaaaaccgaaaaacccaacgccgccacgccccgccggcgacaacgcgaccgtcgccgcgggccacgccaccctgcgcgagcacctgcgggacatcaaggcggagagcaccgatgcaaacttttacgtgtgcccaccccccacgggcgccacggtggtgcagttcgagcagccgcgccgctgcccgacccggcccgagggtcagaactacacggagggcatcgcggtggtcttcaaggagaacatcgccccgtacaagttcaaggccaccatgtactacaaagacgtcaccgtttcgcaggtgtggttcggccaccgctactcccagtttatggggatctttgaggaccgcgcccccgtccccttcgaggaggtgatcgacaagatcaacgccaagggggtctgtcggtccacggccaagtacgtgcgcaacaacctggagaccaccgcgtttcaccgggacgaccacgagaccgacatggagctgaaaccggccaatgccgcgacccgcacgagccggggctggcacaccaccgacctcaagtacaacccctcgcgggtggaggcgttccaccggtacgggacgacggtaaactgcatcgtcgaggaggtggacgcgcgctcggtgtacccgtacgacgagtttgtgctggcgactggcgactttgtgtacatgtccccgttttacggctaccgggaggggtcgcacaccgaacacaccagctacgccgccgaccgcttcaagcaggtcgacggcttctacgcgcgcgacctcaccaccaaggcccgggccacggcgccgaccacccggaacctgctcacgacccccaagttcaccgtggcctgggactgggtgccaaagcgcccgtcggtctgcaccatgaccaagtggcaggaggtggacgagatgctgcgctccgagtacggcggctccttccgattctcctccgacgccatatccaccaccttcaccaccaacctgaccgagtacccgctctcgcgcgtggacctgggggactgcatcggcaaggacgcccgcgacgccatggaccgcatcttcgcccgcaggtacaacgcgacgcacattaaggtgggccagccgcagtactacctggccaatgggggctttctgatcgcgtaccagccccttctcagcaacacgctcgcggagctgtacgtgcgggaacacctccgagagcagagccgcaagcccccaaaccccacgcccccgccgcccggggccagcgccaacgcgtccgtggagcgcatcaagaccacctcctccatcgagttcgcccggctgcagtttacgtacaaccacatacagcgccatgtcaacgatatgttgggccgcgttgccatcgcgtggtgcgagctgcagaatcacgagctgaccctgtggaacgaggcccgcaagctgaaccccaacgccattgcatcggccaccgtgggccggcgggtgagcgcgcggatgctcggcgacgtgatggccgtctccacgtgcgtgccggtcgccgcggacaacgtgatcgtccaaaactcgatgcgcatcagctcgcggcccggggcctgctacagccgccccctggtcagctttcggtacgaagaccagggcccgttggtcgaggggcagctgggggagaacaacgagctgcggctgacgcgcgatgcgatcgagccgtgcaccgtgggacaccggcgctacttcaccttcggtgggggctacgtgtacttcgaggagtacgcgtactcccaccagctgagccgcgccgacatcaccaccgtcagcaccttcatcgacctcaacatcaccatgctggaggatcacgagtttgtccccctggaggtgtacacccgccacgagatcaaggacagcggcctgctggactacacggaggtccagcgccgcaaccagctgcacgacctgcgctttgccgacatcgacacggtcatccacgccgacgccaacgccgccatgttcgcgggcctgggtgcgtttttcgaggggatgggcgacctggggcgcgcggtcggcaaggtggtgatgggcatcgtgggcggcgtggtatcggccgtgtcgggcgtgtcctccttcatgtccaacccctttggggcgctggccgtgggtctgttggtcctggccggcctggcggcggccttcttcgcctttcgctacgtcatgcggctgcagagcaaccccatgaaggccctgtacccgctaaccaccaaggagctcaagaaccccaccaacccggacgcgtccggggagggcgaggagggcggcgactttgacgaggccaagctagccgaggcccgggagatgatacggtacatggccctggtgtcggccatggagcacacggaacacaaggccaagaagaagggcacgagcgcgctgcttagcgccaaggtcaccgacatggtcatgcgcaagcgccgcaacaccaactacacccaagttcccaacaaagacggtgacgccgacgaggacgacctgtga

SEQ ID NO: 202 - protein, 904 aa, Human alphaherpesvirus 1
MRQGAPARGCRWFVVWALLGLTLGVLVASAAPSSPGTPGVAAATQAANGGPATPAPPALGAAPTGDPKPKKNKKPKNPTPPRPAGDNATVAAGHATLREHLRDIKAESTDANFYVCPPPTGATVVQFEQPRRCPTRPEGQNYTEGIAVVFKENIAPYKFKATMYYKDVTVSQVWFGHRYSQFMGIFEDRAPVPFEEVIDKINAKGVCRSTAKYVRNNLETTAFHRDDHETDMELKPANAATRTSRGWHTTDLKYNPSRVEAFHRYGTTVNCIVEEVDARSVYPYDEFVLATGDFVYMSPFYGYREGSHTEHTSYAADRFKQVDGFYARDLTTKARATAPTTRNLLTTPKFTVAWDWVPKRPSVCTMTKWQEVDEMLRSEYGGSFRFSSDAISTTFTTNLTEYPLSRVDLGDCIGKDARDAMDRIFARRYNATHIKVGQPQYYLANGGFLIAYQPLLSNTLAELYVREHLREQSRKPPNPTPPPPGASANASVERIKTTSSIEFARLQFTYNHIQRHVNDMLGRVAIAWCELQNHELTLWNEARKLNPNAIASATVGRRVSARMLGDVMAVSTCVPVAADNVIVQNSMRISSRPGACYSRPLVSFRYEDQGPLVEGQLGENNELRLTRDAIEPCTVGHRRYFTFGGGYVYFEEYAYSHQLSRADITTVSTFIDLNITMLEDHEFVPLEVYTRHEIKDSGLLDYTEVQRRNQLHDLRFADIDTVIHADANAAMFAGLGAFFEGMGDLGRAVGKVVMGIVGGVVSAVSGVSSFMSNPFGALAVGLLVLAGLAAAFFAFRYVMRLQSNPMKALYPLTTKELKNPTNPDASGEGEEGGDFDEAKLAEAREMIRYMALVSAMEHTEHKAKKKGTSALLSAKVTDMVMRKRRNTNYTQVPNKDGDADEDDL

SEQ ID NO: 203 - DNA, 1185 bp, Human herpesvirus 1
  source 1..1185: mol_type = genomic DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacataccgtcgatccaggacgccgcgacgccttaccatcccccggccaccccgaacaacatgggcctgatcgccggcgcggtgggcggcagtctcctggcagccctggtcatttgcggaattgtgtactggatgcgccgccgcactcaaaaagccccaaagcgcatacgcctcccccacatccgggaagacgaccagccgtcctcgcaccagcccttgttttactag

SEQ ID NO: 204 - protein, 394 aa, Human alphaherpesvirus 1
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMRRRTQKAPKRIRLPHIREDDQPSSHQPLFY

(19) United States
(12) Patent Application Publication
(10) Pub. No.: US 2024/0352094 A1
(43) Pub. Date: Oct. 24, 2024

(54) COMPOSITIONS AND METHODS FOR THE TREATMENT OF HERPES SIMPLEX VIRUS INFECTION

(71) Applicant: Albert Einstein College of Medicine, Bronx, NY (US)

(72) Inventors: Jonathan R. Lai, Dobbs Ferry, NY (US); Karen Tong, Bronx, NY (US); Betsy C. Herold, Rowayton, CT (US); Aakash M. Mahant, Bronx, NY (US)

(21) Appl. No.: 18/580,318

(22) PCT Filed: Jul. 20, 2022
(86) PCT No.: PCT/US22/37706

§ 371 Date: Jan. 18, 2024

Related U.S. Application Data
(60) Provisional application No. 63/223,898, filed on Jul. 20, 2021.

(51) Int. Cl. C07K 16/08; A61K 39/00; A61P 31/22; A61P 37/04

(52) U.S. Cl. CPC C07K 16/087; A61P 31/22; A61P 37/04; A61K 2039/505

(57) ABSTRACT

Provided herein are compositions and methods for the treatment of Herpes Simplex Virus infections.

### CROSS-REFERENCE TO RELATED APPLICATIONS

This application claims the benefit of U.S. Provisional Application No. 63/223,898, filed on Jul. 20, 2021, the entire contents of which are incorporated herein in their entirety by this reference.

### STATEMENT OF GOVERNMENT SUPPORT

This invention was made with government support under grant numbers NIH R01-AI125462 awarded by the National Institutes of Health. The government has certain rights in the invention.

### BACKGROUND OF THE INVENTION

The disclosures of all publications, patents, patent application publications and books referred to herein, are hereby incorporated by reference in their entirety into the subject application to more fully describe the art to which the subject invention pertains.

Herpes simplex viruses (HSV-1 and HSV-2) are among the most prevalent infections worldwide and the leading cause of oral and genital mucosal disease. Much attention has focused on prevention of genital herpes because of its association with an increased risk of HIV acquisition and transmission and risk of transmission to neonates. Although HSV-2 dominates globally, in the United States and other developed countries, HSV-1 has emerged as the more common cause of genital infections. In the United States among persons 14-49 years of age, the seroprevalence is estimated to be 47.8% and 11.9% for HSV-1 and HSV-2¹, respectively, with an additional 570,000 incident infections attributable to HSV-2 in 2018².

Both serotypes establish lifelong infection in sensory neurons with symptomatic or more often subclinical episodes of reactivation. Even in the absence of symptoms, reactivating virus can be shed in genital secretions and transmitted to sexual partners or neonates.³ Thus, while the immune response to natural infection prevents viral dissemination, it does not eliminate the virus and the correlates of immune protection have not been fully defined. Serum analysis of HSV-2 seropositive patients has shown that the predominant antibody response is the generation of neutralizing antibodies targeting glycoprotein D (gD) and glycoprotein B (gB), which are both required for viral entry and spread. gD is highly immunogenic and elicits a strong neutralizing response. This observation provided the rationale for vaccine designs but candidate vaccines that elicit a neutralizing response to gD alone or gD and gB have failed in clinical trials. Moreover, high neutralizing titers do not eradicate viral shedding in natural human infection.

Recently, a vaccine candidate in which envelope glycoprotein D (gD), an HSV protein involved in the fusion of the viral membrane with a host cell membrane, was genetically deleted (ΔgD-2) was shown to provide complete protection in mice challenged with 10-100 times the lethal dose of HSV-1 or HSV-2 clinical isolates and prevented the establishment of latency in murine models. The vaccine elicited high titer Abs that passively protected naive mice from infection. Notably, the Abs had little neutralizing activity but engaged Fc receptors to mediate antibody dependent cellular cytotoxicity (ADCC) and phagocytosis (ADCP): the Fc component also bound complement to induce complement dependent cytolysis (CDC). One of the targets of the ADCC/ADCP and CDC mediating Abs is envelope glycoprotein B (gB). However, none of the resulting antibodies exhibited neutralizing activity.

There is an urgent need for effective therapies for treating HSV infections that initiate or mediate ADCC, ADCP, and CDC and have neutralizing activity.

### SUMMARY OF THE INVENTION

The present invention is based, at least in part, on the discovery, characterization, and development of monoclonal antibodies that have both neutralizing and antibody-dependent cellular cytotoxicity.

In one aspect, a recombinant monoclonal antibody is provided that specifically binds to herpes simplex virus glycoprotein B (gB) comprising a heavy chain comprising an amino acid sequence having at least 95% identity to an amino acid sequence listed in Table 2 and a light chain comprising an amino acid sequence having at least 95% identity to an amino acid sequence listed in Table 2.

In some embodiments, the heavy chain of the monoclonal antibody comprises an amino acid sequence has at least 95% identity to SEQ ID NO: 1 and the light chain comprises an amino acid sequence has at least 95% identity to SEQ ID NO: 54. In some embodiments, the heavy chain of the monoclonal antibody chain comprises an amino acid sequence has at least 95% identity to SEQ ID NO: 2 and the light chain comprises an amino acid sequence has at least 95% identity to SEQ ID NO: 55. In some embodiments, the heavy chain of the monoclonal antibody comprises an amino acid sequence has at least 95% identity to SEQ ID NO: 3 and the light chain comprises an amino acid sequence has at least 95% identity to SEQ ID NO: 56. In some embodiments, the heavy chain of the monoclonal antibody comprises an amino acid sequence has at least 95% identity to SEQ ID NO: 4 and the light chain comprises an amino acid sequence has at least 95% identity to SEQ ID NO: 57. In some embodiments, the heavy chain of the monoclonal antibody comprises an amino acid sequence has at least 95% identity to SEQ ID NO: 5 and the light chain comprises an amino acid sequence has at least 95% identity to SEQ ID NO: 58. In some embodiments, the heavy chain of the monoclonal antibody comprises an amino acid sequence has at least 95% identity to SEQ ID NO: 6 and the light chain comprises an amino acid sequence has at least 95% identity to SEQ ID NO: 59. In some embodiments, the heavy chain of the monoclonal antibody comprises an amino acid sequence has at least 95% identity to SEQ ID NO: 7 and the light chain comprises an amino acid sequence has at least 95% identity to SEQ ID NO: 60. In some embodiments, the heavy chain of the monoclonal antibody comprises an amino acid sequence has at least 95% identity to SEQ ID NO: 8 and the light chain comprises an amino acid sequence has at least 95% identity to SEQ ID NO: 61. In some embodiments, the heavy chain of the monoclonal antibody comprises an amino acid sequence has at least 95% identity to SEQ ID NO: 9 and the light chain comprises an amino acid sequence has at least 95% identity to SEQ ID NO: 62. In some embodiments, the heavy chain of the monoclonal antibody comprises an amino acid sequence has at least 95% identity to SEQ ID NO: 10 and the light chain comprises an amino acid sequence has at least 95% identity to SEQ ID NO: 63. In some embodiments, the heavy chain of the monoclonal antibody comprises an amino acid sequence has at least 95% identity to SEQ ID NO: 11 and the light chain comprises an amino acid sequence has at least 95% identity to SEQ ID NO: 64. In some embodiments, the heavy chain of the monoclonal antibody comprises an amino acid sequence has at least 95% identity to SEQ ID NO: 16 and the light chain comprises an amino acid sequence has at least 95% identity to SEQ ID NO: 65. In some embodiments, the heavy chain of the monoclonal antibody comprises an amino acid sequence has at least 95% identity to SEQ ID NO: 17 and the light chain comprises an amino acid sequence has at least 95% identity to SEQ ID NO: 66. In some embodiments, 95% identity to SEQ ID NO: 18 and the light chain comprises an amino acid sequence has at least 95% identity to SEQ ID NO: 67. In some embodiments, the heavy chain of the monoclonal antibody comprises an amino acid sequence has at least 95% identity to SEQ ID NO: 19 and the light chain comprises an amino acid sequence has at least 95% identity to SEQ ID NO: 68. In some embodiments, the heavy chain of the monoclonal antibody comprises an amino acid sequence has at least 95% identity to SEQ ID NO: 20 and the light chain comprises an amino acid sequence has at least 95% identity to SEQ ID NO: 69. In some embodiments, the heavy chain of the monoclonal antibody comprises an amino acid sequence has at least 95% identity to SEQ ID NO: 21 and the light chain comprises an amino acid sequence has at least 95% identity to SEQ ID NO: 70. In some embodiments, the heavy chain of the monoclonal antibody comprises an amino acid sequence has at least 95% identity to SEQ ID NO: 22 and the light chain comprises an amino acid sequence has at least 95% identity to SEQ ID NO: 71. In some embodiments, the heavy chain of the monoclonal antibody comprises an amino acid sequence has at least 95% identity to SEQ ID NO: 23 and the light chain comprises an amino acid sequence has at least 95% identity to SEQ ID NO: 72. In some embodiments, the heavy chain of the monoclonal antibody comprises an amino acid sequence has at least 95% identity to SEQ ID NO: 24 and the light chain comprises an amino acid sequence has at least 95% identity to SEQ ID NO: 73. In some embodiments, the heavy chain of the monoclonal antibody comprises an amino acid sequence has at least 95% identity to SEQ ID NO: 25 and the light chain comprises an amino acid sequence has at least 95% identity to SEQ ID NO: 74. In some embodiments, the heavy chain of the monoclonal antibody comprises an amino acid sequence has at least 95% identity to SEQ ID NO: 26 and the light chain comprises an amino acid sequence has at least 95% identity to SEQ ID NO: 75. In some embodiments, the heavy chain of the monoclonal antibody comprises an amino acid sequence has at least 95% identity to SEQ ID NO: 27 and the light chain comprises an amino acid sequence has at least 95% identity to SEQ ID NO: 76. In some embodiments, the heavy chain of the monoclonal antibody comprises an amino acid sequence has at least 95% identity to SEQ ID NO: 28 and the light chain comprises an amino acid sequence has at least 95% identity to SEQ ID NO: 77. In some embodiments, the heavy chain of the monoclonal antibody comprises an amino acid sequence has at least 95% identity to SEQ ID NO: 29 and the light chain comprises an amino acid sequence has at least 95% identity to SEQ ID NO: 80. In some embodiments, 95% identity to SEQ ID NO: 30 and the light chain comprises an amino acid sequence has at least 95% identity to SEQ ID NO: 81. In some embodiments, the heavy chain of the monoclonal antibody comprises an amino acid sequence has at least 95% identity to SEQ ID NO: 31 and the light chain comprises an amino acid sequence has at least 95% identity to SEQ ID NO: 82. In some embodiments, the heavy chain of the monoclonal antibody comprises an amino acid sequence has at least 95% identity to SEQ ID NO: 32 and the light chain comprises an amino acid sequence has at least 95% identity to SEQ ID NO: 84. In some embodiments, the heavy chain of the monoclonal antibody comprises an amino acid sequence has at least 95% identity to SEQ ID NO: 33 and the light chain comprises an amino acid sequence has at least 95% identity to SEQ ID NO: 85. In some embodiments, the heavy chain of the monoclonal antibody comprises an amino acid sequence has at least 95% identity to SEQ ID NO: 34 and the light chain comprises an amino acid sequence has at least 95% identity to SEQ ID NO: 86. In some embodiments, the heavy chain of the monoclonal antibody comprises an amino acid sequence has at least 95% identity to SEQ ID NO: 35 and the light chain comprises an amino acid sequence has at least 95% identity to SEQ ID NO: 87. In some embodiments, the heavy chain of the monoclonal antibody comprises an amino acid sequence has at least 95% identity to SEQ ID NO: 36 and the light chain comprises an amino acid sequence has at least 95% identity to SEQ ID NO: 88. In some embodiments, the heavy chain of the monoclonal antibody comprises an amino acid sequence has at least 95% identity to SEQ ID NO: 37 and the light chain comprises an amino acid sequence has at least 95% identity to SEQ ID NO: 90. In some embodiments, the heavy chain of the monoclonal antibody comprises an amino acid sequence has at least 95% identity to SEQ ID NO: 38 and the light chain comprises an amino acid sequence has at least 95% identity to SEQ ID NO: 91. In some embodiments, the heavy chain of the monoclonal antibody comprises an amino acid sequence has at least 95% identity to SEQ ID NO: 39 and the light chain comprises an amino acid sequence has at least 95% identity to SEQ ID NO: 92. In some embodiments, the heavy chain of the monoclonal antibody comprises an amino acid sequence has at least 95% identity to SEQ ID NO: 40 and the light chain comprises an amino acid sequence has at least 95% identity to SEQ ID NO: 93. In some embodiments, the heavy chain of the monoclonal antibody comprises an amino acid sequence has at least 95% identity to SEQ ID NO: 41 and the light chain comprises an amino acid sequence has at least 95% identity to SEQ ID NO: 94. In some embodiments, 95% identity to SEQ ID NO: 42 and the light chain comprises an amino acid sequence has at least 95% identity to SEQ ID NO: 95. In some embodiments, the heavy chain of the monoclonal antibody comprises an amino acid sequence has at least 95% identity to SEQ ID NO: 43 and the light chain comprises an amino acid sequence has at least 95% identity to SEQ ID NO: 96. In some embodiments, the heavy chain of the monoclonal antibody comprises an amino acid sequence has at least 95% identity to SEQ ID NO: 45 and the light chain comprises an amino acid sequence has at least 95% identity to SEQ ID NO: 97. In some embodiments, the heavy chain of the monoclonal antibody comprises an amino acid sequence has at least 95% identity to SEQ ID NO: 46 and the light chain comprises an amino acid sequence has at least 95% identity to SEQ ID NO: 98. In some embodiments, the heavy chain of the monoclonal antibody comprises an amino acid sequence has at least 95% identity to SEQ ID NO: 47 and the light chain comprises an amino acid sequence has at least 95% identity to SEQ ID NO: 99. In some embodiments, the heavy chain of the monoclonal antibody comprises an amino acid sequence has at least 95% identity to SEQ ID NO: 48 and the light chain comprises an amino acid sequence has at least 95% identity to SEQ ID NO: 100. In some embodiments, the heavy chain of the monoclonal antibody comprises an amino acid sequence has at least 95% identity to SEQ ID NO: 49 and the light chain comprises an amino acid sequence has at least 95% identity to SEQ ID NO: 101. In some embodiments, the heavy chain of the monoclonal antibody comprises an amino acid sequence has at least 95% identity to SEQ ID NO: 50 and the light chain comprises an amino acid sequence has at least 95% identity to SEQ ID NO: 102. In some embodiments, the heavy chain of the monoclonal antibody comprises an amino acid sequence has at least 95% identity to SEQ ID NO: 51 and the light chain comprises an amino acid sequence has at least 95% identity to SEQ ID NO: 103. In some embodiments, the heavy chain of the monoclonal antibody comprises an amino acid sequence has at least 95% identity to SEQ ID NO: 52 and the light chain comprises an amino acid sequence has at least 95% identity to SEQ ID NO: 104. In some embodiments, the heavy chain of the monoclonal antibody comprises an amino acid sequence has at least 95% identity to SEQ ID NO: 53 and the light chain comprises an amino acid sequence has at least 95% identity to SEQ ID NO: 105. In some embodiments, the heavy chain of the monoclonal antibody comprises an amino acid sequence has at least 95% identity to SEQ ID NO: 153 and the light chain comprises an amino acid sequence has at least 95% identity to SEQ ID NO: 106.

Another aspect provides a recombinant monoclonal antibody that specifically binds to herpes simplex virus glycoprotein B (gB) comprising a heavy chain comprising an amino acid sequence having at least 95% identity to an amino acid sequence listed in Table 3. In some embodiments, the heavy chain of the monoclonal antibody comprises an amino acid sequence has at least 95% identity to SEQ ID NO: 12-15 or 44.

Another aspect provides a recombinant monoclonal antibody that specifically binds to herpes simplex virus glycoprotein B (gB) comprising a light chain chain comprising an amino acid sequence having at least 95% identity to an amino acid sequence listed in Table 3. In some embodiments, the light chain comprises an amino acid sequence has at least 95% identity to SEQ ID NO: 78, 79, 83, or 89.

In another aspect, a recombinant monoclonal antibody is provided that specifically binds to herpes simplex virus glycoprotein D (gD) comprising a heavy chain comprising an amino acid sequence having at least 95% identity to an amino acid sequence listed in Table 3 and a light chain comprising an amino acid sequence having at least 95% identity to an amino acid sequence listed in Table 3. In some embodiments, the heavy chain of the monoclonal antibody comprises an amino acid sequence has at least 95% identity to SEQ ID NO: 107 and the light chain comprises an amino acid sequence has at least 95% identity to SEQ ID NO: 102. In some embodiments, the heavy chain of the monoclonal antibody comprises an amino acid sequence has at least 95% identity to SEQ ID NO: 108 and the light chain comprises an amino acid sequence has at least 95% identity to SEQ ID NO: 113. In some embodiments, the heavy chain of the monoclonal antibody comprises an amino acid sequence has at least 95% identity to SEQ ID NO: 109 and the light chain comprises an amino acid sequence has at least 95% identity to SEQ ID NO: 114. In some embodiments, the heavy chain of the monoclonal antibody comprises an amino acid sequence has at least 95% identity to SEQ ID NO: 110 and the light chain comprises an amino acid sequence has at least 95% identity to SEQ ID NO: 115. In some embodiments, the heavy chain of the monoclonal antibody comprises an amino acid sequence has at least 95% identity to SEQ ID NO: 111 and the light chain comprises an amino acid sequence has at least 95% identity to SEQ ID NO: 116.

Another aspect provides a pharmaceutical composition comprising a recombinant monoclonal antibody described herein and a pharmaceutically acceptable carrier.

Another aspect provides a method of treating a subject having or suspected of having a herpes simplex virus (HSV) infection, the method comprising administering to the subject a monoclonal antibody described herein or a pharmaceutical composition described herein.

Another aspect provides method of preventing a herpes simplex virus (HSV) infection in a subject exposed to or at risk of being exposed to HSV, the method comprising administering to the subject a monoclonal antibody described herein or a pharmaceutical composition described herein.

Another aspect provides a method of treating a herpes simplex virus (HSV) outbreak in a subject, the method comprising administering to the subject a monoclonal antibody described herein or a pharmaceutical composition described herein.

Another aspect provides a method of preventing a herpes simplex virus (HSV) outbreak in a subject having or suspected of having a latent HSV infection, the method comprising administering to the subject a monoclonal antibody described herein or a pharmaceutical composition described herein.

In some embodiments, the subject is administered with a therapeutically effective amount of a monoclonal antibody or a pharmaceutical composition of the present disclosure.

In some embodiments, the HSV infection is a latent infection. In some embodiments, the HSV infection is a primary infection. In some embodiments, the HSV infection is an HSV-1 or HSV-2 infection. In some embodiments, the monoclonal antibody is administered intravenously, intraperitoneally, intramuscularly, or subcutaneously. In some embodiments, the monoclonal antibody is administered intranasally, orally, vaginally, rectally, sublingually, or topically.

Another aspect provides a method of eliciting a cellular immune response and antibody-dependent cellular cytotoxicity against a herpes simplex virus (HSV) infected cell, the method comprising contacting the cell with a monoclonal antibody described herein or a pharmaceutical composition described herein. In some embodiments, the cell is a sensory neuron or an epithelial cell. In some embodiments, the cellular immune response activates an effector T cell. In some embodiments, the effector T cell is CD4+ or CD8+ T cell. In some embodiments, the cellular immune response inhibits a Treg cell.

In some embodiments, a monoclonal antibody described herein further comprises a detectable label.

Another aspect provides a nucleic acid molecule encoding a monoclonal antibody described herein. In some embodiments, the nucleic acid molecule is a cloning or expression vector. In some embodiments, a host cell comprises the nucleic acid molecule encoding the monoclonal antibody.

Another aspect provides a method of producing the monoclonal antibody of the present disclosure, wherein the method comprises the steps of: (i) culturing a host cell comprising a nucleic acid comprising a sequence encoding the monoclonal antibody of the present disclosure under conditions suitable to allow expression of said monoclonal antibody; and (ii) recovering the expressed monoclonal antibody.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A and FIG. 1B are graphs showing reactivity of donor plasma. FIG. 1A shows donor plasma reactivity to HSV glycoprotein B (gB). FIG. 1B shows donor plasma reactivity to HSV glycoprotein D (gD).

FIG. 2 is a schematic showing the gating strategy used to isolate memory B cells and antigen reactive B cells.

FIG. 3 is a schematic of the protocol used to generate and characterize recombinant antibodies.

FIG. 4A and FIG. 4B are graphs identifying HSV antigen-binding recombinant antibodies. FIG. 4A identifies recombinant antibodies that bind gD. FIG. 4B identifies recombinant antibodies that bind gB.

FIG. 5A and FIG. 5B are graphs of neutralization assays performed using the antibodies identified in FIGS. 4A and 4B. FIG. 4A shows the results of neutralization assays for antibodies that specifically bind gD. FIG. 4B shows the results of neutralization assays for antibodies that specifically bind gB.

FIG. 6 is a diagram of epitope binning using biolayer interferometry (BLI).

FIG. 7 is a table showing the results of BLI analysis of strong, moderate, and weak neutralizing anti-gB antibodies

FIG. 8 is a graph showing the fold induction of antibody-dependent cellular cytotoxicity induced by a subset of recombinant antibodies.

FIG. 9A and FIG. 9B are graphs showing fold induction of antibody-dependent cellular cytotoxicity induced by weak, moderate, and strong neutralizing antibodies. FIG. 9A shows the fold induction of antibody-dependent cellular cytotoxicity induced by weak, moderate, and strong neutralizing anti-gB antibodies. FIG. 9B shows the fold induction of antibody-dependent cellular cytotoxicity induced by weak, moderate, and strong neutralizing anti-gD antibodies.

### DETAILED DESCRIPTION

Described herein are isolated antibodies, particularly monoclonal antibodies, e.g., human monoclonal antibodies, or antigen binding fragments thereof, which specifically bind to an antigen of a herpes simplex virus (e.g., HSV-1 and/or HSV-2). As demonstrated herein, these antibodies unexpectedly have neutralizing capability and the can induce antibody-dependent cellular cytotoxicity (ADCC). This represents a significant advancement in monoclonal antibody-based treatments of HSV infection.

Accordingly, provided herein are isolated antibodies targeting antigens from HSV, methods of making such antibodies, nucleic acids or vectors encoding such antibodies, and pharmaceutical compositions formulated to contain such antibodies. Also provided herein are diagnostic, prognostic, and therapeutic methods of using such antibodies, and devices employing such antibodies and/or fragments.

### Definitions

In order that the present description may be more readily understood, certain terms are first defined. Additional definitions are set forth throughout the detailed description.

The term “antibody” as used to herein includes whole antibodies and any antigen binding fragments (i.e., “antigen-binding portions”) or single chains thereof. An “antibody” refers, in one embodiment, to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. In certain naturally occurring antibodies, the heavy chain constant region is comprised of three domains, CH1, CH2 and CH3. In certain naturally occurring antibodies, each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (KD) of 10⁻⁵ to 10⁻¹¹ M or less. Any KD greater than about 10⁻⁴ M is generally considered to indicate nonspecific binding. As used herein, an antibody that “binds specifically” to an antigen refers to an antibody that binds to the antigen and substantially identical antigens with high affinity, which means having a KD of 10⁻⁷ M or less, preferably 10⁻⁸ M or less, even more preferably 5×10⁻⁹ M or less, and most preferably between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, preferably at least 95%, more preferably at least 97%, or even more preferably at least 99% sequence identity to the sequence of the given antigen.

An immunoglobulin may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids. “Antibody” includes, by way of example, both naturally occurring and non-naturally occurring antibodies: monoclonal and polyclonal antibodies: chimeric and humanized antibodies: human and nonhuman antibodies: wholly synthetic antibodies; and single chain antibodies.

The term “antigen-binding portion” of an antibody, as used herein, refers to one or more fragments of an antibody that retain the ability to specifically bind to an antigen (e.g., an HSV antigen). Such “fragments” are, for example, between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding portion” of an antibody, include (i) a Fab fragment, a monovalent fragment consisting of the V_{L}, V_{H}, CL and CHI domains: (ii) a F(ab′)₂ fragment, a bivalent fragment comprising two Fab fragments linked by a disulfide bridge at the hinge region: (iii) a Fd fragment consisting of the V_{H} and CH1 domains: (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv): see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

“And/or” as used herein, for example, with option A and/or option B, encompasses the separate embodiments of (i) option A, (ii) option B, and (iii) option A plus option B.

A “CDR” of a variable domain are amino acid residues within the hypervariable region that are identified in accordance with the definitions of the Kabat, Chothia, the cumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., 1989, Nature 342:877-883. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., 1996, J. Mol. Biol., 262:732-745. In another approach, referred to herein as the “conformational definition” of CDRs. the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

The term “monoclonal antibody,” as used herein, refers to an antibody that displays a single binding specificity and affinity for a particular epitope or a composition of antibodies in which all antibodies display a single binding specificity and affinity for a particular epitope. Accordingly, the term “human monoclonal antibody” refers to an antibody or antibody composition that display(s) a single binding specificity and which has variable and optional constant regions derived from human germline immunoglobulin sequences. In one embodiment, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant human antibody” or “recombinant antibody” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23 (9): 1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The antibodies described herein may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized” antibody refers to an antibody in which some, most or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions, or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

“Allotype” refers to naturally occurring variants within a specific isotype group, which variants differ in a few amino acids (see, e.g., Jefferis et al. (2009) mAbs 1:1). Antibodies described herein may be of any allotype.

The phrases “an antibody recognizing an antigen” and “an antibody specific for an antigen” are used interchangeably herein with the term “an antibody that binds specifically to an antigen.”

An “isolated antibody,” as used herein, is intended to refer to an antibody which is substantially free of other antibodies having different antigenic specificities (e.g., an isolated antibody that specifically binds to a particular HSV antigen (e.g., an HSV gB or gD protein or fragment thereof is substantially free of antibodies that specifically bind antigens other than the particular HSV antigen).

An “effector function” refers to the interaction of an antibody Fc region with an Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and downregulation of a cell surface receptor (e.g., the B cell receptor: BCR). Such effector functions generally require the Fc region to be combined with a binding domain (e.g., an antibody variable domain).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice: FcγRIA, FcγRIIA, and FcγRIIIA in humans) and one inhibitory (FcγRIIB) receptor. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA, and IgD antibody isotypes, the Fc region comprises two identical protein fragments, derived from the second (C_{H2}) and third (C_{H3}) constant domains of the antibody's two heavy chains: IgM and IgE Fc regions comprise three heavy chain constant domains (C_{H} domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 (or amino acid between these two amino acids) to the carboxy-terminus of the heavy chain, wherein the numbering is according to the EU index as in Kabat. The C_{H2} domain of a human IgG Fc region extends from about amino acid 231 to about amino acid 340, whereas the C_{H3} domain is positioned on C-terminal side of a C_{H2} domain in an Fc region, i.e., it extends from about amino acid 341 to about amino acid 447 of an IgG. As used herein, the Fc region may be a native sequence Fc, including any allotypic variant, or a variant Fc (e.g., a non-naturally occurring Fc). Fc may also refer to this region in isolation or in the context of an Fc-comprising protein polypeptide such as a “binding protein comprising an Fc region,” also referred to as an “Fc fusion protein” (e.g., an antibody or immunoadhesin).

A “native sequence Fc region” or “native sequence Fc” comprises an amino acid sequence that is identical to the amino acid sequence of an Fc region found in nature. Native sequence human Fc regions include a native sequence human IgG1 Fc region: native sequence human IgG2 Fc region: native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1:1).

A “hinge,” “hinge domain,” “hinge region,” or “antibody hinge region” refers to the domain of a heavy chain constant region that joins the CH1 domain to the CH2 domain and includes the upper, middle, and lower portions of the hinge (Roux et al. J. Immunol. 1998 161:4083). The hinge provides varying levels of flexibility between the binding and effector regions of an antibody and also provides sites for intermolecular disulfide bonding between the two heavy chain constant regions. As used herein, a human hinge starts at Glu216 and ends at Gly237 for all IgG isotypes (Roux et al., 1998 J Immunol 161:4083).

The term “hinge” includes wildtype hinges as well as variants thereof (e.g., non-naturally-occurring hinges or modified hinges). For example, the term “IgG2 hinge” includes wildtype IgG2 hinge and variants having 1, 2, 3, 4, 5, 1-3, 1-5, 3-5 and/or at most 5, 4, 3, 2, or 1 mutations, e.g., substitutions, deletions or additions. Exemplary IgG2 hinge variants include IgG2 hinges in which 1, 2, 3 or all 4 cysteines (C219, C220, C226 and C229) are changed to another amino acid. In a specific embodiment, an IgG2 comprises a C219S substitution. In certain embodiments, a hinge is a hybrid hinge that comprises sequences from at least two isotypes. For example, a hinge may comprise the upper, middle or lower hinge from one isotype and the remainder of the hinge from one or more other isotypes. For example, a hinge can be an IgG2/IgG1 hinge, and may comprise, e.g., the upper and middle hinges of IgG2 and the lower hinge of IgG1. A hinge may have effector function or be deprived of effector function. For example, the lower hinge of wildtype IgG1 provides effector function.

The term “CH1 domain” refers to the heavy chain constant region linking the variable domain to the hinge in a heavy chain constant domain. As used herein, a CH1 domain starts at A118 and ends at V215. The term “CH1 domain” includes wildtype CH1 domains as well as variants thereof (e.g., non-naturally-occurring CH1 domains or modified CH1 domains). For example, the term “CH1 domain” includes wildtype CH1 domains and variants having 1, 2, 3, 4, 5, 1-3, 1-5, 3-5 and/or at most 5, 4, 3, 2, or 1 mutations, e.g., substitutions, deletions or additions. Exemplary CH1 domains include CH1 domains with mutations that modify a biological activity of an antibody, such as ADCC, CDC or half-life. Modifications to the CH1 domain that affect a biological activity of an antibody are provided herein.

The term “CH2 domain” refers to the heavy chain constant region linking the hinge to the CH3 domain in a heavy chain constant domain. As used herein, a CH2 domain starts at P238 and ends at K340. The term “CH2 domain” includes wildtype CH2 domains, as well as variants thereof (e.g., non-naturally-occurring CH2 domains or modified CH2 domains). For example, the term “CH2 domain” includes wildtype CH2 domains and variants having 1, 2, 3, 4, 5, 1-3, 1-5, 3-5 and/or at most 5, 4, 3, 2, or 1 mutations, e.g., substitutions, deletions or additions. Exemplary CH2 domains include CH2 domains with mutations that modify a biological activity of an antibody, such as ADCC, CDC or half-life. In certain embodiments, a CH2 domain comprises the substitutions A330S/P331S that reduce effector function. Other modifications to the CH2 domain that affect a biological activity of an antibody are provided herein.

The term “CH3 domain” refers to the heavy chain constant region that is C-terminal to the CH2 domain in a heavy chain constant domain. As used herein, a CH3 domain starts at G341 and ends at K447. The term “CH3 domain” includes wildtype CH3 domains, as well as variants thereof (e.g., non-naturally-occurring CH3 domains or modified CH3 domains). For example, the term “CH3 domain” includes wildtype CH3 domains and variants having 1, 2, 3, 4, 5, 1-3, 1-5, 3-5 and/or at most 5, 4, 3, 2, or 1 mutations, e.g., substitutions, deletions or additions. Exemplary CH3 domains include CH3 domains with mutations that modify a biological activity of an antibody, such as ADCC, CDC or half-life. Modifications to the CH3 domain that affect a biological activity of an antibody are provided herein.

A “native sequence Fc region” or “native sequence Fc” comprises an amino acid sequence that is identical to the amino acid sequence of an Fc region found in nature. Native sequence human Fc regions include a native sequence human IgG1 Fc region: native sequence human IgG2 Fc region: native sequence human IgG3 Fc region; and native sequence human IgG4 Fc region as well as naturally occurring variants thereof. Native sequence Fc includes the various allotypes of Fcs (see, e.g., Jefferis et al. (2009) mAbs 1:1).

The term “epitope” or “antigenic determinant” refers to a site on an antigen (e.g., an HSV antigen) to which an immunoglobulin or antibody specifically binds. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from an HSV antigen (e.g., a gB or gD protein) are tested for reactivity with a given antibody (e.g., anti-HSV gB or gD protein antibody). Methods of determining spatial conformation of epitopes include techniques in the art and those described herein, for example, x-ray crystallography, 2-dimensional nuclear magnetic resonance and HDX-MS (see, e.g., Epitope Mapping Protocols in Methods in Molecular Biology. Vol. 66, G. E. Morris, Ed. (1996)).

The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the same epitope with the antibodies described herein include, for example, epitope mapping methods, such as, x-ray analyses of crystals of antigen: antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, may be determined using known competition experiments. In certain embodiments, an antibody competes with, and inhibits binding of another antibody to a target by at least 10%, 20%, 30%, 40%, 50%, 60%, 70%, 80%, 90%, or 100%. The level of inhibition or competition may be different depending on which antibody is the “blocking antibody” (i.e., the cold antibody that is incubated first with the target). Competition assays can be conducted as described, for example, in Ed Harlow and David Lane, Cold Spring Harb Protoc: 2006; doi: 10.1101/pdb.prot4277 or in Chapter 11 of “Using Antibodies” by Ed Harlow and David Lane, Cold Spring Harbor Laboratory Press, Cold Spring Harbor, NY, USA 1999. Competing antibodies bind to the same epitope, an overlapping epitope or to adjacent epitopes (e.g., as evidenced by steric hindrance).

Other competitive binding assays include: biolayer interferometry, solid phase direct or indirect radioimmunoassay (RIA), solid phase direct or indirect enzyme immunoassay (EIA), sandwich competition assay (see Stahli et al., Methods in Enzymology 9:242 (1983)): solid phase direct biotin-avidin EIA (see Kirkland et al., J. Immunol. 137:3614 (1986)): solid phase direct labeled assay, solid phase direct labeled sandwich assay (see Harlow and Lane, Antibodies: A Laboratory Manual, Cold Spring Harbor Press (1988)): solid phase direct label RIA using I-125 label (see Morel et al., Mol. Immunol. 25 (1): 7 (1988)): solid phase direct biotin-avidin EIA (Cheung et al., Virology 176:546 (1990)); and direct labeled RIA. (Moldenhauer et al., Scand. J. Immunol. 32:77 (1990)).

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE 2000 instrument using the predetermined antigen, e.g., recombinant SARS-CoV-2 antigen, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antibody that “specifically binds to an HSV antigen” refers to an antibody that binds to a soluble or cell bound HSV antigen with a Kp of 10⁻⁷ M or less, such as approximately less than 10⁻⁸ M, 10⁻⁹ M or 10⁻¹⁰ M or even lower.

The term “k_{assoc}” or “kₐ,” as used herein, is intended to refer to the association rate of a particular antibody-antigen interaction, whereas the term “k_{dis}” or “k_{d},” as used herein, is intended to refer to the dissociation rate of a particular antibody-antigen interaction. The term “K_{D},” as used herein, is intended to refer to the dissociation constant, which is obtained from the ratio of k_{d} to kₐ (i.e,. k_{d}/kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore R system or flow cytometry and Scatchard analysis.

As used herein, the term “high affinity” for an IgG antibody refers to an antibody having a K_{D} of 10⁻⁸ M or less, more preferably 10⁻⁹ M or less and even more preferably 10⁻¹⁰ M or less for a target antigen. However, “high affinity” binding can vary for other antibody isotypes. For example, “high affinity” binding for an IgM isotype refers to an antibody having a K_{D} of 10⁻⁷ M or less, more preferably 10⁻⁸ M or less.

The term “EC50” in the context of an in vitro or in vivo assay using an antibody or antigen binding fragment thereof, refers to the concentration of an antibody or an antigen-binding portion thereof that induces a response that is 50% of the maximal response, i.e., halfway between the maximal response and the baseline.

The term “binds to an immobilized HSV antigen,” refers to the ability of an antibody described herein to bind to an HSV antigen, for example, expressed on the surface of a cell or which is attached to a solid support.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein may contain a modification such as, but not limited to, glycosylation, phosphorylation or disulfide bond formation. A “protein” may comprise one or more polypeptides.

The term “nucleic acid molecule,” as used herein, is intended to include DNA molecules and RNA molecules. A nucleic acid molecule may be single-stranded or double-stranded, and may be cDNA.

The nucleic acid compositions of the present invention, while often in a native sequence (except for modified restriction sites and the like), from either cDNA, genomic or mixtures thereof may be mutated, in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, cDNA, or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

Also contemplated are “conservative sequence modifications” of the sequences of, or encoding, the antibodies described herein, i.e., nucleotide and amino acid sequence modifications which do not abrogate the binding of the antibody encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into a sequence by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an antibody is preferably replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993): Kobayashi et al. Protein Eng. 12 (10): 879-884 (1999); and Burks et al. Proc. Natl. Acad. Sci. USA 94:412-417 (1997)). Alternatively, in another embodiment, mutations can be introduced randomly along all or part of an antibody coding sequence, such as by saturation mutagenesis, and the resulting modified antibodies can be screened for binding activity.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, usually at least about 90% to 95%, and more preferably at least about 98% to 99.5% of the amino acids.

As used herein, the term “percent sequence identity” refers to the degree of identity between any given query sequence and a subject sequence. A percent identity for any query nucleic acid or amino acid sequence, e.g., a transcription factor, relative to another subject nucleic acid or amino acid sequence can be determined as follows. The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region. It is noted that the percent identity value is usually rounded to the nearest integer. For example, 78.1%, 78.2%, 78.3%, and 78.4% are rounded down to 78%, while 78.5%, 78.6%, 78.7%, 78.8%, and 78.9% are rounded up to 79%. It is also noted that the length of the aligned region is always an integer.

The percent identity between two nucleotide sequences can be determined using the GAP program in the GCG software package (available on the World Wide Web at gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48): 444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used. See World Wide Web at ncbi.nlm.nih.gov.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, may be mutated, in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. For example, “vector”, “cloning vector,” and “expression vector” mean the vehicle by which a DNA or RNA sequence (e.g. a foreign gene) can be introduced into a host cell, so as to transform the host and promote expression (e.g. transcription and translation) of the introduced sequence. Thus, a further object of the invention relates to a vector comprising a nucleic acid of the present invention.

One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and maybe a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

As used herein, the term “antigen” refers to any natural or synthetic immunogenic substance, such as a protein, peptide, or hapten. An antigen may be an HSV antigen (e.g., an HSV gB or gD protein), or a fragment thereof. Representative sequences of HSV gB and gD proteins are provided in Table 1.

TABLE 1
Representative Human Herpes Simplex Virus glycoprotein B and D
sequences

>AF097023.1 Human herpesvirus 1 strain HSZP glycoprotein B (UL27) gene,
complete cds
ATGCGCCAGGGCGCCCCCGCGCGGGGGTGCCGGTGGTTCGTCGTATGGGCGCTCTTGGGGTTGACGCTGG
GGGTCCTGGTGGCGTCGGCGGCTCCGAGTTCCCCCGGCACGCCTGGGGTCGCGGCCGCGACCCAGGCGGC
GAACGGGGGCCCTGCCACTCCGGCGCCGCCCGCCCTTGGCGCCGCCCCAACGGGGGACCCGAAACCGAAG
AAGAACAAAAAACCGAAAAACCCAACGCCGCCACGCCCCGCCGGCGACAACGCGACCGTCGCCGCGGGCC
ACGCCACCCTGCGCGAGCACCTGCGGGACATCAAGGCGGAGAGCACCGATGCAAACTTTTACGTGTGCCC
ACCCCCCACGGGCGCCACGGTGGTGCAGTTCGAGCAGCCGCGCCGCTGCCCGACCCGGCCCGAGGGTCAG
AACTACACGGAGGGCATCGCGGTGGTCTTCAAGGAGAACATCGCCCCGTACAAGTTCAAGGCCACCATGT
ACTACAAAGACGTCACCGTTTCGCAGGTGTGGTTCGGCCACCGCTACTCCCAGTTTATGGGGATCTTTGA
GGACCGCGCCCCCGTCCCCTTCGAGGAGGTGATCGACAAGATCAACGCCAAGGGGGTCTGTCGGTCCACG
GCCAAGTACGTGCGCAACAACCTGGAGACCACCGCGTTTCACCGGGACGACCACGAGACCGACATGGAGC
TGAAACCGGCCAATGCCGCGACCCGCACGAGCCGGGGCTGGCACACCACCGACCTCAAGTACAACCCCTC
GCGGGTGGAGGCGTTCCACCGGTACGGGACGACGGTAAACTGCATCGTCGAGGAGGTGGACGCGCGCTCG
GTGTACCCGTACGACGAGTTTGTGCTGGCGACTGGCGACTTTGTGTACATGTCCCCGTTTTACGGCTACC
GGGAGGGGTCGCACACCGAACACACCAGCTACGCCGCCGACCGCTTCAAGCAGGTCGACGGCTTCTACGC
GCGCGACCTCACCACCAAGGCCCGGGCCACGGCGCCGACCACCCGGAACCTGCTCACGACCCCCAAGTTC
ACCGTGGCCTGGGACTGGGTGCCAAAGCGCCCGTCGGTCTGCACCATGACCAAGTGGCAGGAGGTGGACG
AGATGCTGCGCTCCGAGTACGGCGGCTCCTTCCGATTCTCCTCCGACGCCATATCCACCACCTTCACCAC
CAACCTGACCGAGTACCCGCTCTCGCGCGTGGACCTGGGGGACTGCATCGGCAAGGACGCCCGCGACGCC
ATGGACCGCATCTTCGCCCGCAGGTACAACGCGACGCACATTAAGGTGGGCCAGCCGCAGTACTACCTGG
CCAATGGGGGCTTTCTGATCGCGTACCAGCCCCTTCTCAGCAACACGCTCGCGGAGCTGTACGTGCGGGA
ACACCTCCGAGAGCAGAGCCGCAAGCCCCCAAACCCCACGCCCCCGCCGCCCGGGGCCAGCGCCAACGCG
TCCGTGGAGCGCATCAAGACCACCTCCTCCATCGAGTTCGCCCGGCTGCAGTTTACGTACAACCACATAC
AGCGCCATGTCAACGATATGTTGGGCCGCGTTGCCATCGCGTGGTGCGAGCTGCAGAATCACGAGCTGAC
CCTGTGGAACGAGGCCCGCAAGCTGAACCCCAACGCCATTGCATCGGCCACCGTGGGCCGGCGGGTGAGC
GCGCGGATGCTCGGCGACGTGATGGCCGTCTCCACGTGCGTGCCGGTCGCCGCGGACAACGTGATCGTCC
AAAACTCGATGCGCATCAGCTCGCGGCCCGGGGCCTGCTACAGCCGCCCCCTGGTCAGCTTTCGGTACGA
AGACCAGGGCCCGTTGGTCGAGGGGCAGCTGGGGGAGAACAACGAGCTGCGGCTGACGCGCGATGCGATC
GAGCCGTGCACCGTGGGACACCGGCGCTACTTCACCTTCGGTGGGGGCTACGTGTACTTCGAGGAGTACG
CGTACTCCCACCAGCTGAGCCGCGCCGACATCACCACCGTCAGCACCTTCATCGACCTCAACATCACCAT
GCTGGAGGATCACGAGTTTGTCCCCCTGGAGGTGTACACCCGCCACGAGATCAAGGACAGCGGCCTGCTG
GACTACACGGAGGTCCAGCGCCGCAACCAGCTGCACGACCTGCGCTTTGCCGACATCGACACGGTCATCC
ACGCCGACGCCAACGCCGCCATGTTCGCGGGCCTGGGTGCGTTTTTCGAGGGGATGGGCGACCTGGGGCG
CGCGGTCGGCAAGGTGGTGATGGGCATCGTGGGCGGCGTGGTATCGGCCGTGTCGGGCGTGTCCTCCTTC
ATGTCCAACCCCTTTGGGGCGCTGGCCGTGGGTCTGTTGGTCCTGGCCGGCCTGGCGGCGGCCTTCTTCG
CCTTTCGCTACGTCATGCGGCTGCAGAGCAACCCCATGAAGGCCCTGTACCCGCTAACCACCAAGGAGCT
CAAGAACCCCACCAACCCGGACGCGTCCGGGGAGGGCGAGGAGGGCGGCGACTTTGACGAGGCCAAGCTA
GCCGAGGCCCGGGAGATGATACGGTACATGGCCCTGGTGTCGGCCATGGAGCACACGGAACACAAGGCCA
AGAAGAAGGGCACGAGCGCGCTGCTTAGCGCCAAGGTCACCGACATGGTCATGCGCAAGCGCCGCAACAC
CAACTACACCCAAGTTCCCAACAAAGACGGTGACGCCGACGAGGACGACCTGTGA
SEQ ID NO: 201)
>AAF04615.1 glycoprotein B [Human alphaherpesvirus 1]
MRQGAPARGCRWFVVWALLGLTLGVLVASAAPSSPGTPGVAAATQAANGGPATPAPPALGAAPTGDPKPK
KNKKPKNPTPPRPAGDNATVAAGHATLREHLRDIKAESTDANFYVCPPPTGATVVQFEQPRRCPTRPEGQ
NYTEGIAVVFKENIAPYKFKATMYYKDVTVSQVWFGHRYSQFMGIFEDRAPVPFEEVIDKINAKGVCRST
AKYVRNNLETTAFHRDDHETDMELKPANAATRTSRGWHTTDLKYNPSRVEAFHRYGTTVNCIVEEVDARS
VYPYDEFVLATGDFVYMSPFYGYREGSHTEHTSYAADRFKQVDGFYARDLTTKARATAPTTRNLLTTPKF
TVAWDWVPKRPSVCTMTKWQEVDEMLRSEYGGSFRFSSDAISTTFTTNLTEYPLSRVDLGDCIGKDARDA
MDRIFARRYNATHIKVGQPQYYLANGGFLIAYQPLLSNTLAELYVREHLREQSRKPPNPTPPPPGASANA
SVERIKTTSSIEFARLQFTYNHIQRHVNDMLGRVAIAWCELQNHELTLWNEARKLNPNAIASATVGRRVS
ARMLGDVMAVSTCVPVAADNVIVQNSMRISSRPGACYSRPLVSFRYEDQGPLVEGQLGENNELRLTRDAI
EPCTVGHRRYFTFGGGYVYFEEYAYSHQLSRADITTVSTFIDLNITMLEDHEFVPLEVYTRHEIKDSGLL
DYTEVQRRNQLHDLRFADIDTVIHADANAAMFAGLGAFFEGMGDLGRAVGKVVMGIVGGVVSAVSGVSSF
MSNPFGALAVGLLVLAGLAAAFFAFRYVMRLQSNPMKALYPLTTKELKNPTNPDASGEGEEGGDEDEAKL
AEAREMIRYMALVSAMEHTEHKAKKKGTSALLSAKVTDMVMRKRRNTNYTQVPNKDGDADEDDL
(SEQ ID NO: 202)
>JQ320083.1 Human herpesvirus 1 strain McKrae glycoprotein D (gD) gene,
complete cds
ATGGGGGGGGCTGCCGCCAGGTTGGGGGCCGTGATTTTGTTTGTCGTCATAGTGGGCCTCCATGGGGTCC
GCGGCAAATATGCCTTGGCGGATGCCTCTCTCAAGATGGCCGACCCCAATCGCTTTCGCGGCAAAGACCT
TCCGGTCCTGGACCAGCTGACCGACCCTCCGGGGGTCCGGCGCGTGTACCACATCCAGGCGGGCCTACCG
GACCCGTTCCAGCCCCCCAGCCTCCCGATCACGGTTTACTACGCCGTGTTGGAGCGCGCCTGCCGCAGCG
TGCTCCTAAACGCACCGTCGGAGGCCCCCCAGATTGTCCGCGGGGCCTCCGAAGACGTCCGGAAACAACC
CTACAACCTGACCATCGCTTGGTTTCGGATGGGAGGCAACTGTGCTATCCCCATCACGGTCATGGAGTAC
ACCGAATGCTCCTACAACAAGTCTCTGGGGGCCTGTCCCATCCGAACGCAGCCCCGCTGGAACTACTATG
ACAGCTTCAGCGCCGTCAGCGAGGATAACCTGGGGTTCCTGATGCACGCCCCCGCGTTTGAGACCGCCGG
CACGTACCTGCGGCTCGTGAAGATAAACGACTGGACGGAGATTACACAGTTTATCCTGGAGCACCGAGCC
AAGGGCTCCTGTAAGTACGCCCTCCCGCTGCGCATCCCCCCGTCAGCCTGCCTGTCCCCCCAGGCCTACC
AGCAGGGGGTGACGGTGGACAGCATCGGGATGCTGCCCCGCTTCATCCCCGAGAACCAGCGCACCGTCGC
CGTATACAGCTTGAAGATCGCCGGGTGGCACGGGCCCAAGGCCCCATACACGAGCACCCTGCTGCCCCCG
GAGCTGTCCGAGACCCCCAACGCCACGCAGCCAGAACTCGCCCCGGAAGACCCCGAGGATTCGGCCCTCT
TGGAGGACCCCGTGGGGACGGTGGCGCCGCAAATCCCACCAAACTGGCACATACCGTCGATCCAGGACGC
CGCGACGCCTTACCATCCCCCGGCCACCCCGAACAACATGGGCCTGATCGCCGGCGCGGTGGGCGGCAGT
CTCCTGGCAGCCCTGGTCATTTGCGGAATTGTGTACTGGATGCGCCGCCGCACTCAAAAAGCCCCAAAGC
GCATACGCCTCCCCCACATCCGGGAAGACGACCAGCCGTCCTCGCACCAGCCCTTGTTTTACTAG
(SEQ ID NO: 203)
>AFH41180.1 glycoprotein D [Human alphaherpesvirus 1]
MGGAAARLGAVILFVVIVGLHGVRGKYALADASLKMADPNRFRGKDLPVLDQLTDPPGVRRVYHIQAGLP
DPFQPPSLPITVYYAVLERACRSVLLNAPSEAPQIVRGASEDVRKQPYNLTIAWERMGGNCAIPITVMEY
TECSYNKSLGACPIRTQPRWNYYDSFSAVSEDNLGFLMHAPAFETAGTYLRLVKINDWTEITQFILEHRA
KGSCKYALPLRIPPSACLSPQAYQQGVTVDSIGMLPRFIPENQRTVAVYSLKIAGWHGPKAPYTSTLLPP
ELSETPNATQPELAPEDPEDSALLEDPVGTVAPQIPPNWHIPSIQDAATPYHPPATPNNMGLIAGAVGGS
LLAALVICGIVYWMRRRTQKAPKRIRLPHIREDDQPSSHQPLFY
(SEQ ID NO: 204)

An “immune response” refers to a biological response within a vertebrate against foreign agents, which response protects the organism against these agents and diseases caused by them. An immune response is mediated by the action of a cell of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4+ or CD8+ T cell, or the inhibition of a Treg cell.

An “immunomodulator” or “immunoregulator” refers to an agent, e.g., a component of a signaling pathway that may be involved in modulating, regulating, or modifying an immune response. “Modulating,” “regulating,” or “modifying” an immune response refers to any alteration in a cell of the immune system or in the activity of such cell (e.g., an effector T cell). Such modulation includes stimulation or suppression of the immune system which may be manifested by an increase or decrease in the number of various cell types, an increase or decrease in the activity of these cells, or any other changes which can occur within the immune system. Both inhibitory and stimulatory immunomodulators have been identified. In preferred embodiments, the immunomodulator is located on the surface of a T cell. An “immunomodulatory target” or “immunoregulatory target” is an immunomodulator that is targeted for binding by, and whose activity is altered by the binding of, a substance, agent, moiety, compound or molecule. Immunomodulatory targets include, for example, receptors on the surface of a cell (“immunomodulatory receptors”) and receptor ligands (“immunomodulatory ligands”).

“Immunotherapy” refers to the treatment of a subject afflicted with, or at risk of contracting or suffering a recurrence of, a disease by a method comprising inducing, enhancing, suppressing or otherwise modifying an immune response.

“Immunostimulating therapy” or “immunostimulatory therapy” refers to a therapy that results in increasing (inducing or enhancing) an immune response in a subject for, e.g., treating viral infection.

“Potentiating an endogenous immune response” means increasing the effectiveness or potency of an existing immune response in a subject. This increase in effectiveness and potency may be achieved, for example, by overcoming mechanisms that suppress the endogenous host immune response or by stimulating mechanisms that enhance the endogenous host immune response.

“T effector” (“Teff”) cells refers to T cells (e.g., CD4+ and CD8+ T cells) with cytolytic activities as well as T helper (Th) cells, which secrete cytokines and activate and direct other immune cells, but does not include regulatory T cells (Treg cells).

An increased ability to stimulate an immune response or the immune system, can result from an enhanced agonist activity of T cell costimulatory receptors and/or an enhanced antagonist activity of inhibitory receptors. An increased abililty to stimulate an immune response or the immune system may be reflected by a fold increase of the EC₅₀ or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity may be enhanced by at least 10%, 30%, 50%, 75%, 2 fold, 3 fold, 5 fold, or more.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, “administering” refers to the physical introduction of a composition comprising a therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually, or topically. Administering can also be performed, for example, once, a plurality of times, and/or over one or more extended periods.

As used herein, the term “T cell-mediated response” refers to a response mediated by T cells, including effector T cells (e.g., CD8⁺ cells) and helper T cells (e.g., CD4⁺ cells). T cell mediated responses include, for example, T cell cytotoxicity and proliferation.

As used herein, the term “cytotoxic T lymphocyte (CTL) response” refers to an immune response induced by cytotoxic T cells. CTL responses are mediated primarily by CD8⁺ T cells.

As used herein, the terms “inhibits” or “blocks” (e.g., referring to inhibition/blocking of an HSV infection or binding of an HSV glycoprotein (e.g., gB or gD) protein to a host cell surface protein or other molecule on the host cell surface) are used interchangeably and encompass both partial and complete inhibition/blocking. In some embodiments, the antibodies described herein inhibit HSV infection, or binding of an HSV glycoprotein to a host cell surface protein or other molecule on the host cell surface by at least about 50%, for example, about 60%, 70%, 80%, 90%, 95%, 99%, or 100%, determined, e.g., as further described herein. In some embodiments the antibodies described herein inhibit HSV infection or binding of an HSV glycoprotein to a cell surface protein or other molecule on the host cell surface by no more than 50%, for example, by about 40%, 30%, 20%, 10%, 5% or 1%, determined, e.g., as further described herein.

As used herein, the term “reduce activity” of an HSV antigen (e.g., HSV gB or gD protein) includes any measurable decrease in the activity of the HSV antigen, e.g., the inhibition of the activity of an HSV antigen by at least about 10%, for example, at least about 20%, at least about 30%, at least about 40%, at least about 50%, at least about 60%, at least about 70%, at least about 80%, at least about 90%, at least about 99%, or 100%.

As used herein, “latency” or “latent infection” refers to an existing yet quiescent HSV infection. HSV can establish latency in certain sensory neurons of infected individuals. Outbreaks occur when a latent transcriptional program switches to a viral lytic transcriptional program. Outbreaks can result from environmental stressors as well as an impaired immune system, stress, and other infections. An outbreak in an individual often results in characteristic herpetic lesions (i.e., genital or oral blisters). Generally during a latent infection, disease is absent and no infectious virus is produced.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, or a prevention of impairment or disability due to the disease affliction. A therapeutically effective amount or dosage of a drug includes a “prophylactically effective amount” or a “prophylactically effective dosage,” which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

The term “patient” includes human and other mammalian subjects that receive either prophylactic or therapeutic treatment.

As used herein, the term “subject” includes any human or non-human animal. For example, the methods and compositions described herein can be used to treat a subject having an HSV infection (e.g., active or latent infection). The term “non-human animal” includes all vertebrates, e.g., mammals and non-mammals, such as non-human primates, sheep, dog, cow, chickens, amphibians, reptiles, etc.

As used herein, the terms “ug” and “uM” are used interchangeably with “ug” and “UM”.

The articles “a” and “an” are used herein to refer to one or to more than one (i.e., to at least one) of the grammatical object of the article. By way of example, “an element” means one element or more than one element.

The amount of a biomarker (e.g., an HSV antigen) in a subject is “significantly” higher or lower than the normal amount of the biomarker, if the amount of the biomarker is greater or less, respectively, than the normal or control level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternatively, the amount of the biomarker in the subject can be considered “significantly” higher or lower than the normal and/or control amount if the amount is at least about two, and preferably at least about 5%, 10%, 15%, 20%, 25%, 30%, 35%, 40%, 45%, 50%, 55%, 60%, 65%, 70%, 75%, 80%, 85%, 90%, 95%, 100%, 105%, 110%, 115%, 120%, 125%, 130%, 135%, 140%, 145%, 150%, 155%, 160%, 165%, 170%, 175%, 180%, 185%, 190%, 195%, two times, three times, four times, five times, or more, or any range in between, such as 5%-100%, higher or lower, respectively, than the normal and/or control amount of the biomarker. Such significant modulation values can be applied to any metric described herein, such as altered level, altered activity, changes in biomarker inhibition/blocking, changes in test agent binding, and the like.

The “amount” of a marker, e.g., level of an HSV antigen, in a subject is “significantly” higher or lower than the normal amount of a marker, if the amount of the marker is greater or less, respectively, than the normal level by an amount greater than the standard error of the assay employed to assess amount, and preferably at least twice, and more preferably three, four, five, ten or more times that amount. Alternately, the amount of the marker in the subject can be considered “significantly” higher or lower than the normal amount if the amount is at least about two, and preferably at least about three, four, or five times, higher or lower, respectively, than the normal amount of the marker.

The term “control” refers to any reference standard suitable to provide a comparison to the antigens (e.g., HSV viral antigens) in the test sample. In one embodiment, the control comprises obtaining a “control sample” from which antigen levels are detected and compared to the antigen levels from the test sample. Such a control sample may comprise any suitable sample, including but not limited to a sample from a control HSV patient (can be stored sample or previous sample measurement) with a known outcome: normal tissue or cells isolated from a subject, such as a normal patient or the HSV patient, cultured primary cells/tissues isolated from a subject such as a normal subject or the HSV patient, adjacent normal cells/tissues obtained from the same organ or body location of the HSV patient, a tissue or cell sample isolated from a normal subject, or a primary cells/tissues obtained from a depository. In another preferred embodiment, the control may comprise a reference standard antigen level from any suitable source, including but not limited to housekeeping genes, an antigen level range from normal tissue (or other previously analyzed control sample), a previously determined antigen level range within a test sample from a group of patients, or a set of patients with a certain outcome or receiving a certain treatment. It will be understood by those of skill in the art that such control samples and reference standard antigen levels can be used in combination as controls in the methods of the present invention.

The “normal” level of a marker is the level of the marker in cells of a subject, e.g., a human patient, not afflicted with a disease or disorder related to aberrant marker levels.

Such antibodies, described herein, can be used in any one of well-known immunoassay forms, including, without limitation, a radioimmunoassay, a Western blot assay, an immunofluorescence assay, an enzyme immunoassay, an immunoprecipitation assay, a chemiluminescence assay, an immunohistochemical assay, a dot blot assay, or a slot blot assay. General techniques to be used in performing the various immunoassays noted above and other variations of the techniques, such as in situ proximity ligation assay (PLA), fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA), ELISA, etc. alone or in combination or alternatively with NMR, MALDI-TOF, LC-MS/MS, are known to those of ordinary skill in the art.

Such reagents can also be used to monitor protein levels in a cell or tissue, e.g., white blood cells or lymphocytes, as part of a clinical testing procedure, e.g., in order to monitor an optimal dosage of an inhibitory agent. Detection can be facilitated by coupling (e.g., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase: examples of suitable prosthetic group complexes include streptavidin/biotin and avidin/biotin: examples of suitable fluorescent materials include umbelliferone, fluorescein, fluorescein isothiocyanate, rhodamine, dichlorotriazinylamine fluorescein, dansyl chloride or phycoerythrin: an example of a luminescent material includes luminol: examples of bioluminescent materials include luciferase, luciferin, and aequorin, and examples of suitable radioactive material include ¹²⁵I, ¹³¹I, ³⁵S or ³H.

Such reagents can also be used with any number of biological samples. Biological samples can be collected from a variety of sources from a patient including a body fluid sample, cell sample, or a tissue sample comprising nucleic acids and/or proteins. In a preferred embodiment, the subject and/or control sample is selected from the group consisting of cells, cell lines, histological slides, paraffin embedded tissues, biopsies, whole blood, nipple aspirate, serum, plasma, buccal scrape, saliva, cerebrospinal fluid, urine, stool, and bone marrow. In one embodiment, the sample is serum, plasma, or urine. In another embodiment, the sample is serum.

The samples can be collected from individuals repeatedly over a longitudinal period of time (e.g., once or more on the order of days, weeks, months, annually, biannually, etc.). Obtaining numerous samples from an individual over a period of time can be used to verify results from earlier detections and/or to identify an alteration in biological pattern as a result of, for example, disease progression, drug treatment, etc. For example, subject samples can be taken and monitored every month, every two months, or combinations of one, two, or three month intervals according to the present invention. In addition, the biomarker amount and/or activity measurements of the subject obtained over time can be conveniently compared with each other, as well as with those of normal controls during the monitoring period, thereby providing the subject's own values, as an internal, or personal, control for long-term monitoring.

Samples can contain live cells/tissue, fresh frozen cells, fresh tissue, biopsies, fixed cells/tissue, cells/tissue embedded in a medium, such as paraffin, histological slides, or any combination thereof.

Sample preparation and separation can involve any of the procedures, depending on the type of sample collected and/or analysis of biomarker measurement(s). Such procedures include, by way of example only, concentration, dilution, adjustment of pH, removal of high abundance polypeptides (e.g., albumin, gamma globulin, and transferrin, etc.), addition of preservatives and calibrants, addition of protease inhibitors, addition of denaturants, desalting of samples, concentration of sample proteins, extraction and purification of lipids.

The sample preparation can also isolate molecules that are bound in non-covalent complexes to other protein (e.g., carrier proteins). This process may isolate those molecules bound to a specific carrier protein (e.g., albumin), or use a more general process, such as the release of bound molecules from all carrier proteins via protein denaturation, for example using an acid, followed by removal of the carrier proteins.

Removal of undesired proteins (e.g., high abundance, uninformative, or undetectable proteins) from a sample can be achieved using high affinity reagents, high molecular weight filters, ultracentrifugation and/or electrodialysis. High affinity reagents include antibodies or other reagents (e.g., aptamers) that selectively bind to high abundance proteins. Sample preparation could also include ion exchange chromatography, metal ion affinity chromatography, gel filtration, hydrophobic chromatography, chromatofocusing, adsorption chromatography, isoelectric focusing and related techniques. Molecular weight filters include membranes that separate molecules on the basis of size and molecular weight. Such filters may further employ reverse osmosis, nanofiltration, ultrafiltration and microfiltration.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of an HSV infection or outbreak or the likelihood of recovery from an HSV infection or outbreak. In some embodiments, the use of statistical algorithms provides a prognosis of an HSV infection or outbreak in an individual. A prognosis can inform a practitioner the extent of an active infection and/or the probability of a latent infection to become an active one.

The terms “response” or “responsiveness” refers to response to a therapy. For example, an anti-viral response includes reduction of viral load or inhibiting viral infection. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a subject will exhibit a favorable response is equivalent to evaluating the likelihood that the subject will not exhibit favorable response (i.e., will exhibit a lack of response or be non-responsive).

Various aspects described herein are described in further detail in the following subsections.

### Antibodies

In some embodiments, the antibodies provided herein, or antigen binding fragments thereof, are recombinant antibodies that specifically bind to an HSV antigen, such as an HSV glycoprotein B (gB) or glycoprotein D (gD) protein, or a fragment thereof. In some embodiments, the recombinant antibodies comprise a heavy chain (or fragment thereof) from a natural occurring antibody. For example, the recombinant antibody can comprise a naturally occurring human heavy chain and an Fc region from another antibody or a non-naturally occurring Fc region and/or a light chain from another antibody or a non-naturally occurring light chain. In some embodiments, the recombinant antibodies of the present invention comprise a light chain (or fragment thereof) from a naturally occurring antibody. For example, the recombinant antibody can comprise a naturally occuring human light chain and an Fc region from another antibody or a non-naturally occurring Fc region and/or a heavy chain from another antibody or a non-naturally occurring heavy chain.

In some embodiments, the antibodies or antigen binding fragments thereof provided herein bind to an HSV antigen and include desired functional properties. For example, in some embodiments, the antibodies or antigen binding fragments thereof provided herein bind to a HSV gB or gD protein and block its binding to a host cell surface protein or other molecule on the host cell surface, thereby preventing entry of HSV into the cell

In some embodiments, antibodies of the present invention that bind to gB comprise a heavy chain or light chain sequence listed in Table 2. In some embodiments, the antibodies are recombinant antibodies. In some embodiments, the antibodies are isolated from a naturally occurring or a genetically modified cell (e.g. B cell).

TABLE 2
gB mAb Heavy Chain Sequences

| | | SEQ | | SEQ |
|---|---|---|---|---|
| mAb | Heavy Chain Sequence | ID NO | Light Chain Sequence | ID NO |
| HSVB | QITLKESGGGLVQPGGSLRLS | 1 | DIQVTQSPSSLSASVGDRV | 54 |
| 1.59 | CAASGFTFYSYVMTWVRQAP | | TITCRASRGISNYLAWFQQ | |
| | GKGLEWVSTISDDGRSTYYAE | | KPGKAPKSLIYAASSLQGG | |
| | SVEGRFTISRDNFKNAVHLQM | | VPPRFSGGGSGTDFTLTIS | |
| | SSLRAEDTAVYYCVKGLAAVG | | SLQPEDFATYYCQQYFGF | |
| | QGNYFGMDVWGPGTTVTVSS | | PLTFGGGTKVEIK | |
| HSVB | QITLKESGGGVVLPGRSLRLSC | 2 | EIVMTQSPSSLSASVGDRV | 55 |
| 1.60 | AASGFTFNNYAMYWVRQAPG | | TITCRASQDITKSVVWYQQ | |
| | KGLEYVAFISYDGMKTDYADA | | RLGKAPKLLVYGTSRLASG | |
| | VQGRFSISRDNSRETLWLQMN | | VPSRFSGSGYGTDYSLTIS | |
| | SLRADDTAVYFCARDYLLIPRT | | SLQPEDFATYYCQQYYSN | |
| | ASNAFDLWGQGTTVTVSS | | PLTFGGGTKVEIK | |
| HSVB | QVQLVQSGGGVVQPGRSLRL | 3 | EIVLTQSPSSLSASVGDRV | 56 |
| 1.79 | SCAASGFTFSNYAMYWVRQA | | TITCRASQDITNSLVWYQQ | |
| | PGKGLEYMAFISYDGTKKDYA | | RPGKAPNLLLYGASRLASG | |
| | DSVKGRFSISRDNSRETLWLQ | | VPSRFSGSGSGTDYSLTIS | |
| | MNSLRAEDTAVYFCARDYLLIP | | SLQPEDFATYYCQQYYRN | |
| | RTASNAFDLWGQGTTVTVSS | | PLTFGGGTKVDIK | |
| HSVB | EVQLVESGAELKKPGESLKISC | 4 | EIVMTQSPLSLPVTPGEPA | 57 |
| 1.97 | KGSGYSFSSHWIGWVRQMPG | | SISCRSSQSLLYSNGYNYL | |
| | KGLEWMGIIHPGDSDTRYSPS | | DWYLQKPGKSPQLLIYLGS | |
| | FQGQVTISVDKSITTAYLQWSS | | NRASGVPDRISGSGSGTD | |
| | LKASDTAIYYCAIQGYCRSGRC | | FTLKISRVEAEDVGVYYCM | |
| | PEGGDWFDPWGQGTLVTVSS | | QALQISYTFGQGTKVDIK | |
| HSVB | EVQLVESGGGVVQPGRSLRLS | 5 | DIVMTQTPSSLSASVGDRV | 58 |
| 1.105 | CAASGFTFSPYAMHWVRQAP | | TITCRASQDITKSIVWYQQK | |
| | GKGLQWVAFISYDGTKNDYAD | | AGKAPKVLLYASRLQSGVP | |
| | SVKGRFTISRDNSKNMVFLQM | | SRFSGSGSGTDYTLTISSL | |
| | NSLRPEDTALYYCARDILLIPRT | | QPEDFATYYCQQYYSNPL | |
| | TSYAFDIWGQGTTVTVSS | | TFGGGTKLEIK | |
| HSVB | QVQLVQSGGGLVQPGRSLRLS | 6 | DIRLTQSPDSLAVSLGERA | 59 |
| 1.107 | CVGSGFSFDDHGMHWVRQVP | | TINCKSRQSVLYSSINKNYL | |
| | GKGLQWVSGINWSSVLIGYVE | | AWYQQKPGQPPKLLIYWA | |
| | SVKGRFTISRDNAKNSLYLQM | | STRESGVPDRFSGSGSGT | |
| | NSLKPEDTALYFCARGPYSTS | | DFTLTISSLQAEDVAVYYC | |
| | QYFFDLWGQGTLVTVSS | | QQYYSLPYSFGQGTKLEIK | |
| HSVB | QVQLVQSGGGVVQPGRSLRL | 7 | EIVTTQSPSSLSASVGDRV | 60 |
| 1.108 | SCAASGFTFSPYAMHWVRQA | | TITCRASQDITKSIVWYQQK | |
| | PGKGLQWVAFISYDGTKNDYA | | AGKAPKVLLYASRLQSGVP | |
| | DSVKGRFTISRDNSKNMVFLQ | | SRFSGSGSGTDYTLTISSL | |
| | MNSLRPEDTALYYCARDILLIP | | QPEDFATYYCQQYYSNPL | |
| | RTTSYAFDIWGQGTTVTVSS | | TFGGGTKVDIK | |
| HSVB | EVQLVESGPEVKKPGASVKVS | 8 | EIVMTQSPATLSVSPGDRA | 61 |
| 1.111 | CKTSGYTFSTYGINWVRQAPG | | TLSCRASRSVTSNLAWYQ | |
| | HGLESLGWISAADGNINYPPKF | | HKPGQAPRLLIYGASTRAT | |
| | RGRVTMTRDTATSTVYMELRS | | GIPARFSGSGSGTEFTLTIS | |
| | LKPDDTAVYYCARDRATFGGP | | SLQSEDLAVYFCQQYNDW | |
| | LAAGDALDMWGQGIMVTVSS | | PRTFGQGTKVDIK | |
| HSVB | QVQLVQSGGGVVQPGRSLRL | 9 | DIVLTQTPSSLSASVGDRV | 62 |
| 1.116 | SCAASGFTFSNYAMYWVRQA | | TITCRASQDITNSLVWYQQ | |
| | PGKGLEYMAFISYDGTKKDYA | | RPGKAPNLLLYGASRLASG | |
| | DSVKGRFSISRDNSRETLWLQ | | VPSRFSGSGSGTDYSLTIS | |
| | MNSLRAEDTAVYFCARDYLLIP | | SLQPEDFATYYCQQYYRN | |
| | RTASNAFDLWGQGTTVTVSS | | PLTFGGGTKVEIK | |
| HSVB | QVTLKESGGGVVQPGRSLRLS | 10 | EIVMTQSPSSLSASVGDRA | 63 |
| 1.117 | CAASGFTFSNYAMYWVRQAP | | TITCRASQGITNSLAWYQQ | |
| | GKGLEYLAFISYDGTKKDYADS | | RPGKAPNLLLYGASRLARG | |
| | VKGRFSISRDNSKETLWLQMS | | VPSRFSGSGSGTDYSLTIS | |
| | SLRPEDTAVYFCARDYLLIPRT | | SLQPEDYATYYCQQYYNN | |
| | ASNAFDIWGQGTTVTVSS | | PLTFGGGTKVDIK | |
| HSVB | QVQLVESGGGVVQPGRSLRLS | 11 | DIQVTQSPSTLSASVGDRV | 64 |
| 1.121 | CVASIFIFENYGMHWVRQAPG | | TITCRASQSVDNWLAWYQ | |
| | KGLEWVAVISHDGRDQYYADS | | QKPGKAPKVVIWRVSALES | |
| | VKGRFSISRDNSKSTVYLQMN | | GVSPRFSGSGSGTEFTLTI | |
| | SLRPEDTAVYYCAKGVYGGHS | | NSLQPDDFATYFCQQYKT | |
| | IWDLFGDWGQGTLVTVSS | | YDLTFGGGTKVDIK | |
| HSVB | QVQLVQSGGGLVKPGGSLRLS | 12 | EIVMTQSPSSLSASVGDRV | 126 |
| 1.156 | CAASGFTFSDYYMTWIRQAPG | | TITCRASQGIRNDLGWYQQ | |
| | KGLEWVAYISSGGSSTYDADS | | KPGKAPKRLIYAASSLQSG | |
| | VKGRLTISRDNAKNSLYLQMN | | VPSRFSGSGSGTEFTLTIS | |
| | SLRAEDTAVHYCARVDMGPVS | | SLQPEDFATYYCLQHNSYP | |
| | SPIDYWGQGTLVTVSS | | YTFGQGTRLEIK | |
| HSVB | EVQLVESGGGLVKPGGSLRLS | 13 | EIVMTQSPSSLSASVGDR | 127 |
| 1.159 | CAASGFTFGDHYMSWVRQAP | | VTITCRASQGIRNDLGWYQ | |
| | GKGLEWLAYISTSATYRGYAD | | QKPGKAPKRLIYAASSLQS | |
| | SVKGRFTISRDNAKNSLYLQM | | GVPSRFSGSGSGTEFTLTI | |
| | DSLRAEDTAVYFCARDQDSSG | | SSLQPEDFATYYCLQHNSY | |
| | YYDAFDIWGQGTTVTVSS | | PYTFGQGTRLEIK | |
| HSVB | QVQLVQSGAEVKKPGASVQVS | 14 | EIVMTQSPSSLSASVGDR | 128 |
| 1.160 | CKATGYIFTNYYMHWVRLAPG | | VTITCRASQGIRNDLGWYQ | |
| | QGLEWMGKINLNGGSTAYAQK | | QKPGKAPKRLIYAASSLQS | |
| | FQGRVTMTRDTPTSTVNMELS | | GVPSRFSGSGSGTEFTLTI | |
| | SLTSEDTAIYYCARGSTSSSKY | | SSLQPEDFATYYCLQHNSY | |
| | GLDVWGQGTTVTVSS | | PYTFGQGTRLEIK | |
| HSVB | QVQLVQSGTEVKKPGESLKIS | 15 | EIVMTQSPSSLSASVGDR | 129 |
| 1.180 | CDVSGYTFTTYWIGWVRQKPG | | VTITCRASQGIRNDLGWYQ | |
| | KGLEWMGIIYPGDSDIRYSPSF | | QKPGKAPKRLIYAASSLQS | |
| | QGQVSISADKSISTAYLQWSSL | | GVPSRFSGSGSGTEFTLTI | |
| | KASDTAMYYCARRDGSGSGH | | SSLQPEDFATYYCLQHNSY | |
| | FDYWGQGTLVTVSS | | PYTFGQGTRLEIK | |
| HSVB | QVQLVQSGGGLVQPGGSLRL | 16 | DIVLTQSPGTLSLSPGERA | 65 |
| 1.225 | SCSAPGFSFSWYWMTWVRQA | | TLSCRASQSISSSYIAWYQ | |
| | PGKGLEWVANIKQDGSEKHYV | | QRPGQAPRLLIYGASTRAA | |
| | DSVEGRFTISRDNANNSLYLQ | | GIPGRFSGSGSGTDFTLTI | |
| | MNSLRAEDTAVYYCARGVRTV | | SRLEPEDFAVYFCQQYGT | |
| | YCGGACYPLGDWGQGTLVTV | | SPKTFGQGTKLEIK | |
| | SS | | | |
| HSVB | QVQLQQSGGGVVQPGRSLRL | 17 | DIVLTQSPLSLPVTLGQSAS | 66 |
| 1.227 | SCVGSGFTLRTYGMFWVRQA | | ISCRSSQGLVHSDGNSYLN | |
| | PGKGLEWLAFISYDGGIKYYAD | | WFHQRPGQSPRRLIYKVS | |
| | SVKGRFTISRDNSQNTLYLAIN | | FRDSGVPDRFSGSGSGTN | |
| | SLRTDDTGLYFCARDISGTYNA | | FTLKISRVEAEDVGVYYCM | |
| | WGQGPRSPSPQ | | QGTHWPPAFGQGTKXDIK | |
| HSVB | QVQLVQSGGGVVQPGRSLRL | 18 | DIRLTQSPSSLSASVGDRV | 67 |
| 1.229 | SCAASGFTFSNYAMYWVRQA | | TITCRASQGITNSLAWYQQ | |
| | PGKGLEYLAFISYDGTNKDYAD | | RPGKAPNLLLYGASRLARG | |
| | SVKGRFSISRDNSKETLWLQM | | VPSRFSGSGSGTDYSLTIS | |
| | SSLRPEDTAVYFCARDYLLIPR | | SLQPEDYATYYCQQYYNN | |
| | TASNAFDIWGQGTTVTVSS | | PLTFGGGTKVEIK | |
| HSVB | QVQLVQSGGGLVKPGGSLRLS | 19 | DIVMTQTPDSLAVSLGERA | 68 |
| 1.230 | CAASGFSFINAWMNWVRQAP | | TINCKSSQSVLYSSNNKNY | |
| | GKGLEWVGRIKSKTDGGTIEH | | LAWYQQKPGHPPKVLIYW | |
| | AAPVRGRFTISRDDSKNTLYLQ | | ASTRESGVPDRFSGSGSG | |
| | MTSLKIEDTAIYYCTTGSRGW | | TDFTLTISSLQAEDVAVYYC | |
| | WFDPWGQGTLVTVSS | | QQYYTAATFGGGTKLEIK | |
| HSVB | QVQLVQSGAEVKKPGASVKVS | 20 | EIVLTQSPSSLSASVGDRV | 69 |
| 1.231 | CKASGYTFTSYYMHWVRQAP | | TITCQASQDISNYLNWYQQ | |
| | GQGLEWMGIINPSGGGTTYAQ | | KPGKAPKLLIYDASNLETG | |
| | KFQGRVTMTRDTSTNTVYMEL | | VPSRFGGSGSGTDFTFTIS | |
| | ISLRSDDTAVYYCARDGTPILP | | SLQPEDIATYYCQQYDNLR | |
| | PSATTIGFDFWGQGTLVTVSS | | LTFGGGTKLEIK | |
| HSVB | QVQLVESGGGVVQPGRSLRLS | 21 | EIVMTQSPSSLSASVGDRV | 70 |
| 1.232 | CAASGFTFSNYAMYWVRQAP | | TITCRASQDITNSLVWYQQ | |
| | GKGLEYMAFISYDGTKKDYAD | | RPGKAPNLLLYGASRLASG | |
| | SVKGRFSISRDNSRETLWLQM | | VPSRFSGSGSGTDYSLTIS | |
| | NSLRAEDTAVYFCARDYLLIPR | | SLQPEDFATYYCQQYYRN | |
| | TASNAFDLWGQGTTVTVSS | | PLTFGGGTKVDIK | |
| HSVB | QVQLVQSGAEVKRPGASVKVS | 22 | DIRLTQSPSSLSASVGDRV | 71 |
| 1.234 | CKASGYTFISFAFHWVRQAPG | | TITCQASQDISNYLNWYQQ | |
| | QRLEWMGWINVANGNTKYSQ | | KPGKAPKLLIYDASNLETG | |
| | NFQGRVTITRDTSAGTAYMEL | | VPSRFGGSGSGTDFTFTIS | |
| | HSLRSEDTAVYFCATGDTGTH | | SLQPEDIATYYCQQYDNLR | |
| | YHYYAMDVWGQGTTVTVSS | | LTFGGGTKLEIK | |
| HSVB | QVTLKESGGGLVKPGGSLRLS | 23 | DIRLTQSPSSLSASVGDRV | 72 |
| 1.235 | CAASGFTFSSSTMTWVRQAP | | TITCQASQDISNYLNWYQQ | |
| | GKGLEWVSSISSSSSYIYYAAS | | KPGKAPKLLIYDASNLETG | |
| | VKGRFTISRDNPKNSLYLQMN | | VPSRFGGSGSGTDFTFTIS | |
| | SLRAEDTALYYCARAFGGNSE | | SLQPEDIATYYCQQYDNLR | |
| | RGFDYWGQGTLVTVSS | | LTFGGGTRLEIK | |
| HSVB | QVQLVQSGLEVKRPGASVKVS | 24 | EIVMTQSPATLSVSPGERA | 73 |
| 1.226 | CKASGHTFTNYGINWIRQTPG | | TLSCRASRSVTINLAWYQQ | |
| | QGLESLGWISASDGNINYARKF | | KPGQAPRLLIYGASTRATGI | |
| | RGRVTMTTDTSTSTVYMELRS | | PARFSGSGSGTEFTLTISS | |
| | LRSDDTAVYYCARDRATFEGIL | | LQSEDLAVYYCQQYNDWP | |
| | AARDAFDIWGQGTMVTVSS | | RTFGQGTKVEIK | |
| HSVB | QVQLVQSGPEVKKPGASVQVS | 25 | EIVLTQSPATLSVSPGESAT | 74 |
| 1.237 | CKAYGDTFMNLGFNWIRQAPG | | LSCRTSRSVTSNLAWYQQ | |
| | QGLESMGWISARDGNINYAPR | | KLGQAPRLLIYGASIRATGI | |
| | FRGRVTMTTDTSTSTVSVELR | | PARFSGSGSGTEFTLSISS | |
| | NLRADDTAVYFCATDRATFGGI | | LQSEDLGVYYCQQYNDWP | |
| | LAARDAFHIWGQGTTVTVSS | | RTFGQGTKLEIK | |
| HSVB | QVQLVQSGGGLIQPGGSLRLS | 26 | ETTLTQSPSSLSASVGDRV | 75 |
| 1.236 | CAASGFSVSSNYMTWVRQAP | | TITCQASQDISNYLNWYQQ | |
| | GKGLEWVSVIYAAGSTFYADS | | KPGKAPKLLIYDASNLETG | |
| | VKGRFTISRDNLTNTLSLQMNS | | VPSRFGGSGSGTDFTFTIS | |
| | LRADDTGVYFCARESVGDYNL | | SLQPEDIATYYCQQYDNLR | |
| | WHYYYGMDVWGQGTTVTVSS | | LTFGGGTKVEIK | |
| HSVB | EVQLVESGGGLVQPGGSLRLS | 27 | DIRLTQSPDSLAVSLGERA | 76 |
| 1.239 | CAASGFTFSSHWMSWVRQAP | | TFNCKSSQSVLYSSTNKNY | |
| | GKGPEWVANIKQDGSEKYYVD | | LAWYQQKPGLPPKLLIYWA | |
| | SVKGRFTISRDNVKNSLYLQM | | STRASGVPDRFSGSGSGT | |
| | NTLRAEDTAIYYCVREIYYDDN | | DFTLTISSLQPEDVAVYYC | |
| | SGFDYWGQGTLVTVSS | | HQYYTTPLTFGGGTKLEIK | |
| HSVB | QVQLVESGGGVVQPGRSLRLS | 28 | DIRLTQTPSSLSASVGDRV | 77 |
| 1.241 | CAASGFTFSNYAMYWVRQAP | | TITCRASQDITNSLVWYQQ | |
| | GKGLEYMAFISYDGTKKDYAD | | RPGKAPNLLLYGASRLASG | |
| | SVKGRFSISRDNSRETLWLQM | | VPSRFSGSGSGTDYSLTIS | |
| | NSLRAEDTAVYFCARDYLLIPR | | SLQPEDFATYYCQQYYRN | |
| | TASNAFDLWGQGTMVTVSS | | PLTFGGGTKVEIK | |
| HSVB | QVQLVQSGGGVVQPGRSLRL | 117 | DIQVTQSPSSLSASVGDRV | 130 |
| 1.238D. | SCAVSGFTFSPYAMHWVRQA | | TITCRASQDISNSLVWYQQ | |
| B | PGKGLEWVAYISSYEGTTNDY | | KAGQAPKILLYGSSRLQSG | |
| | ADSVKGRFTISRDNSKNTLYLQ | | VPSRFSGSGSGTDYTLTIS | |
| | MNSLRPEDTALYYCARDLFLIS | | GLLPEDFATYYCQQYYNN | |
| | RSSSYAFDLWGQGTLVTVSS | | PLTFGGGTKVDIK | |
| HSVB | QVQLVQSGGGVVQPGRSLRL | 118 | EIVLTQSPSSLSASVGDRV | 131 |
| 1.238D. | SCAVSGFTFSPYAMHWVRQA | | TITCRASQDISNSLVWYQQ | |
| A | PGKGLEWVAYISSYEGTTNDY | | KAGQAPKILLYGSSRLQSG | |
| | ADSVKGRFTISRDNSKNTLYLQ | | VPSRFSGSGSGTDYTLTIS | |
| | MNSLRPEDTALYYCARDLFLIS | | GLLPEDFATYYCQQYYNN | |
| | RSSSYAFDLWGQGTLVTVSS | | PLTFGGGTKLEIK | |
| HSVB | QVQLVQSGGGVVQPGRSLRL | 119 | DIQVTQSPSSLSASVGDRV | 132 |
| 1.238C. | SCAVSGFTFSPYAMHWVRQT | | TITCRASQDISNSLVWYQQ | |
| B | PGKGLEWVAYISSYEGTTNDY | | KAGQAPKILLYGSSRLQSG | |
| | ADSVKGRFTISRDNSKNTLYLQ | | VPSRFSGSGSGTDYTLTIS | |
| | MNSLRPEDTALYYRARDLFLIS | | GLLPEDFATYYCQQYYNN | |
| | RSSSYAFDLWGQGTMVTVSS | | PLTFGGGTKVDIK | |
| HSVB | QVQLVQSGGGVVQPGRSLRL | 120 | EIVLTQSPSSLSASVGDRV | 133 |
| 1.238C. | SCAVSGFTFSPYAMHWVRQT | | TITCRASQDISNSLVWYQQ | |
| A | PGKGLEWVAYISSYEGTTNDY | | KAGQAPKILLYGSSRLQSG | |
| | ADSVKGRFTISRDNSKNTLYLQ | | VPSRFSGSGSGTDYTLTIS | |
| | MNSLRPEDTALYYRARDLFLIS | | GLLPEDFATYYCQQYYNN | |
| | RSSSYAFDLWGQGTMVTVSS | | PLTFGGGTKLEIK | |
| HSVB | EVQLVESGGGVVQPGRSLRL | 121 | DIQVTQSPSSLSASVGDRV | 78 |
| 1.238B | SCAVSGFTFSPYAMHWVRQA | | TITCRASQDISNSLVWYQQ | |
| | PGKGLEWVAYISSYEGTTNDY | | KAGQAPKILLYGSSRLQSG | |
| | ADSVKGRFTISGDNSKNTLYLQ | | VPSRFSGSGSGTDYTLTIS | |
| | MNSLRPEDTALYYCARDLFLIS | | GLLPEDFATYYCQQYYNN | |
| | RSSSYAFDLWGQGTTVTVSS | | PLTFGGGTKVDIK | |
| HSVB | QVQLVESGGGVVQPGRSLRL | 122 | EIVLTQSPSSLSASVGDRV | 79 |
| 1.238A | SCAVSGFTFSPYAMHWVRQA | | TITCRASQDISNSLVWYQQ | |
| | PGKGLEWVAYISSYEGTTNDY | | KAGQAPKILLYGSSRLQSG | |
| | ADSVKGRFTISRDNSKNTLYLQ | | VPSRFSGSGSGTDYTLTIS | |
| | MNSLRPEDTALYYCARDLFLIS | | GLLPEDFATYYCQQYYNN | |
| | RSSSYAFDLWGQGTMVTVSS | | PLTFGGGTKLEIK | |
| HSVB | EVQLVESGAEVKKPGASVKVS | 29 | ETAFTQSPATLSLSPGERV | 80 |
| 1.242 | CKASGYTFTSYYIHWVRQAPG | | TLSCRASQSVGSSLAWYQ | |
| | QGPEWMGIINASGGSTSYAQK | | HKPGQAPRLLIYDASKRAT | |
| | FQGRVTMTRDTSTSTVYMELS | | GIPARFSGSGSGTDFTLTIS | |
| | SLRSEDTAVYYCARDGGGYYS | | SLEPEDFAVYYCQQCSNW | |
| | SGRSWFDPWGQGTLVTVSS | | PLTFGGGTKVEIK | |
| HSVB | VQLQESGPGLVKPSQTLSLTC | 30 | DIQMTQSPSSLSASVGDRV | 81 |
| 1.243 | TVSGASIISSGDYYWSWIRQPP | | TITCRASLSISSYVNWFQQ | |
| | GKGLEWVGYIYYSGSTYYNPS | | KPGKAPKFLIYAASSLQTG | |
| | LKSRVTISVDTSNNQFSLKLKS | | VPSRFSGGGSGTDFTLTIN | |
| | VTAADTAVYYCATLGPTVGYW | | SLQPEDFATYYCQQSYST | |
| | GQGTLVTVSS | | PYTFGQGTKVDIK | |
| HSVB | QVQLVQSGAEVKKPGASVKVS | 31 | DIVMTQSPDSLAVSLGERA | 82 |
| 1.249 | CKASGYTFSSYGISWVRQAPG | | TINCKSSLSVLYSSNNRNY | |
| | QGLEWMGWISLYNGNTNYAQ | | LAWYQQKLGQPPKLLFYW | |
| | NLQDRVTMTTGTSTSTAYLEL | | ASTRESGVPDRFSGSGSG | |
| | RSLRSDDTAMYFCARVGYGINI | | TDFTLTISNLQAEDVAVYY | |
| | LDRWGQGTLVTVSS | | CQQYYTAPWTFGQGTKVE | |
| | | | IK | |
| HSVB | QVQLQESGGGLVQPGGSLRL | 123 | DIQLTQSPSSLSASVGDRV | 83 |
| 1.259B | SCAASGFTFSSYWMSWVRQA | | TITCQASQDISNYLNWYQQ | |
| | PGKGLEWVANIKQDGSEKYYV | | KPGKAPKLLIYDASNLETG | |
| | DSVKGRLTISRDNAKNSLYLQ | | VPSRFGGSGSGTDFTFTIS | |
| | MNRLRAEDTAVYYCARDRGW | | SLQPEDIATYYCQQYDNLR | |
| | LEPTPWDYFDYWGQGTLVTVS | | LTFGGGTKLEIK | |
| | S | | | |
| HSVB | QVQLQESGGGLVQPGGSLRL | 32 | DIRMTQSPSSLSASVGDRV | 84 |
| 1.259 | SCAASGFTFSSYWMSWVRQA | | TITCQASQDISNYLNWYQQ | |
| | PGKGLEWVANIKQDGSEKYYV | | KPGKAPKLLIYDASNLETG | |
| | DSVKGRLTISRDNAKNSLYLQ | | VPSRFGGSGSGTDFTFTIS | |
| | MNRLRAEDTAVYYCARDRGW | | SLQPEDIATYYCQQYDNLR | |
| | LEPTPWDYFDYWGQGTLVTVS | | LTFGGGTKVEIK | |
| | S | | | |
| HSVB | QVQLVESGPGLVKPSQTLSLT | 33 | DIRVTQSPSSLSASVGDRV | 85 |
| 1.251 | CTVSGGSISSGGYYWSWIRQH | | TITCQASQDISNYLNWYQQ | |
| | PGKGLEWIGYIYYSGSTYYNPS | | KPGKAPKLLIYDASNLETG | |
| | LESRVTFSVDTSKDQFSLNLRS | | VPSRFGGSGSGTDFTFTIS | |
| | VTAADTAMYYCAPMTTVMSP | | SLQPEDIATYYCQQYDNLR | |
| | WGFDPWGQGTLVTVSS | | LTFGGGTKVDIK | |
| HSVB | QVQLVESGGGLVKPGGSLRLS | 34 | DIRVTQSPSSLSASVGDRV | 86 |
| 1.252 | CAASGFNFRSYGMNWVRQAP | | TITCQASQDISNYLNWYQQ | |
| | GKGLEWVSSISTSSSYKYYGD | | KPGKAPKLLIYDASNLETG | |
| | SVKGRFTISRDNAQQSVFLQM | | VPSRFGGSGSGTDFTFTIS | |
| | NSLRAEDTAIYFCARVDAEADA | | SLQPEDIATYYCQQYDNLR | |
| | LDCWGQGTLVTVSS | | LTFGGGTKLEIK | |
| HSVB | QVQLQQWGPGLVKPSGTLSLT | 35 | DIQVTQSPSSLSASVGDRV | 87 |
| 1.253 | CGVSGDSITSNNWWTWVRQP | | TITCQASQDISNYLNWYQQ | |
| | PGKGLEWIGEIYHTGNTNYNP | | KPGKAPKPLIYDASNLETG | |
| | SLQGRVALSVDKSKNHFSLTLT | | VPSRFGGSGSGTDFTFTIS | |
| | SVTAADTAFYFCARSWELRRL | | SLQPEDIATYYCQQYDNLR | |
| | YHDWGQGTLVTVSS | | LTFGGGTKVEIK | |
| HSVB | QVQLQQSGPGLVKPSQTLSLT | 36 | EIVLTQSPLSLPVTLGQPAS | 88 |
| 1.254 | CAISGDSVSSNSAAWNWIRQS | | ISCRSSQSLVHRDGNTYLT | |
| | PSRGLEWLGRTYYRSKWYND | | WFQQRPGQSPRRLVHKVS | |
| | YAESVKSRITVTPDTSKNQFSL | | NRDSGVPDRFSGSGSGTY | |
| | HLTSVTPEDTAVYYCGRDPPG | | FTLRISRVEAEDVGVYFCM | |
| | DQTIDVWGQGTTVTVSS | | QGTHRPYYNFGQGTKLEIK | |
| HSVB | QVQLVQSGGGVVQPGRSLRLS | 124 | EIRVTQSPSSLSASVGDRV | 89 |
| 1.256B | CAASGFTFSNYAMYWVRQAPG | | TITCRASQDITKSLVWYQQ | |
| | KGLEYMAFISYDGTIKDYADSVK | | RPGKAPNLLLYGASRLASG | |
| | GRFSISRDNSRETVWLQMNSLR | | VPSRFSGSGSGTDYRLTIS | |
| | PEDTAVYFCARDYLLIPRTASNA | | SLQPEDFATYYCQQYHRY | |
| | FDLWGQGTTVTVSS | | PLTFGGGTKVDIK | |
| HSVB | QVQLVQSGGGVVQPGRSLRL | 37 | ETTLTQSPDSLAVSLGERA | 90 |
| 1.256 | SCAASGFTFSNYAMYWVRQA | | TINCKSSQSVLYSFNSQNY | |
| | PGKGLEYMAFISYDGTIKDYAD | | LAWYQQKPGQPPKLLIYW | |
| | SVKGRFSISRDNSRETVWLQM | | ASTRESGVPVRFSGSGSG | |
| | NSLRPEDTAVYFCARDYLLIPR | | TDFTLTISSLQAEDVAVYYC | |
| | TASNAFDLWGQGTTVTVSS | | QQYVSTPLTFGQGTKVDIK | |
| HSVB | QVQLVQSGGGVVQPGRSLRL | 38 | EIVLTQSPSSLSASVGDRV | 91 |
| 1.258 | SCVASGFTFSRYAMHWVRQA | | TITCRASQGITNSLVWYQQ | |
| | PGKGLEWVAVISYDGSKKEYV | | KPGKAPKLLVYGTSRLESG | |
| | DSVKGRFAISRDNSKNMVYLQ | | VPSRFSGSGSGTDYTLTIS | |
| | MHSLRAEDTAVYTCARDVILVP | | SLQPEDFATYYCQQYYNY | |
| | AAISDAFDIWGQGTMVTVSS | | PLTFGGGTKVDIK | |
| HSVB | EVQLVESGGGVVQPGRSLRLS | 39 | DIVMTQSPDSLALSLGERA | 92 |
| 1.261 | CAASGFTFDSYGMHWVRQAP | | TINCKSSQSVLYSSNNKNY | |
| | GKGLEWVAVIWYDGSDKYYAD | | LAWYQQKPGQPPKLLIYW | |
| | SVKGRFTISRDNSKNILYLQMN | | ASTRESGVPDRFSGSGAG | |
| | SLRAEDTAVYYCAKGPLQDGH | | TDFTLTISSLQAEDVAVYYC | |
| | YFDYWGQGTLVTVSS | | QQYYGFPFTFGPGTKVEIK | |
| HSVB | QVQLVQSGGEVKKPGASVKVS | 40 | DIQVTQSPSSLSASVGDRV | 93 |
| 1.264 | CKTSGYTFTNYGITWVRQAPG | | TITCQASQDISNYLNWYQQ | |
| | RGLEWMGWISVYTGYTNYAQ | | KPGKAPKLLIYDASNLETG | |
| | KLQGRVTMTTDTSTRTAYMEL | | VPSRFGGSGSGTDFTFTIS | |
| | RSLRSDDTALYYCARVQEGHS | | SLQPEDIATYYCQQYDNLR | |
| | GRFDPWGQGTLVTSPQ | | LTFGGGTKVDIK | |
| HSVB | QVQLVQSGAEVKKPGASVRVS | 41 | DIRMTQSPSSLSASVGDRV | 94 |
| 1.265 | CKASGYNFAHFDLHWVRQAP | | TITCQASQDISNYLNWYQQ | |
| | GQGPEWMGWIHAGTGDIKYS | | KPGKAPKLLIYDASNLETG | |
| | QKFQGRVIITRDTAASTAYMEV | | VPSRFGGSGSGTDFTFTIS | |
| | TSLRSEDMAVYYCASLPPQEN | | SLQPEDIATYYCQQYDNLR | |
| | LWGQGTTVTVSS | | LTFGGGTKVDIK | |
| HSVB | EVQLVESGAEVKKPGESLKISC | 42 | EIVMTQSPDSLAVSLGERA | 95 |
| 1.269 | KGSGYSFTNYWVAWVRQVPG | | TINCKSSQSVLYSSNNKNY | |
| | KGLEWMGTIYPDDSDTRYSPS | | LSWYQQKPGQPPKLLIFW | |
| | FQGQVAISVDLSVNTAYVQWS | | ASTRESGVPDRFSGSGSE | |
| | SLKASDTAMYYCARPSPHNNA | | TDFTLTISSLQAEDVAVYYC | |
| | WKGFDIWGQGTTVTVSS | | QQYFDTPLTFGGGTKVEIK | |
| HSVB | QVQLVESGGGLAQPGGSLRLS | 43 | ETTLTQSPATLSLSPGERA | 96 |
| 1.270 | CAASGFTFDNYAMAWVRQAP | | TLSCRATQSIINYLAWYQQ | |
| | GKGLEWVSVISGSGTDTFYAD | | KHGQPPRLLIYDASHRATG | |
| | SVNGRFTISRDNSKTTLYLQVS | | IPARFSGSGSGTDFTLTISS | |
| | SLRAEDTALYYCARDATTPQG | | LEPEDFAVYYCQQRGNWP | |
| | LFDYWGQGTLVTVSS | | PTFGGGTKVEIK | |
| HSVB | QVQLVQSGAEVKKPGASVKVS | 44 | EIVMTQSPATLSLSPGERA | 134 |
| 1.272 | CKASGYTFTSYYMHWVRQAP | | TLSCWASQNIMGYLAWYQ | |
| | GQGLEWMGIINPSGGSTSYAQ | | QKPGQAPRLLIYDAFNRAT | |
| | KFQGRVTMTRDTSTSTVYMEL | | GVPAKFSGSGSGTDFTLTI | |
| | SSLRSEDTAVYYCARDSGVVVI | | SSLEPEDFAVYYCQQRSS | |
| | TWDYWGQGTLVTVSS | | WPLTFGGGTKVEIK | |
| HSVB | QVQLVQSGAEVKKPGASVKVS | 125 | EIVMTQSPSSLSASVGDRV | 135 |
| 1.272B | CKASGYTFTSYYMHWVRQAP | | TITCQASQDISNYLNWYQQ | |
| | GQGLEWMGIINPSGGSTSYAQ | | KPGKAPKLLIYDASNLETG | |
| | KFQGRVTMTRDTSTSTVYMEL | | VPSRFGGSGSGTDFTFTIS | |
| | SSLRSEDTAVYYCARDSGVVVI | | SLQPEDIATYYCQQYDNLR | |
| | TWDYWGQGTLVTVSS | | LTFGGGTKVDIK | |
| HSVB | QVQLVQSGAEVKKPGSSVKVS | 45 | DIQVTQSPSSLSPSVGDRV | 97 |
| 1.274 | CKASGGTFSSYAISWVRQAPG | | TITCQASQDISNYLNWYQQ | |
| | QGLEWMGGIIPMVGSAKSAKK | | KPGKAPKLLIYDASNLETG | |
| | FQGRVTITADASTSTAYMELGS | | VPSRFGGSGSGTDFTFTIS | |
| | LTSDDTAVYYCAREEQWLIRAF | | SLQPEDIATYYCQQYDNLR | |
| | DIWGQGTTVTVSS | | LTFGGGTKVDIK | |
| HSVB | QVQLVQSGAEVKKPGSSVKVS | 46 | DIVMTQSPSSLSASVGDRV | 98 |
| 1.276 | CKASGGTFSSYAISWVRQAPG | | TITCQASQDISNYLNWYQQ | |
| | QGLEWMGGIIPMVGSAKSAKK | | KPGKAPKLLIYDASNLETG | |
| | FQGRVTITADASTSTAYMELGS | | VPSRFGGSGSGTDFTFTIS | |
| | LTSDDTAVYYCAREEQWLIRAF | | SLQPEDIATYYCQQYDNLR | |
| | DIWGQGTTVTVSS | | LTFGGGTKVDIK | |
| HSVB | EVQLVESGGGLVQPGGSLRLS | 47 | EIVMTQSPLFLPVTLGQPA | 99 |
| 1.244 | CAASGFTFSAYWMTWVRQAP | | SISCRSSQSLVHSDGNTYL | |
| | GKGLEWVANIDKPGTKKYYVA | | SWFQQRPGQSPRRLIYKV | |
| | SIEGRFTISRDNAKNSLHLQMD | | SNRDSGVPDRFGGSGSGT | |
| | YLRGEDTAVYYCVRDHRGDPD | | DFTLKISRVEAEDVGVYYC | |
| | TSAWGQGTLVTVSS | | MQATHWPFTLGPGTKLEIK | |
| HSVB | QVQLVQSGGGVVQAGRSLRL | 48 | EIVMTQSPATLSLSPGERA | 100 |
| 1.246 | SCAASGFSFSNYGMHWVRQA | | TLSCWASQNIMGYLAWYQ | |
| | PGKGLEWVAVIWYDGSKKYYA | | QKPGQAPRLLIYDAFNRAT | |
| | ESVKGRFTISRDNSKSTVYLQ | | GVPAKFSGSGSGTDFTLTI | |
| | MNSLRVEDTAVYYCARDGYRR | | SSLEPEDFAVYYCQQRSS | |
| | LDYYGMDVWGQGTTVTVSS | | WPLTFGGGTKVEIK | |
| HSVB | QITLKESGPGLVKPSQTLSLTC | 49 | EIVMTQSPGTLSLSPGERA | 101 |
| 1.247 | TVSGGSISSAEHYWSWIRQSP | | TLSCRASQSVHNTFLAWY | |
| | GKGLEWIGYIYYSGTTYYNPSL | | QQRFGQAPRLLIYGASSRA | |
| | ESRVTISLDTSKSQFSLKLSSLT | | TGIPDRFSGSGSGTDFTLTI | |
| | STDTAVYYCARASYCVGGSRP | | NRLEAEDFAVYYCQQYSN | |
| | FDPWGQGTMVTVSS | | PPFTFGGGTKVDIK | |
| HSVB | QVQLVESGGGLVQPGGSLRLS | 50 | DIVMTQSPSSLSASVGDRV | 102 |
| 1.248 | CAASGFTFTNYVMHWVRQAP | | TITCQASQDISNYLNWYQQ | |
| | GKGLEWVSGIGTVGDTYYLGS | | KPGKAPKLLIYDASNLETG | |
| | VKGRFTISRESAKKSLYLQMNS | | VPSRFGGSGSGTDFTFTIS | |
| | LRAGDTAVYYCARGGGGSDSL | | SLQPEDIATYYCQQYDNLR | |
| | HWDTVIDDWGQGTLVTVSS | | LTFGGGTKLEIK | |
| HSVB | QVTLKESGPGLVKPSQTLSLTC | 51 | EIVLTQSPGTLSLSPGDRA | 103 |
| 1.277A | IVSGGSISGYFWSWIRQHPGK | | ALTCRTSQSISYRQLAWYQ | |
| | GLEWIGYVHYSGSTYYNPSLK | | HKPGQPPRLLIHGASNRAT | |
| | SRVTISVDTSKNQFSLKLTSVT | | GIPDRFSGSGSGTDFTLTIS | |
| | AADTAVYYCARASTSGGFDPW | | RLEPEDFAVFYCQQYSTSP | |
| | GQGTLVTVSQ | | PTFGGGTKVEIK | |
| HSVB | QVQLVQSGAEVKKPGASVTVS | 52 | EIVLTQSPVSLSVTLGQPA | 104 |
| 1.277B | CKASGYSFRSYHVHWVRQAP | | SISCRSTHSLDYSDGNTYL | |
| | GQGLEWVAWIDADAADTIYAQ | | NWFHQRPGHSPRRLIYTV | |
| | KFQGRVTVTRDTSTSTVHMVL | | SDRDSGVPDRFSGSGSGT | |
| | SGLKSDDTAVYYCARAPTIAFY | | DFTLKISRVEAEDVGVYYC | |
| | FDHWGLGTLVTVSS | | MQGTHWPYTFGXGXKVEI | |
| | | | K | |
| HSVB | QVQLVQSGGGLVQPGGSLRL | 53 | DIVLTQSPVSLSVTLGQPA | 105 |
| 1.279 | SCAASGFTLSDYWMNWVRQA | | SISCRSTHSLDYSDGNTYL | |
| | PGRGLEWVASIKADGSEKYYV | | NWFHLRPGHSPRRLIYTVS | |
| | DSVTGRFSISRDSGKNSLYLQ | | DRDSGVPDRFSGSGSGTD | |
| | MNSLTAEDTAIYYCVRGLSGID | | FTLKISRVEAEDVGVYYCM | |
| | WGXGTTVTVSS | | QGTHWPYTFGQGTKVEIK | |
| HSVB | QVQLVQSGGGLIQPGESLRLS | 153 | EIVMTQSPLSLPVTPGEPA | 106 |
| 1.280 | CAVSGFTVNNNYISWVRQAPG | | SISCRSGQSLLHSNGYNYL | |
| | KALEWVSVIYHGARAYYADSV | | DWYLQKPGQSPQLLIYLGS | |
| | KGRFTISIDNSKNTLYLQMNSL | | NRASGVPDRFSGSGSGTD | |
| | RAEDTAVYYCARDRGSGDMD | | FTLKISRVEADDVGVYYCM | |
| | AWGQGTTVTVSS | | QALQTPYTFGQGTKVDIK | |

*X = any of the 20 amino acids

In some embodiments, monoclonal antibodies of the present invention that bind to gD comprise a heavy chain or light chain sequence listed in Table 3.

TABLE 3
gD mAb Heavy and Light

| | | SEQ | | SEQ |
|---|---|---|---|---|
| mAb | Heavy Chain | ID NO | Light Chain | ID NO |
| HSVD | EVQLLESGAEVKKPGSSVKVSC | 136 | DIQLTQSPATLSVSPGERAT | 141 |
| 1.4 | QASGGTFSTYAVSWVRQAPGQ | | LSCRASQSVSGNLAWYQHK | |
| | GLEWMGGIIPIFGTANYAQKFQ | | PGQAPRLLIYGASTRATGIP | |
| | GRVTITADKSTNTAYMEMSRLR | | ARFSGSGSGTEFTLTISCLQ | |
| | SEDTAVYYCARVAGRGTVVTP | | SEDFAVYYCQQYNNWVTF | |
| | WNHFDYWGQGTLVTVSS | | GGGTKVEIK | |
| HSVD | QVQLVQSGAEVKKPGSSVKVS | 137 | DIVMTQSPSTLSASVGDRVT | 143 |
| 1.13 | CQASGGTFSTYAVSWVRQAPG | | ITCRASQSINSWSAWYQQK | |
| | QGLEWMGGIIPIFGTANYAQKF | | PGKAPKPLIYQASSLESGVP | |
| | QGRVTITADKSTNTAYMEMSRL | | SRFSGSGSGTEFTLTISSLQ | |
| | RSEDTAVYYCARVAGRGTVVTP | | PDDFATYYCQQYNSYSSYT | |
| | WNHFDYWGQGTLVTVSS | | FGQGTKVDIK | |
| HSVD | QVTLKESGAAVKKPGSSVTVSC | 107 | DIXMTQSPGTLSLSPGERAA | 112 |
| 1.2 | KASGGSFSTYAMTWVRQAPGQ | | LSCRASQSVSSTYLAWYQQ | |
| | GLEWMGGITPVFGIVDYAQKFQ | | KPGQAPRLLIYGTSSRATGI | |
| | GRVTITADASTSTAYMELTSLRS | | PDRFSGSGSGTDFTLTISRL | |
| | EDSAVYYCARIGYCTAGDCSIPR | | EPEDFAVYYCQQYGSSALT | |
| | GAFDIWGQGTMVTVSS | | FGGGTKVDIK | |
| HSVD | QVQLVESGAEVRKPGSSVKVSC | 138 | DIVMTQTPGTLSLSPGERAT | 144 |
| 1.5 | KASGGTFSSYAISWVRQAPGQ | | LSCRASQSVSGNYLAWYQ | |
| | GLEWMGGIIPIFGTPNYAQRFQ | | QKPGQAPRLLIYGASSRAT | |
| | DRVTITADASTSTAYMELSSLRS | | GIPDRFSDSGSGTDFTLTIS | |
| | DDTAVYYCAREGYCLGSTCHLS | | RLEPEDFAVYYCQQYGSSP | |
| | GGGAFDIWGLGTTVTVSS | | LTFGGGTKVDIK | |
| HSVD | EVQLLESGGGVVQPGGSLRLSC | 108 | DIQVTQSPSSLSASVGDRVT | 113 |
| 1.14 | AASGFSFSDYWMHWVRQVPG | | FTCRASQSVDTYLNWYQQK | |
| | QGLEWVSRIETDGSSTTYVDSV | | PGKAPKLLIYTASSLQLGVP | |
| | KGRFTISRDNANNTLFLQMNSL | | SRFSGRGAGTEFTLTISSLQ | |
| | RAEDTAVYYCARDEGPFYYDFS | | PEDFATYYCQQTYNTPHTF | |
| | SGFSDGMDVWGQGTTVTVSS | | GQGTKVEIK | |
| HSVD | QVQLVQSGAEVKKPGASVKVS | 109 | DIQLTQSPSSLSASVGDRVT | 114 |
| 1.15 | CKASGYSFISYYMHWVRQAPG | | ITCRASQSISTYLNWYQQKP | |
| | QGLEWMGIINPSGGSTSYAQNF | | GRAPNLLIYDASSVQSGVPS | |
| | QGRVTMTSDTSTSTVYMELSSL | | RFSGSGSGTDFTLTISSLQP | |
| | RSEDTAVYYCARDRLRKDAYG | | DDFVTYYCQQSYSSPRTFG | |
| | MDVWGQGTTVTVSS | | QGTKVDIK | |
| HSVD | QVTLKESGAEVKKPGSSVKVSC | 139 | DIQMTQSPSSLSASVGDRV | 145 |
| 1.18 | KASEGTIRTYAVTWLRQAPGQR | | TITCKASQDVTTAVAWYQQ | |
| | LEWMGGIIPIFGKVNHAERFQG | | KPGKAPKLLIYWASTRHTGV | |
| | RVAITADELTGTVYMELSSLTSQ | | PSRFSGSGSGTDFTLTISSL | |
| | DTAMYFCARDLMLANSPPAFDL | | QPEDFATYYCQQHYSTPLT | |
| | WGQGTLVTVSS | | FGQGTKVEIK | |
| HSVD | EVQLVESGAEVKKPGASVRASC | 140 | DIVMTQSPATLSVSPGEGAT | 146 |
| 1.19 | KTSGYPFTSYYIHWVRQAPGQG | | LSCRASQSVGSNLAWYQHK | |
| | LEWMGMINPSGGSTRYAQKFQ | | PGQPPRLLIYGASARATSIP | |
| | GRVTMTSDTSTSTVYMEMSSLR | | AKFSGSGSGTDFTLTISSLQ | |
| | SVDTAVYYCARSSPTGYSTNRG | | SEDFAVYYCQQYNNWPLTF | |
| | RGYSGIDVWGQGTTVTVSS | | GGGTKVDIK | |
| HSVD | QVQLVESGAEVKEPGASVKVSC | 110 | ETTLTQSPDSLAVSLGERAT | 115 |
| 1.22 | KASGYTFTSFTIHWVRQAPGQS | | INCKSSQSVLYSFNSQNYLA | |
| | LEWMGWINAGNGYTKYSQRFQ | | WYQQKPGQPPKLLIYWAST | |
| | DRLTITSDTSANTAYMDLRSLGS | | RESGVPVRFSGSGSGTDFT | |
| | EDTALYYCARGRGDRRIVVLQP | | LTISSLQAEDVAVYYCQQYV | |
| | FEALDFWGQGTTVTVSS | | STPLTFGQGTKVDIK | |
| HSVD | QVQLVQSGAEVRKPGSSVKVS | 111 | ETMMTQTPASLSVSPGERAI | 116 |
| 1.24 | CKASGGTFHSYTINWVRQAPG | | LSCWASQSVGRNLAWYQH | |
| | QGLQVLGGILPVFGTTNYAQKF | | KPGQAPRLLVYAASARATG | |
| | QDRVIITADASTSTAYMELSSLR | | VPARFSGGGSGTDFTLTISS | |
| | SEDTAVYYCAKLAGQPIVGSDY | | LQSEDFAVYYCQQYDKWP | |
| | YYDSWGQGTLVTVSS | | GTFGQGTKVDIK | |

*X = any of the 20 amino acids

In some embodiments, the amino acid sequence of the heavy chain of an antibody of the present invention can have about 80%, 81%, 82%, 83%, 84%, 85%, 86%, 87%, 88%, 89%, 90%, 91%, 92%, 93%, 94%, 95%, 96%, 97%, 98%, 99%, or even greater sequence identity to a heavy chain amino acid sequence in Table 2 or 3. In some embodiments, the amino acid sequence of the light chain of an antibody of the present invention can have about 80%, 81%, 82%, 83%, 84%, 85%, 86%, 87%, 88%, 89%, 90%, 91%, 92%, 93%, 94%, 95%, 96%, 97%, 98%, 99%, or even greater sequence identity to a light chain amino acid sequence in Table 2 or 3.

In some embodiments, the antibodies or antigen binding fragments thereof provided herein block entry of HSV into a cell. In some embodiments, the antibodies or antigen binding fragments thereof provided herein reduce HSV infection.

Also provided are modified antibodies and/or antigen binding fragments which can be prepared using an antibody having one or more of the V_{H} and/or V_{L} sequences disclosed herein as starting material to engineer a modified antibody which may have altered properties from the starting antibody. An antibody can be engineered by modifying one or more residues within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al. (1998) Nature 332:323-327: Jones, P. et al. (1986) Nature 321:522-525: Queen, C. et al. (1989) Proc. Natl. Acad. See. U.S.A. 86:10029-10033: U.S. Pat. No. 5,225,539 to Winter, and U.S. Pat. Nos. 5,530,101; 5,585,089; 5,693,762 and 6,180,370 to Queen et al.)

Such framework sequences can be obtained from public DNA databases or published references that include germline antibody gene sequences. For example, germline DNA sequences for human heavy and light chain variable region genes can be found in the “VBase” human germline sequence database (available on the Internet at www.mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242: Tomlinson, I. M., et al. (1992) “The Repertoire of Human Germline V_{H} Sequences Reveals about Fifty Groups of V_{H} Segments with Different Hypervariable Loops” J. Mol. Biol. 227:776-798; and Cox, J. P. L. et al. (1994) “A Directory of Human Germ-line V_{H} Segments Reveals a Strong Bias in their Usage” Eur. J. Immunol. 24:827-836; the contents of each of which are expressly incorporated herein by reference.

Preferred framework sequences for use in the antibodies described herein are those that are structurally similar to the framework sequences used by antibodies described herein. The V_{H} CDR1, 2 and 3 sequences, and the V_{L} CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al).

Engineered antibodies described herein include those in which modifications have been made to framework residues within V_{H} and/or V_{L}, e.g. to improve the properties of the antibody. Typically such framework modifications are made to decrease the immunogenicity of the antibody. For example, one approach is to “backmutate” one or more framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “backmutated” to the germline sequence by, for example, site-directed mutagenesis or PCR-mediated mutagenesis. Such “backmutated” antibodies are also intended to be encompassed.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

Another type of variable region modification is to mutate amino acid residues within the VH and/or VL CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples. Preferably conservative modifications (as discussed above) are introduced. The mutations may be amino acid substitutions, additions or deletions, but are preferably substitutions. Moreover, typically no more than one, two, three, four or five residues within a CDR region are altered.

### Nucleic Acids, Vectors, and Host Cells

The nucleotide sequences corresponding to (e.g., encoding) the anti-HSV antibodies and antigen binding fragments disclosed herein include all degenerate sequences related to the disclosed antibodies, i.e., all nucleic acids having a sequence that encodes one particular peptide and variants and derivatives thereof.

Typically, said nucleic acid is a DNA or RNA molecule, which may be included in any suitable vector, such as a plasmid, cosmid, episome, artificial chromosome, phage or a viral vector.

Such vectors can be either circular or linear. Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

The provided vectors also can include, for example, origins of replication and/or markers. A marker gene can confer a selectable phenotype, e.g., antibiotic resistance, on a cell. The marker product is used to determine if the vector has been delivered to the cell and once delivered is being expressed. Examples of selectable markers for mammalian cells are dihydrofolate reductase (DHFR), thymidine kinase, neomycin, neomycin analog G418, hygromycin, puromycin, and blasticidin. When such selectable markers are successfully transferred into a mammalian host cell, the transformed mammalian host cell can survive if placed under selective pressure. Examples of other markers include, for example, the E. coli lacZ gene, green fluorescent protein (GFP), and luciferase. In addition, an expression vector can include a tag sequence designed to facilitate manipulation or detection (e.g., purification or localization) of the expressed polypeptide. Tag sequences, such as GFP, glutathione S-transferase (GST), polyhistidine, c-myc, hemagglutinin, or FLAG™ tag (Kodak: New Haven, CT) sequences typically are expressed as a fusion with the encoded polypeptide. Such tags can be inserted anywhere within the polypeptide including at either the carboxyl or amino terminus.

In some instances, the disclosure includes cells comprising the nucleic acids (e.g., vectors) and/or peptides disclosed herein. Both prokaryotic and eukaryotic host cells, including insect cells, can be used as long as sequences requisite for maintenance in that host, such as appropriate replication origin(s), are present. For convenience, selectable markers are also provided. Host systems are known in the art and need not be described in detail herein. Prokaryotic host cells include bacterial cells, for example, E. coli, B. subtilis, and mycobacteria. Among eukaryotic host cells are yeast, insect, avian, plant, C. elegans (or nematode) and mammalian host cells. Examples of fungi (including yeast) host cells are S. cerevisiae, Kluyveromyces lactis (K. lactis), species of Candida including C. albicans and C. glabrata, Aspergillus nidulans, Schizosaccharomyces pombe (S. pombe), Pichia pastoris, and Yarrowia lipolytica. Examples of mammalian cells are COS cells, mouse L cells, LNCaP cells, Chinese hamster ovary (CHO) cells, human embryonic kidney (HEK) cells, and African green monkey cells. Xenopus laevis oocytes, or other cells of amphibian origin, may also be used.

In general, cells that can be used herein are commercially available from, for example, the American Type Culture Collection (ATCC), P.O. Box 1549, Manassas, VA 20108. See also F. Ausubel et al., Current Protocols in Molecular Biology, John Wiley & Sons, New York, NY, (1998). Transformation and transfection methods useful in the generation of the cells disclosed herein are described, e.g., in F. Ausubel et al., Current Protocols in Molecular Biology, John Wiley & Sons, New York, NY, (1998).

In some instances, the disclosed therapeutic compositions can include a vector (e.g., expression vector, a viral vector, an adeno-associated virus vector) comprising a nucleic acid encoding and an antibody or antigen binding fragment thereof described herein. As described herein, antibodies and antibody fragments include, but are not limited to, monoclonal antibodies (including full-length monoclonal antibodies), polyclonal antibodies, multispecific antibodies formed from at least two different epitope binding fragments (e.g., bispecific antibodies), camelised antibodies, chimeric antibodies, single-chain Fvs (scFv), single-chain antibodies, single domain antibodies, domain antibodies, Fab fragments, F(ab′)2 fragments, antibody fragments that exhibit the desired biological activity (e.g. the antigen binding portion), disulfide-linked Fvs (dsFv), and anti-idiotypic (anti-Id) antibodies (including, e.g., anti-Id antibodies to antibodies of the invention), intrabodies, and epitope-binding fragments of any of the above.

### Methods of Producing Antibodies

Antibodies and fragments thereof, immunoglobulins, and polypeptides of the present invention may be produced by any technique known in the art, such as, without limitation, any chemical, biological, genetic or enzymatic technique, either alone or in combination.

Knowing the amino acid sequence of the desired sequence, one skilled in the art can readily produce said antibodies or polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies and other polypeptides of the present invention can be synthesized by recombinant DNA techniques as is well-known in the art. For example, these fragments can be obtained as DNA expression products after incorporation of DNA sequences encoding the desired (poly) peptide into expression vectors and introduction of such vectors into suitable eukaryotic or prokaryotic hosts that will express the desired polypeptide, from which they can be later isolated using well-known techniques.

In particular, the present invention further relates to a method of producing an antibody or a polypeptide of the invention, which method comprises the steps consisting of: (i) culturing a transformed host cell according to the invention under conditions suitable to allow expression of said antibody or polypeptide; and (ii) recovering the expressed antibody or polypeptide.

Antibodies and other polypeptides of the present invention are suitably separated from the culture medium by conventional immunoglobulin purification procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Methods for producing antibody fragments are well-known in the art. For example, Fab fragments can be obtained by treating an antibody with a protease such as papain. Also, Fabs can be produced by inserting DNA encoding Fabs of the antibody into a vector for prokaryotic expression system, or for eukaryotic expression system, and introducing the vector into a procaryote or eucaryote (as appropriate) to express the Fabs.

Similarly, F(ab′) 2 fragments of the present invention can be obtained treating an antibody with a protease, pepsin. Also, the F(ab′)2 fragment can be produced by binding Fab′ described below via a thioether bond or a disulfide bond.

Fab′ fragments can be obtained treating F(ab′)2 with a reducing agent, dithiothreitol. Also, the Fab′ fragments can be produced by inserting DNA encoding a Fab′ fragment of the antibody into an expression vector for prokaryote, or an expression vector for eukaryote, and introducing the vector into a prokaryote or eukaryote (as appropriate) to perform its expression.

In addition, scFvs of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv.

### Pharmaceutical Formulations

In some instances, the antibodies and/or antigen-binding fragments disclosed herein can be formulated for use as or in pharmaceutical compositions. Such compositions can be formulated or adapted for administration to a subject via any route, e.g., any route approved by the Food and Drug Administration (FDA). Exemplary methods are described in the FDA's CDER Data Standards Manual, version number 004 (which is available at fda.give/cder/dsm/DRG/drg00301.htm).

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

In some instances, pharmaceutical compositions can include an effective amount of one or more peptides. The terms “effective amount” and “effective to treat,” as used herein, refer to an amount or a concentration of one or more peptides disclosed herein (e.g., antibody or antibody fragment described herein) for a period of time (including acute or chronic administration and periodic or continuous administration) that is effective within the context of its administration for causing an intended effect or physiological outcome.

In some instances, pharmaceutical compositions can include one or more peptides and any pharmaceutically acceptable carrier, adjuvant, and/or vehicle. In some instances, pharmaceuticals can further include one or more additional therapeutic agents in amounts effective for achieving a modulation of disease or disease symptoms.

The term “pharmaceutically acceptable carrier or adjuvant” refers to a carrier or adjuvant that may be administered to a patient, together with a peptide of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-I-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat. Cyclodextrins such as I-, [symbol] -, and K-cyclodextrin, may also be advantageously used to enhance delivery of compounds of the formulae described herein.

The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases, or buffers to enhance the stability of the formulated compound or its delivery form. The term parenteral as used herein includes subcutaneous, intracutaneous, intravenous, intramuscular, intra-articular, intraarterial, intrasynovial, intrasternal, intrathecal, intralesional, and intracranial injection or infusion techniques.

Compositions or pharmaceutical compositions comprising the antibodies, ScFvs or fragments of antibodies disclosed herein are preferably comprise stabilizers to prevent loss of activity or structural integrity of the protein due to the effects of denaturation, oxidation, or aggregation over a period of time during storage and transportation prior to use. The compositions or pharmaceutical compositions can comprise one or more of any combination of salts, surfactants, pH, and tonicity agents such as sugars can contribute to overcoming aggregation problems. Where a composition or pharmaceutical composition of the present invention is used as an injection, it is desirable to have a pH value in an approximately neutral pH range, it is also advantageous to minimize surfactant levels to avoid bubbles in the formulation which are detrimental for injection into subjects. In an embodiment, the composition or pharmaceutical composition is in liquid form and stably supports high concentrations of bioactive antibody in solution and is suitable for inhalational or parenteral administration. In an embodiment, the composition or pharmaceutical composition is suitable for intravenous, intramuscular, intraperitoneal, intradermal and/or subcutaneous injection. In an embodiment, the composition or pharmaceutical composition is in liquid form and has minimized risk of bubble formation and anaphylactoid side effects. In an embodiment, the composition or pharmaceutical composition is isotonic. In an embodiment, the composition or pharmaceutical composition has a pH or 6.8 to 7.4.

Examples of pharmaceutically acceptable carriers include, but are not limited to, phosphate buffered saline solution, sterile water (including water for injection USP), emulsions such as oil/water emulsion, and various types of wetting agents. Preferred diluents for aerosol or parenteral administration are phosphate buffered saline or normal (0.9%) saline, for example 0.9% sodium chloride solution, USP. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, the content of each of which is hereby incorporated in its entirety). In non-limiting examples, the can comprise one or more of dibasic sodium phosphate, potassium chloride, monobasic potassium phosphate, polysorbate 80 (e.g. 2-[2-[3,5-bis(2-hydroxyethoxy)oxolan-2-yl]-2-(2-hydroxyethoxy) ethoxy]ethyl (E)-octadec-9-enoate), disodium edetate dehydrate, sucrose, monobasic sodium phosphate monohydrate, and dibasic sodium phosphate dihydrate.

In an embodiment, antibodies, or fragments of antibodies, or compositions, or pharmaceutical compositions described herein are lyophilized and/or freeze dried and are reconstituted for use. The antibodies, or fragments of antibodies, or compositions, or pharmaceutical compositions described herein can also be provided in any suitable forms including, but not limited to, injectable solutions or inhalable solutions, gel forms, and tablet forms.

In some embodiments, pharmaceutical compositions can be in the form of a solution or powder for inhalation and/or nasal administration. Such compositions may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

In some embodiments, pharmaceutical compositions can be orally administered in any orally acceptable dosage form including, but not limited to, capsules, tablets, emulsions and aqueous suspensions, dispersions, and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

Alternatively or in addition, pharmaceutical compositions can be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

### Diagnostic Assays

The present invention provides, in part, methods, systems, and code for accurately classifying whether a biological sample contains an HSV virus and/or whether the levels of HSV infection are modulated (e.g., reduced), thereby indicative of the state of a disorder of interest, such as an active herpes outbreak. In some embodiments, the present invention is useful for classifying a sample (e.g., from a subject) as associated with or at risk for a disease, disorder, or condition associated with HSV infection using a statistical algorithm and/or empirical data (e.g., the presence, absence, or level of an HSV antigen).

An exemplary method for detecting the level of an HSV antigen or a fragment thereof, and thus useful for classifying whether a sample is associated with a disease or disorder mediated by HSV infection or a clinical subtype thereof, involves obtaining a biological sample from a test subject and contacting the biological sample with an antibody or antigen-binding fragment thereof of the present invention capable of binding to the HSV antigen such that the level of the HSV antigen can be detected in the biological sample. In some embodiments, at least one antibody or antigen-binding fragment thereof is used, wherein two, three, four, five, six, seven, eight, nine, ten, or more such antibodies or antibody fragments can be used in combination (e.g., in sandwich ELISAs) or in serial. In certain instances, the statistical algorithm is a single learning statistical classifier system. For example, a single learning statistical classifier system can be used to classify a sample as an HSV infected sample based upon a prediction or probability value and the presence or level of an HSV antigen. The use of a single learning statistical classifier system typically classifies the sample as an HSV infected sample with a sensitivity, specificity, positive predictive value, negative predictive value, and/or overall accuracy of at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

Other suitable statistical algorithms are well-known to those of skill in the art. For example, learning statistical classifier systems include a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used, preferably in tandem. Examples of learning statistical classifier systems include, but are not limited to, those using inductive learning (e.g., decision/classification trees such as random forests, classification and regression trees (C&RT), boosted trees, etc.), Probably Approximately Correct (PAC) learning, connectionist learning (e.g., neural networks (NN), artificial neural networks (ANN), neuro fuzzy networks (NFN), network structures, perceptrons such as multi-layer perceptrons, multi-layer feed-forward networks, applications of neural networks, Bayesian learning in belief networks, etc.), reinforcement learning (e.g., passive learning in a known environment such as naive learning, adaptive dynamic learning, and temporal difference learning, passive learning in an unknown environment, active learning in an unknown environment, learning action-value functions, applications of reinforcement learning, etc.), and genetic algorithms and evolutionary programming. Other learning statistical classifier systems include support vector machines (e.g., Kernel methods), multivariate adaptive regression splines (MARS), Levenberg-Marquardt algorithms, Gauss-Newton algorithms, mixtures of Gaussians, gradient descent algorithms, and learning vector quantization (LVQ). In certain embodiments, the method of the present invention further comprises sending the HSV infected sample classification results to a clinician, e.g., a histopathologist or an oncologist.

In another embodiment, the method of the present invention further provides a diagnosis in the form of a probability that the individual has a condition or disorder associated with HSV infection. For example, the individual can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having the condition or disorder. In yet another embodiment, the method of the present invention further provides a prognosis of the condition or disorder in the individual. In some instances, the method of classifying a sample as an HSV infected sample is further based on the symptoms (e.g., clinical factors) of the individual from which the sample is obtained. The symptoms or group of symptoms can be, for example, blisters around the mouth and lips or on the cornea of the eye (generally caused by an HSV-1 infection) or genital or rectal blisters (general indicative of an HSV-2 infection) and combinations thereof. Infections can also occur in the brain (herpes encephalitis) and the gastrointestinal tract, which can lead to severe illness. Additionally, some subjects that have atopic eczema can develop an HSV infection at the site of the eczema. In some embodiments, the diagnosis of an individual as having a condition or disorder associated with an HSV infection is followed by administering to the individual a therapeutically effective amount of a drug useful for treating one or more symptoms associated with the condition or disorder.

In one embodiment, the methods further involve obtaining a control biological sample (e.g., biological sample from a subject who does not have a condition or disorder associated with HSV infection), a biological sample from the subject during remission or before developing a condition or disorder associated with HSV infection, or a biological sample from the subject during treatment for developing a condition or disorder associated with HSV infection.

An exemplary method for detecting the presence or absence of an HSV antigen (e.g., gB or gD protein) or fragments thereof is an antibody of the present invention, or fragment thereof, capable of binding to an HSV antigen (e.g., HSV gB or gD protein), preferably an antibody with a detectable label. Antibodies can be polyclonal, or more preferably, monoclonal. Such agents can be labeled. The term “labeled”, with regard to the antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody. The term “biological sample” is intended to include tissues, cells, and biological fluids isolated from a subject, such as serum, as well as tissues, cells, and fluids present within a subject. That is, the detection method of the present invention can be used to detect an HSV antigen (e.g., HSV spike protein), in a biological sample in vitro as well as in vivo. In vitro techniques for detection of an HSV antigen (e.g., HSV gB or gD protein) include enzyme linked immunosorbent assays (ELISAs), Western blots, immunoprecipitations, immunohistochemistry (IHC), intracellular flow cytometry and related techniques, and immunofluorescence. Furthermore, in vivo techniques for detection of an HSV antigen (e.g., HSV gB or gD protein) or a fragment thereof include introducing into a subject a labeled antibody described herein. For example, the antibody can be labeled with a radioactive, luminescent, fluorescent, or other similar marker whose presence and location in a subject can be detected by standard imaging techniques, either alone or in combination with imaging for other molecules, such as markers of cell type (e.g., CD8+ T cell markers).

In one embodiment, the biological sample contains polypeptide molecules from the test subject. In certain embodiments, the sample may be serums, plasmas, cells, tissues, or body fluids isolated by conventional means from a subject.

In another embodiment, the methods further involve obtaining a control biological sample from a control subject, contacting the control sample with a compound or agent capable of detecting an HSV antigen, or a fragment thereof, such that the presence of the HSV antigen, or the fragment thereof, is detected in the biological sample, and comparing the presence of the HSV antigen, or the fragment thereof, in the control sample with the presence of the HSV antigen, or the fragment thereof in the test sample.

In still other embodiments, the antibodies can be associated with a component or device for the use of the antibodies in an ELISA or RIA. Non-limiting examples include antibodies immobilized on solid surfaces for use in these assays (e.g., linked and/or conjugated to a detectable label based on light or radiation emission as described above). In other embodiments, the antibodies are associated with a device or strip for detection an HSV antigen, or a fragment thereof by use of an immunochromatographic or immunochemical assay, such as in a “sandwich” or competitive assay, immunohistochemistry, immunofluorescence microscopy, and the like. Additional examples of such devices or strips are those designed for home testing or rapid point of care testing. Further examples include those that are designed for the simultaneous analysis of multiple analytes in a single sample. For example, an unlabeled antibody of the invention may be applied to a “capture” HSV antigen in a biological sample and the captured (or immobilized) HSV antigen may be bound to a labeled form of an antibody of the invention for detection. Other standard embodiments of immunoassays are well-known the skilled artisan, including assays based on, for example, immunodiffusion, immunoelectrophoresis, immunohistopathology, immunohistochemistry, and histopathology.

### Prognostic Assays

The diagnostic methods described herein can furthermore be utilized to identify subjects having or at risk of developing a disease, disorder, or condition associated with HSV infection.

The assays described herein, such as the preceding diagnostic assays or the following assays, can be utilized to identify a subject having or at risk of developing a disorder associated with HSV infection. Thus, the present invention provides a method for identifying a disorder associated with HSV infection in which a test sample is obtained from a subject and an HSV antigen (e.g., gB or gD protein) is detected, wherein the presence of an HSV antigen (e.g., gB or gD protein) is diagnostic for a subject having or at risk of developing the disorder associated with HSV infection. As used herein, a “test sample” refers to a biological sample obtained from a subject of interest. For example, a test sample can be a biological fluid (e.g., cerebrospinal fluid or serum), cell sample, or tissue, such as a nasal swab or lung tissue. In a preferred embodiment, the sample comprises cells from a lesion (e.g., a mouth or genital sore) caused by HSV.

Furthermore, the prognostic assays described herein can be used to determine whether a subject can be administered an agent (e.g., an agonist, antagonist, peptidomimetic, polypeptide, peptide, nucleic acid, small molecule, or other drug candidate) to treat such a disorder associated with an HSV infection. For example, such methods can be used to determine whether a subject can be effectively treated with one or a combination of agents. Thus, the present invention provides methods for determining whether a subject can be effectively treated with one or more agents for treating a disorder associated with an HSV infection in which a test sample is obtained and an HSV antigen (e.g., gB or gD protein) is detected (e.g., wherein the abundance of the HSV antigen is diagnostic for a subject that can be administered the agent to treat the disorder associated with an HSV infection).

The methods described herein may be performed, for example, by utilizing pre-packaged diagnostic kits comprising at least one antibody reagent described herein, which may be conveniently used, e.g., in clinical settings to diagnose patients exhibiting symptoms or family history of a disease or illness involving an HSV infection.

Furthermore, any cell type or tissue can be infected by HSV may be utilized in the prognostic assays described herein.

### Monitoring of Effects During Clinical Trials

Monitoring the influence of agents (e.g., compounds, drugs or small molecules) on the activity of an HSV antigen and/or HSV infection can be applied not only in basic drug screening, but also in clinical trials. For example, the effectiveness of an agent to reduce a level or activity of an HSV antigen, and/or HSV infection, can be monitored in clinical trials of subjects infected by HSV, detectable by the antibodies or fragments described herein. In such clinical trials, the level or activity of an HSV antigen, HSV infection, and/or symptoms or markers of the disorder of interest, can be used as a “read out” or marker of the phenotype of a particular cell, tissue, or system.

In one embodiment, the present invention provides a method for monitoring the effectiveness of treatment of a subject with an agent (e.g., an agonist, antagonist, peptidomimetic, polypeptide, peptide, nucleic acid, small molecule, or other drug candidate) including the steps of (i) obtaining a pre-administration sample from a subject prior to administration of the agent: (ii) detecting the level of an HSV antigen (e.g., HSV gB or gD protein), or a fragment thereof, in the preadministration sample: (iii) obtaining one or more post-administration samples from the subject: (iv) detecting the level of the HSV antigen (e.g., gB or gD protein), or the fragment thereof, in the post-administration samples: (v) comparing the level of the HSV antigen, or the fragment thereof, in the pre-administration sample with the HSV antigen in the post-administration sample or samples; and (vi) altering the administration of the agent to the subject accordingly. For example, increased administration of the agent may be desirable to decrease the level of the HSV antigen (e.g., gB or gD protein), i.e., to increase the effectiveness of the agent. According to such an embodiment, the HSV antigen (e.g., SARS-COV-2 spike protein) may be used as an indicator of the effectiveness of an agent, even in the absence of an observable phenotypic response. Similarly, the SARS-COV-2 antigen (e.g., SARS-COV-2 spike protein) analysis, such as by immunohistochemistry (IHC), can also be used to select patients who will receive treatment.

### Therapeutic Methods

In some instances, methods can include selection of a human subject who has or had a condition or disease and who exhibits or exhibited a positive immune response towards the condition or disease. In some instances, suitable subjects include, for example, subjects who have or had a condition or disease but that resolved the disease or an aspect thereof, present reduced symptoms of disease (e.g., relative to other subjects (e.g., the majority of subjects) with the same condition or disease), and/or that survive for extended periods of time with the condition or disease (e.g., relative to other subjects (e.g., the majority of subjects) with the same condition or disease), e.g., in an asymptomatic state (e.g., relative to other subjects (e.g., the majority of subjects) with the same condition or disease). In some instances, subjects can be selected if they have been vaccinated (e.g., previously vaccinated and/or vaccinated and re-vaccinated (e.g., received a booster vaccine)) against a condition or disease.

In some instances, subject selection can include obtaining a sample from a subject (e.g., a candidate subject) and testing the sample for an indication that the subject is suitable for selection. In some instances, the subject can be confirmed or identified, e.g. by a health care professional, as having had or having a condition or disease. In some instances, exhibition of a positive immune response towards a condition or disease can be made from patient records, family history, and/or detecting an indication of a positive immune response. In some instances multiple parties can be included in subject selection. For example, a first party can obtain a sample from a candidate subject and a second party can test the sample. In some instances, subjects can be selected and/or referred by a medical practitioner (e.g., a general practitioner). In some instances, subject selection can include obtaining a sample from a selected subject and storing the sample and/or using the in the methods disclosed herein. Samples can include, for example, cells or populations of cells.

Provided herein are methods for treating and/or preventing HSV, or diseases associated with HSV infection in a subject comprising administering to the subject a therapeutically effective amount of a composition comprising an antibody or antigen binding fragment thereof disclosed herein, or a pharmaceutical compositions described herein.

The disease can be any disease, disorder, or condition mediated by an HSV antigen and/or associated with HSV infection. In some embodiments, the subject has been diagnosed as having an HSV infection or as being predisposed to HSV infection.

In supporting the process of entry of the virus into the host cell, HSV glycoproteins interact with cell surface proteins. In particular, glycoprotein gB and gD are involved in viral attachment to a cell and the fusion of the viral envelope and the cell's plasma membrane. Accordingly, administering a monoclonal antibody described herein to a subject having a latent or active herpes infection can result in complete elimination of a latent and/or active infection. In some embodiments, administering a monoclonal antibody described herein to a subject having a latent infection can prevent the occurrence of an active infection. In some embodiments, administering the monoclonal antibodies to a subject suspected of being exposed or at risk of being exposed to HSV prevents a primary HSV infection. As used herein, a “primary HSV infection” refers to the first HSV infection a subject experiences from a particular transmission. In some embodiments, a subject may have more than one primary transmission if exposed to the virus by different hosts. In some embodiments, administering the monoclonal antibodies described herein to a subject having a primary HSV infection can prevent the establishment of a latent or active HSV infection.

The methods disclosed herein can be applied to a wide range of species, e.g., humans, non-human primates (e.g., monkeys), horses, cattle, pigs, sheep, deer, elk, goats, dogs, cats, mustelids, rabbits, guinea pigs, hamsters, rats, and mice.

By way of example, an anti-viral agent reduces viral load in a subject. In preferred embodiments, a therapeutically effective amount of the drug reduces viral load to the point of eliminating the virus. “Reducing viral load” means that administering an effective amount of the drug, alone or in combination with an anti-viral agent, results in a reduction in the levels of viral RNA and/or proteins, a reduction in the number of infected cells, a decrease in severity of at least one disease symptom, an increase in frequency and duration of disease symptom-free periods, a prevention of impairment or disability due to the disease affliction, or otherwise amelioration of disease symptoms in the patient. In addition, the terms “effective” and “effectiveness” with regard to a treatment includes both pharmacological effectiveness and physiological safety. Pharmacological effectiveness refers to the ability of the drug to reduce viral load in the patient. Physiological safety refers to the level of toxicity, or other adverse physiological effects at the cellular, organ and/or organism level (adverse effects) resulting from administration of the drug.

By way of example for the treatment of viral infection, a therapeutically effective amount or dosage of the drug preferably reduces viral load by at least about 20%, more preferably by at least about 40%, even more preferably by at least about 60%, and still more preferably by at least about 80% relative to untreated subjects. In the most preferred embodiments, a therapeutically effective amount or dosage of the drug completely inhibits viral infection, i.e., preferably reduces viral load by 100%. The ability of a compound to inhibit viral infection can be evaluated using the assays described infra. Alternatively, this property of a composition can be evaluated by examining the ability of the compound to inhibit viral infection, such inhibition can be measured in vitro by assays known to the skilled practitioner. In other preferred embodiments of the invention, viral infection may be observed and continue for a period of at least about 20 days, more preferably at least about 40 days, or even more preferably at least about 60 days.

In general, methods include selecting a subject at risk for or with a condition or disease. In some instances, the subject's condition or disease can be treated with a pharmaceutical composition disclosed herein. For example, in some instances, methods include selecting a subject with an HSV infection, e.g., wherein the subject's HSV infection can be treated by targeting an HSV antigen.

In some instances, treatments methods can include a single administration, multiple administrations, and repeating administration as required for the prophylaxis or treatment of the disease or condition from which the subject is suffering. In some instances treatment methods can include assessing a level of disease in the subject prior to treatment, during treatment, and/or after treatment. In some instances, treatment can continue until a decrease in the level of disease in the subject is detected.

The terms “administer,” “administering,” or “administration,” as used herein refers to implanting, absorbing, ingesting, injecting, or inhaling, the inventive antibodies, regardless of form. In some instances, one or more of the antibodies disclosed herein can be administered to a subject parenterally (e.g., by intravenous injection or infusion). For example, the methods herein include administration of an effective amount of compound or compound composition to achieve the desired or stated effect. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

For example, dosage regimens are adjusted to provide the optimum desired response (e.g., a therapeutic response). A single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. “Dosage unit form” as used herein refers to physically discrete units suited as unitary dosages for the subjects to be treated: each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of an antibody or antibody fragment described herein, the dosage ranges from about 0.0001 to 100 mg/kg, 0.001 to 50 mg/kg, 0.01 to 10 mg/kg, or 0.1 to 5 mg/kg, of the host body weight. For example dosages can be 0.01 mg/kg body weight, 0.03 mg/kg body weight, 0.05 mg/kg body weight, 0.08 mg/kg body weight, 0.1 mg/kg body weight, 0.3 mg/kg body weight, 0.5 mg/kg body weight, 0.8 mg/kg body weight, 1 mg/kg body weight, 3 mg/kg body weight, 5 mg/kg body weight, 8 mg/kg body weight, 10 mg/kg body weight, 20 mg/kg body weight, 30 mg/kg body weight, 40 mg/kg body weight, or 50 mg/kg body weight. An exemplary treatment regime entails administration once per week, once every two weeks, once every three weeks, once every four weeks, once a month, once every 3 months or once every three to 6 months. In some embodiments, dosage regimens for an antibody of the invention include 1 mg/kg body weight or 3 mg/kg body weight via intravenous administration, with the antibody being given using one of the following dosing schedules: (i) every four weeks for six dosages, then every three months: (ii) every three weeks: (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 g/ml.

Alternatively, pharmaceutical compositions can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Following administration, the subject can be evaluated to detect, assess, or determine their level of disease. In some instances, treatment can continue until a change (e.g., reduction) in the level of disease in the subject is detected.

Upon improvement of a patient's condition (e.g., a change (e.g., decrease) in the level of disease in the subject), a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms.

In some instances, the disclosure provides methods for detecting immune cells e.g., B cells and/or memory B cells, from a human subject. Such methods can be used, for example, to monitor the levels of immune cells e.g., B cells and/or memory B cells, in a human subject, e.g., following an event. Exemplary events can include, but are not limited to, detection of diseases, infection: administration of a therapeutic composition disclosed herein, administration of a therapeutic agent or treatment regimen, administration of a vaccine, induction of an immune response. Such methods can be used clinically and/or for research.

In some instances, the antibodies and/or antigen binding fragments disclosed herein can be administered in combination with compounds, drugs, and/or agents used for the treatment of an HSV infection. For example, the antibodies and/or antigen binding fragments disclosed herein may be administered in combination with anti-viral drugs, such as Zovirax® (acyclovir), Abreva® (1-docosanol), Valtrex® (valacyclovir), and Zovirax. In some instances, the antibodies and/or antigen binding fragments disclosed herein can be administered in combination with compounds, drugs, and/or agents used for the treatment of disease, disorder, or condition associated with an HSV infection. In some instances, therapeutic methods disclosed herein can include administration of one or more (e.g., one, two, three, four, five, or less than ten) compounds.

### EXAMPLES

The invention is further described in the following examples, which do not limit the scope of the invention described in the claims.

### Example 1: Donor Plasma has Neutralizing and ADCC-Inducing Activity Against HSV

In a previous study, a new HSV vaccine candidate (AgD) was shown to induce a high titer of antibodies capable of inducing antibody-dependent cellular cytotoxicity (ADCC) and not complement-independent antibodies in murine models. Serum from the vaccinated mice targeted multiple viral antigens including gB and demonstrated effector functions including induction of the complement system and antibody-dependent cellular cytotoxicity (ADCC). Importantly, the mice were protected from HSV-1 and HSV-2 infection (at LD90 and LD90×10), and latency was also inhibited.

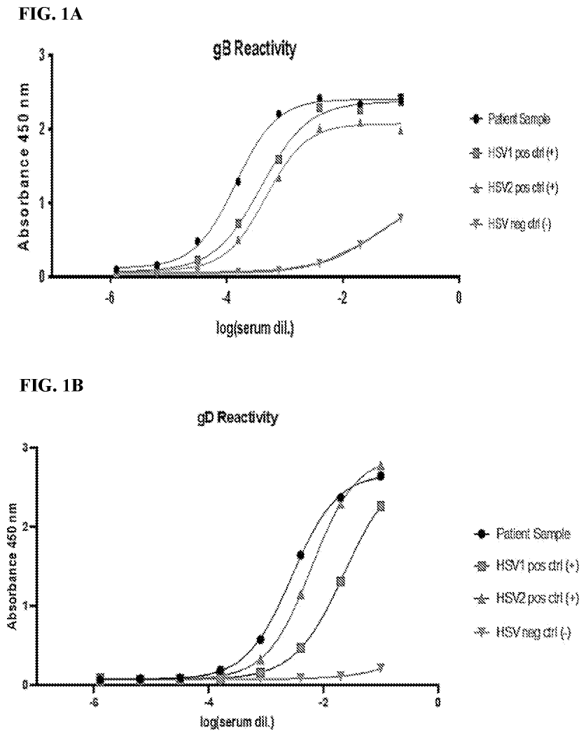

To determine if human subjects generate similar Fc-mediated responses to natural herpes simplex virus (HSV) infection, plasma was collected from HSV-positive subjects and assayed for reactivity to HSV antigens. The donor plasma demonstrated reactivity against gB and gD (FIGS. 1A and 1B) and had both neutralizing and ADCC responses (7.8 fold induction) in screening assays.

### Example 2: Isolated Anti-gD and gB Antibodies from Human Subject Sera have Neutralizing Activity

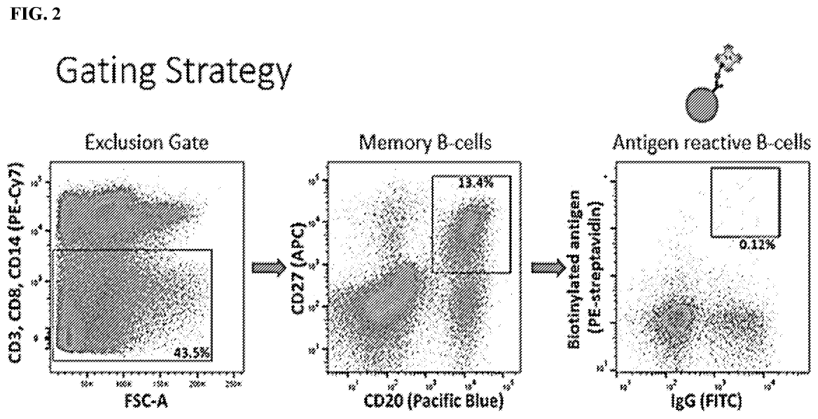

Fluorescence-activated cell sorting (FACS) was used to technology was used to isolate glycoprotein B (gB) and glycoprotein D (gD) reactive memory B-cells from peripheral blood mononuclear cells (PBMC) isolated from HSV-2 seropositive human subjects. The gating strategy for the FACS sorting is shown in FIG. 2.

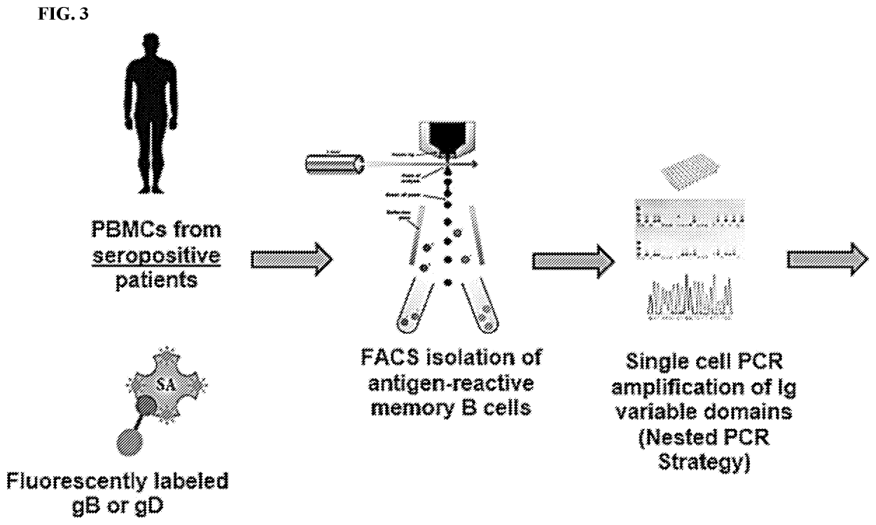

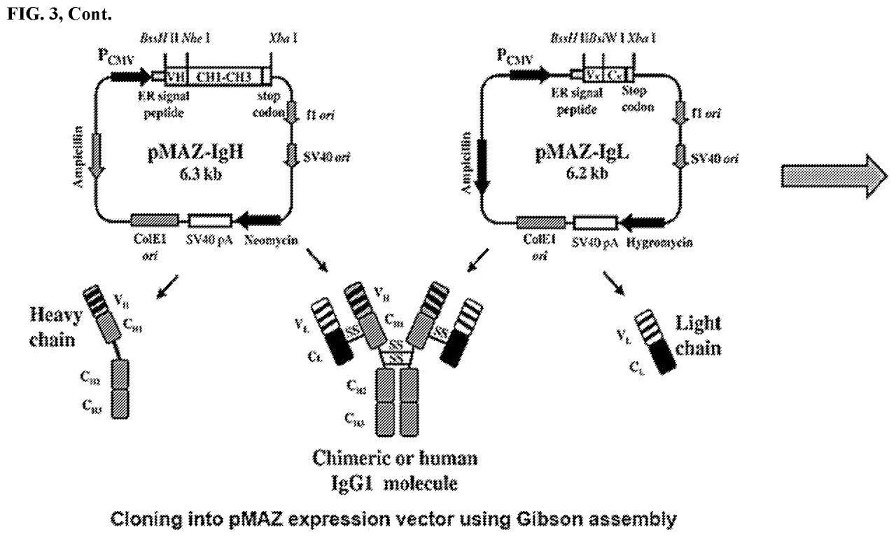

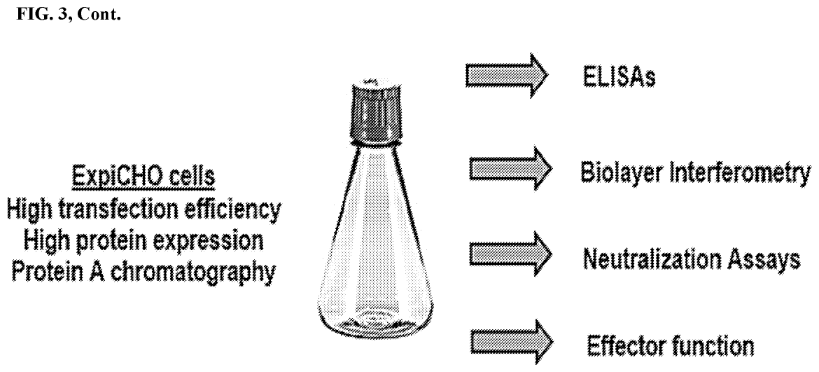

IgG variable domains were then PCR amplified from isolated single B cells, and the amplified sequences were cloned into pMAZ IgG1 expression vectors. Antibodies were then expressed in CHO cells and characterized (see FIG. 3 for a schematic of the antibody generation protocol).

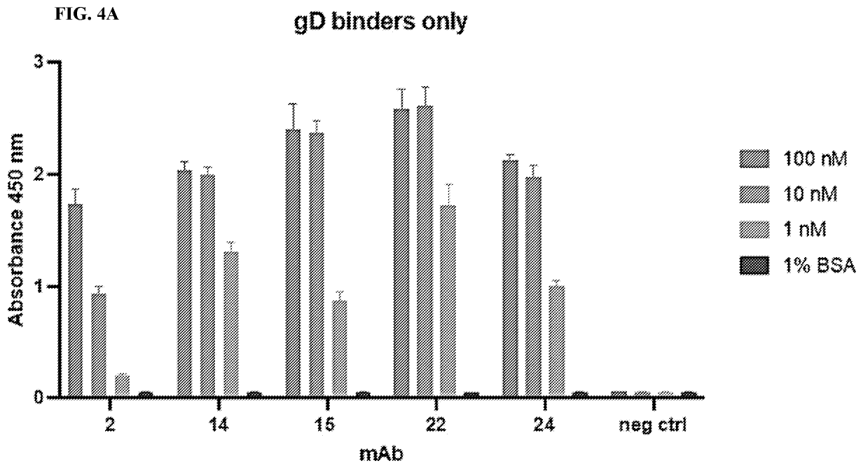

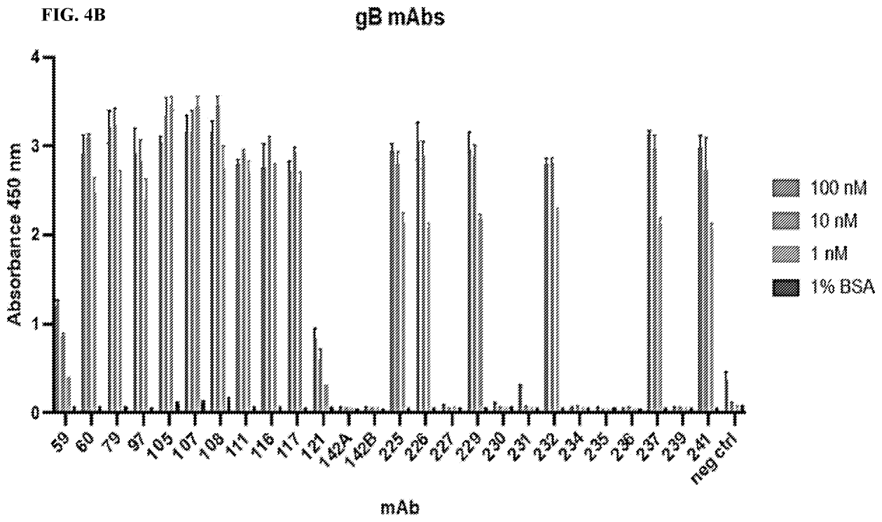

Several gB and gD binding human monoclonal antibodies (FIGS. 4A and 4B, respectively) were generated using the protocol shown in FIG. 3 (see Tables 2 and 3 in the specification for heavy and light chain sequences for the generated antibodies). The recombinant antibodies were characterized for neutralization activity, effector function, and to identify the epitopes that the antibodies bound.

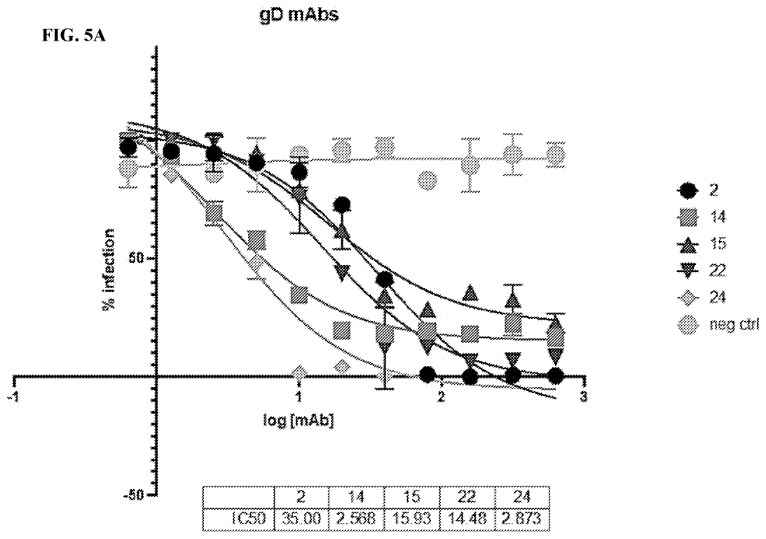

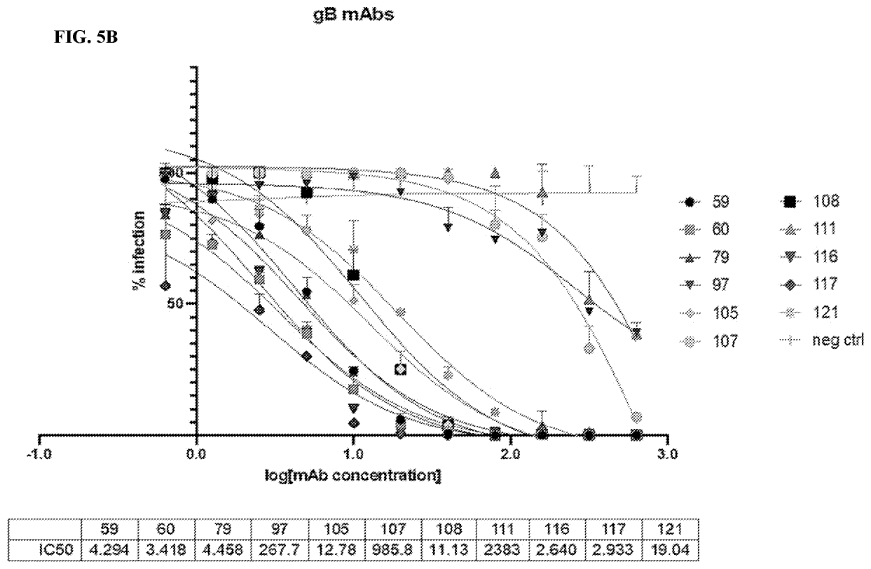

Referring to FIGS. 5A and 5B, several recombinant anti-gD and anti-gB antibodies, respectively, were identified that exhibited potent neutralizing activity. A concentration-dependent reduction in the percentage of infected cells was observed for each antibody assayed. The anti-gD antibodies assayed had similar neutralization profiles, but there was more variability in the neutralization profiles observed in the anti-gB antibodies assayed, with strong, moderate, and weak neutralizing antibodies identified.

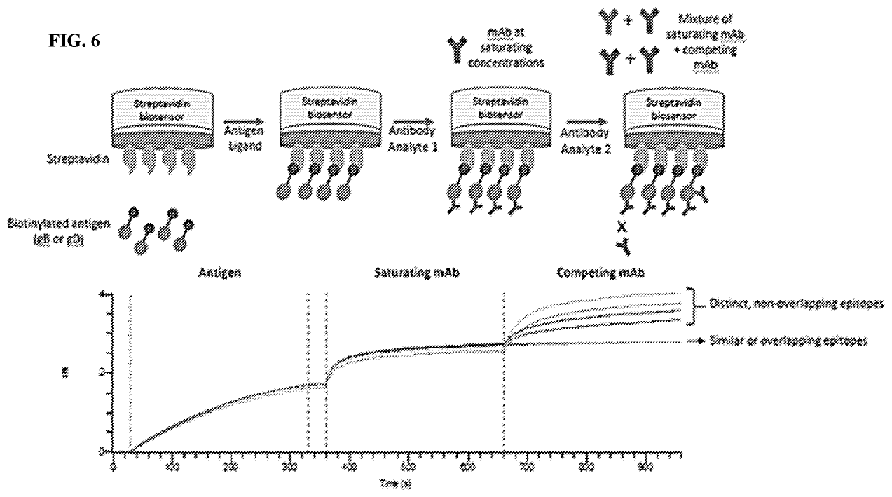

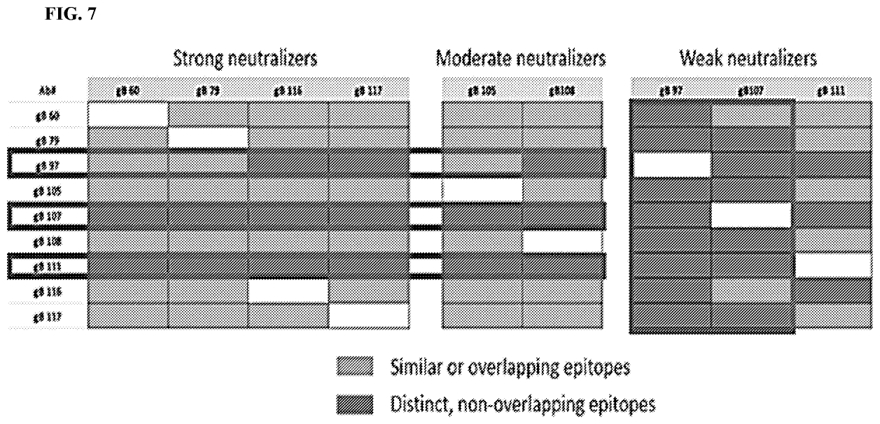

To identify epitopes recognized by the recombinant antibodies generated in this example, epitope binning was performed. Epitope binning clusters monoclonal antibodies based on the the specific epitope on the antigen that is recognized by the antibody. This technique is can be used to increase the likelihood of choosing lead antibodies with desired biological activities. In this example, epitope binning utilized biolayer interferometry (BLI), an optical technique that recognizes changes in the numbers of bound to a biosensor, which cause a shift in the interference pattern of white light reflected from two surfaces (FIG. 6). BLI analysis of strong, moderate, and weak neutralizing anti-gB antibodies showed that weakly neutralizing gB mAbs are more likely bind to different epitopes than strongly and moderately neutralizing mAbs and that strongly and moderately neutralizing gB mAbs compete (FIG. 7).

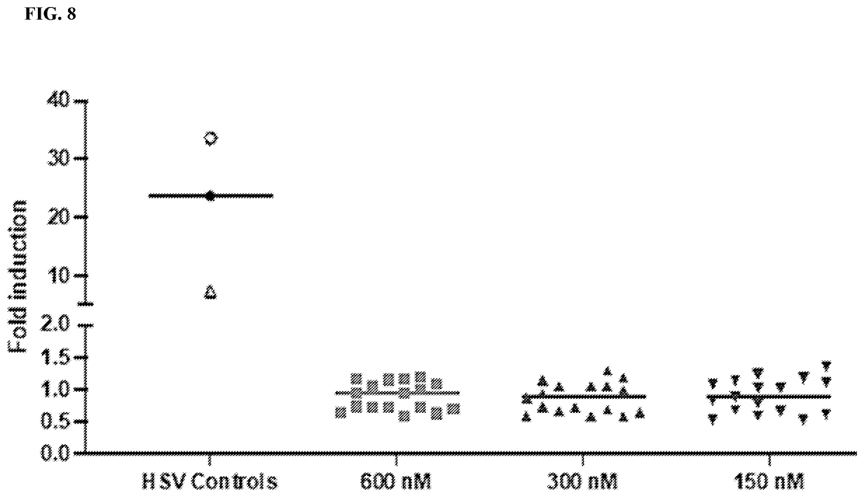

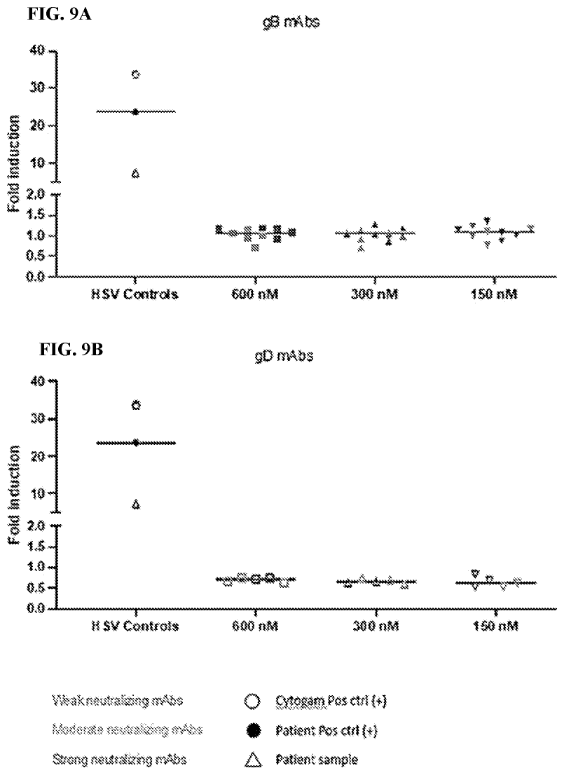

These monoclonal antibodies were also assayed to detect any antibody-mediated cellular cytotoxicity (ADCC) effector function. As shown in FIG. 8, the antibodies assayed appear to have minimal ADCC effector function. Referring to FIGS. 9A and 9B, the anti-gB and -gD antibodies did not activate the FcγRIIIa receptor, a biomarker of ADCC. This data suggests that anti-gB antibodies that induce ADCC are likely a subdominant population.

1. A recombinant monoclonal antibody that specifically binds to herpes simplex virus glycoprotein B (gB) comprising a heavy chain comprising an amino acid sequence having at least 95% identity to an amino acid sequence listed in Table 2 and a light chain comprising an amino acid sequence having at least 95% identity to an amino acid sequence listed in Table 2.

2. The recombinant monoclonal antibody of claim 1, wherein:
the heavy chain comprises an amino acid sequence has at least 95% identity to SEQ ID NO: 1 and the light chain comprises an amino acid sequence has at least 95% identity to SEQ ID NO: 54;
the heavy chain comprises an amino acid sequence has at least 95% identity to SEQ ID NO: 2 and the light chain comprises an amino acid sequence has at least 95% identity to SEQ ID NO: 55;
the heavy chain comprises an amino acid sequence has at least 95% identity to SEQ ID NO: 3 and the light chain comprises an amino acid sequence has at least 95% identity to SEQ ID NO: 56;
the heavy chain comprises an amino acid sequence has at least 95% identity to SEQ ID NO: 4 and the light chain comprises an amino acid sequence has at least 95% identity to SEQ ID NO: 57;
the heavy chain comprises an amino acid sequence has at least 95% identity to SEQ ID NO: 5 and the light chain comprises an amino acid sequence has at least 95% identity to SEQ ID NO: 58;
the heavy chain comprises an amino acid sequence has at least 95% identity to SEQ ID NO: 6 and the light chain comprises an amino acid sequence has at least 95% identity to SEQ ID NO: 59;
the heavy chain comprises an amino acid sequence has at least 95% identity to SEQ ID NO: 7 and the light chain comprises an amino acid sequence has at least 95% identity to SEQ ID NO: 60;
the heavy chain comprises an amino acid sequence has at least 95% identity to SEQ ID NO: 8 and the light chain comprises an amino acid sequence has at least 95% identity to SEQ ID NO: 61;
the heavy chain comprises an amino acid sequence has at least 95% identity to SEQ ID NO: 9 and the light chain comprises an amino acid sequence has at least 95% identity to SEQ ID NO: 62;
the heavy chain comprises an amino acid sequence has at least 95% identity to SEQ ID NO: 10 and the light chain comprises an amino acid sequence has at least 95% identity to SEQ ID NO: 63;
the heavy chain comprises an amino acid sequence has at least 95% identity to SEQ ID NO: 11 and the light chain comprises an amino acid sequence has at least 95% identity to SEQ ID NO: 64;
the heavy chain comprises an amino acid sequence has at least 95% identity to SEQ ID NO: 16 and the light chain comprises an amino acid sequence has at least 95% identity to SEQ ID NO: 65;
the heavy chain comprises an amino acid sequence has at least 95% identity to SEQ ID NO: 17 and the light chain comprises an amino acid sequence has at least 95% identity to SEQ ID NO: 66;
the heavy chain comprises an amino acid sequence has at least 95% identity to SEQ ID NO: 18 and the light chain comprises an amino acid sequence has at least 95% identity to SEQ ID NO: 67;
the heavy chain comprises an amino acid sequence has at least 95% identity to SEQ ID NO: 19 and the light chain comprises an amino acid sequence has at least 95% identity to SEQ ID NO: 68;
the heavy chain comprises an amino acid sequence has at least 95% identity to SEQ ID NO: 20 and the light chain comprises an amino acid sequence has at least 95% identity to SEQ ID NO: 69;
the heavy chain comprises an amino acid sequence has at least 95% identity to SEQ ID NO: 21 and the light chain comprises an amino acid sequence has at least 95% identity to SEQ ID NO: 70;
the heavy chain comprises an amino acid sequence has at least 95% identity to SEQ ID NO: 22 and the light chain comprises an amino acid sequence has at least 95% identity to SEQ ID NO: 71;
the heavy chain comprises an amino acid sequence has at least 95% identity to SEQ ID NO: 23 and the light chain comprises an amino acid sequence has at least 95% identity to SEQ ID NO: 72;
the heavy chain comprises an amino acid sequence has at least 95% identity to SEQ ID NO: 24 and the light chain comprises an amino acid sequence has at least 95% identity to SEQ ID NO: 73;
the heavy chain comprises an amino acid sequence has at least 95% identity to SEQ ID NO: 25 and the light chain comprises an amino acid sequence has at least 95% identity to SEQ ID NO: 74;
the heavy chain comprises an amino acid sequence has at least 95% identity to SEQ ID NO: 26 and the light chain comprises an amino acid sequence has at least 95% identity to SEQ ID NO: 75;
the heavy chain comprises an amino acid sequence has at least 95% identity to SEQ ID NO: 27 and the light chain comprises an amino acid sequence has at least 95% identity to SEQ ID NO: 76;
the heavy chain comprises an amino acid sequence has at least 95% identity to SEQ ID NO: 28 and the light chain comprises an amino acid sequence has at least 95% identity to SEQ ID NO: 77;
the heavy chain comprises an amino acid sequence has at least 95% identity to SEQ ID NO: 29 and the light chain comprises an amino acid sequence has at least 95% identity to SEQ ID NO: 80;
the heavy chain comprises an amino acid sequence has at least 95% identity to SEQ ID NO: 30 and the light chain comprises an amino acid sequence has at least 95% identity to SEQ ID NO: 81;
the heavy chain comprises an amino acid sequence has at least 95% identity to SEQ ID NO: 31 and the light chain comprises an amino acid sequence has at least 95% identity to SEQ ID NO: 82;
the heavy chain comprises an amino acid sequence has at least 95% identity to SEQ ID NO: 32 and the light chain comprises an amino acid sequence has at least 95% identity to SEQ ID NO: 84;
the heavy chain comprises an amino acid sequence has at least 95% identity to SEQ ID NO: 33 and the light chain comprises an amino acid sequence has at least 95% identity to SEQ ID NO: 85;
the heavy chain comprises an amino acid sequence has at least 95% identity to SEQ ID NO: 34 and the light chain comprises an amino acid sequence has at least 95% identity to SEQ ID NO: 86;
the heavy chain comprises an amino acid sequence has at least 95% identity to SEQ ID NO: 35 and the light chain comprises an amino acid sequence has at least 95% identity to SEQ ID NO: 87;
the heavy chain comprises an amino acid sequence has at least 95% identity to SEQ ID NO: 36 and the light chain comprises an amino acid sequence has at least 95% identity to SEQ ID NO: 88;
the heavy chain comprises an amino acid sequence has at least 95% identity to SEQ ID NO: 37 and the light chain comprises an amino acid sequence has at least 95% identity to SEQ ID NO: 90;
the heavy chain comprises an amino acid sequence has at least 95% identity to SEQ ID NO: 38 and the light chain comprises an amino acid sequence has at least 95% identity to SEQ ID NO: 91;
the heavy chain comprises an amino acid sequence has at least 95% identity to SEQ ID NO: 39 and the light chain comprises an amino acid sequence has at least 95% identity to SEQ ID NO: 92;
the heavy chain comprises an amino acid sequence has at least 95% identity to SEQ ID NO: 40 and the light chain comprises an amino acid sequence has at least 95% identity to SEQ ID NO: 93;
the heavy chain comprises an amino acid sequence has at least 95% identity to SEQ ID NO: 41 and the light chain comprises an amino acid sequence has at least 95% identity to SEQ ID NO: 94;
the heavy chain comprises an amino acid sequence has at least 95% identity to SEQ ID NO: 42 and the light chain comprises an amino acid sequence has at least 95% identity to SEQ ID NO: 95;
the heavy chain comprises an amino acid sequence has at least 95% identity to SEQ ID NO: 43 and the light chain comprises an amino acid sequence has at least 95% identity to SEQ ID NO: 96;
the heavy chain comprises an amino acid sequence has at least 95% identity to SEQ ID NO: 45 and the light chain comprises an amino acid sequence has at least 95% identity to SEQ ID NO: 97;
the heavy chain comprises an amino acid sequence has at least 95% identity to SEQ ID NO: 46 and the light chain comprises an amino acid sequence has at least 95% identity to SEQ ID NO: 98;
the heavy chain comprises an amino acid sequence has at least 95% identity to SEQ ID NO: 47 and the light chain comprises an amino acid sequence has at least 95% identity to SEQ ID NO: 99;
the heavy chain comprises an amino acid sequence has at least 95% identity to SEQ ID NO: 48 and the light chain comprises an amino acid sequence has at least 95% identity to SEQ ID NO: 100;
the heavy chain comprises an amino acid sequence has at least 95% identity to SEQ ID NO: 49 and the light chain comprises an amino acid sequence has at least 95% identity to SEQ ID NO: 101;
the heavy chain comprises an amino acid sequence has at least 95% identity to SEQ ID NO: 50 and the light chain comprises an amino acid sequence has at least 95% identity to SEQ ID NO: 102;
the heavy chain comprises an amino acid sequence has at least 95% identity to SEQ ID NO: 51 and the light chain comprises an amino acid sequence has at least 95% identity to SEQ ID NO: 103;
the heavy chain comprises an amino acid sequence has at least 95% identity to SEQ ID NO: 52 and the light chain comprises an amino acid sequence has at least 95% identity to SEQ ID NO: 104;
the heavy chain comprises an amino acid sequence has at least 95% identity to SEQ ID NO: 53 and the light chain comprises an amino acid sequence has at least 95% identity to SEQ ID NO: 105; or
the heavy chain comprises an amino acid sequence has at least 95% identity to SEQ ID NO: 153 and the light chain comprises an amino acid sequence has at least 95% identity to SEQ ID NO: 106.

3-50. (canceled)

51. The recombinant monoclonal antibody of claim 1 wherein the heavy chain comprises an amino acid sequence listed in Table 2.

52. The recombinant monoclonal antibody of claim 51, wherein the heavy chain comprises an amino acid sequence of SEQ ID NO: 12-15 or 44.

53. The recombinant monoclonal antibody of claim 1 wherein the light chain comprises an amino acid sequence listed in Table 2.

54. The recombinant monoclonal antibody of claim 53, wherein the light chain comprises an amino acid sequence of SEQ ID NO: 78, 79, 83, or 89.

55. A recombinant monoclonal antibody that specifically binds to herpes simplex virus glycoprotein D (gD) comprising a heavy chain comprising an amino acid sequence having at least 95% identity to an amino acid sequence listed in Table 3 and a light chain comprising an amino acid sequence having at least 95% identity to an amino acid sequence listed in Table 3.

56. The recombinant monoclonal antibody of claim 55, wherein:
the heavy chain comprises an amino acid sequence has at least 95% identity to SEQ ID NO: 107 and the light chain comprises an amino acid sequence has at least 95% identity to SEQ ID NO: 112;
the heavy chain comprises an amino acid sequence has at least 95% identity to SEQ ID NO: 108 and the light chain comprises an amino acid sequence has at least 95% identity to SEQ ID NO: 113;
the heavy chain comprises an amino acid sequence has at least 95% identity to SEQ ID NO: 109 and the light chain comprises an amino acid sequence has at least 95% identity to SEQ ID NO: 114;
the heavy chain comprises an amino acid sequence has at least 95% identity to SEQ ID NO: 110 and the light chain comprises an amino acid sequence has at least 95% identity to SEQ ID NO: 115; or
the heavy chain comprises an amino acid sequence has at least 95% identity to SEQ ID NO: 111 and the light chain comprises an amino acid sequence has at least 95% identity to SEQ ID NO: 116.

57-60. (canceled)

61. A pharmaceutical composition comprising the recombinant monoclonal antibody of claim 1 and a pharmaceutically acceptable carrier.

62. A method of treating a subject having or suspected of having a herpes simplex virus (HSV) infection, the method comprising administering to the subject the monoclonal antibody of claim 1.

63. A method of preventing a herpes simplex virus (HSV) infection in a subject exposed to or at risk of being exposed to HSV, the method comprising administering to the subject the monoclonal antibody of claim 1.

64. A method of treating a herpes simplex virus (HSV) outbreak in a subject, the method comprising administering to the subject the monoclonal antibody of claim 1.

65. A method of preventing a herpes simplex virus (HSV) outbreak in a subject having or suspected of having a latent HSV infection, the method comprising administering to the subject the monoclonal antibody of claim 1.

66. The method of claim 62, wherein the HSV infection is a latent infection.

67. (canceled)

68. (canceled)

69. The method of claim 62, wherein the monoclonal antibody is administered intravenously, intraperitoneally, intramuscularly, subcutaneously, intranasally, orally, vaginally, rectally, sublingually, or topically.

70. (canceled)

71. A method of eliciting a cellular immune response and antibody-dependent cellular cytotoxicity against a herpes simplex virus (HSV) infected cell, the method comprising contacting the cell with the monoclonal antibody of claim 1.

72. (canceled)

73. (canceled)

74. (canceled)

75. (canceled)

76. The monoclonal antibody of claim 1 further comprising a detectable label.

77. A nucleic acid molecule encoding the monoclonal antibody of claim 1.

78. (canceled)

79. A host cell comprising the nucleic acid molecule of claim 77.

80. A method of producing the monoclonal antibody of claim 1, wherein the method comprises the steps of: (i) culturing a host cell comprising a nucleic acid comprising a sequence encoding the monoclonal antibody of claim 1 under conditions suitable to allow expression of said monoclonal antibody; and (ii) recovering the expressed monoclonal antibody.
